(19) United States
(12) Patent Application Publication
(10) Pub. No.: US 2024/0337937 A1
(43) Pub. Date: Oct. 10, 2024

(54) RESIST COMPOSITION AND METHOD FOR FORMING RESIST PATTERN

(71) Applicant: TOKYO OHKA KOGYO CO., LTD., Kawasaki-shi (JP)

(72) Inventors: Hiroki KATO, Kawasaki-shi (JP); Tetsuo FUJINAMI, Kawasaki-shi (JP); Shuichi ISHII, Kawasaki-shi (JP)

(21) Appl. No.: 18/575,649

(22) PCT Filed: Aug. 1, 2022
(86) PCT No.: PCT/JP2022/029513

§ 371 Date: Dec. 29, 2023

(30) Foreign Application Priority Data
Aug. 4, 2021 (JP) 2021-128286

(51) Int. Cl. G03F 7/039; C08F 212/14; C08F 220/18; G03F 7/004; G03F 7/038

(52) U.S. Cl. CPC G03F 7/039; C08F 212/22; C08F 212/24; C08F 220/1806; G03F 7/0045; G03F 7/038

(57) ABSTRACT

A resist composition including a resin component having a constitutional unit represented by General Formula (a0-1) and a compound represented by General Formula (d0-1). In General Formula (a0-1), R⁰ represents a hydrogen atom, an alkyl group, a halogen atom, or a halogenated alkyl group; Va^{x0} represents a single bond or a divalent linking group; Wa represents a divalent aromatic hydrocarbon group; Va⁰ represents a divalent hydrocarbon group; nₐ₀ represents an integer of 0 to 2; and Ra⁰⁰ represents an acid dissociable group. In General Formula (d0-1), X⁰ represents a bromine atom or an iodine atom; R^{m} represents a hydroxy group, an alkyl group, a fluorine atom, or a chlorine atom; nd1 represents an integer of 1 to 5; nd2 represents an integer of 0 to 4; Yd⁰ represents a divalent linking group or a single bond; M^{m+} represents an m-valent organic cation; and m represents an integer of 1 or greater

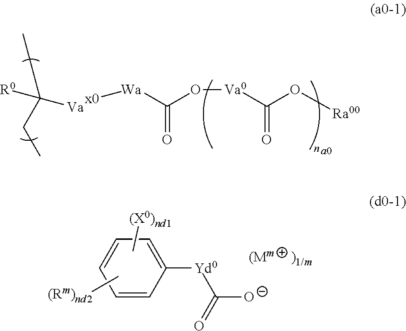

### TECHNICAL FIELD

The present invention relates to a resist composition and a resist pattern formation method.

Priority is claimed on Japanese Patent Application No. 2021-128286, filed on Aug. 4, 2021, the content of which is incorporated herein by reference.

### BACKGROUND ART

In recent years, in the manufacture of semiconductor elements and liquid crystal display elements, advances in lithography technologies have led to rapid progress in the field of pattern miniaturization. These pattern miniaturization techniques typically involve shortening the wavelength (increasing the energy) of the exposure light source.

Resist materials require lithography characteristics such as a high resolution that enables reproduction of patterns with minute dimensions, and a high level of sensitivity to these kinds of exposure light sources.

As a resist material that satisfies these requirements, a chemically amplified resist composition containing a base material component whose solubility in a developing solution is changed due to an action of an acid and an acid generator component that generates an acid upon light exposure has been used.

In the chemically amplified resist composition, a resin having a plurality of constitutional units is typically used in order to improve lithography characteristics and the like.

Further, various kinds of acid generators have been suggested as the acid generator used in a chemically amplified resist composition. Examples of such known acid generators include an onium salt-based acid generator such as an iodonium salt or a sulfonium salt, an oxime sulfonate-based acid generator, a diazomethane-based acid generator, a nitrobenzyl sulfonate-based acid generator, an iminosulfonate-based acid generator, and a disulfone-based acid generator.

In the resist pattern formation, the behavior of an acid generated from an acid generator component upon light exposure is considered as one factor that has a great influence on lithography characteristics.

Meanwhile, in the related art, a resist composition obtained by employing a resin having a specific structure or an acid generator having a specific structure in order to control diffusion of an acid generated upon light exposure has been suggested (for example, see Patent Document 1).

Alternatively, in the related art, a chemically amplified resist composition obtained by combining an acid generator component with an acid diffusion control agent that controls diffusion of an acid generated from the acid generator component upon light exposure has been suggested.

### CITATION LIST

### Patent Document

### [Patent Document I]

Japanese Unexamined Patent Application, First Publication No. 2019-219470

### SUMMARY OF INVENTION

### Technical Problem

In the future, with further miniaturization of a pattern, a resist film will be thinned, and a resist material is required to have further improved etching resistance in a case where etching is performed by using a resist pattern as a mask. In addition, the resist material is required to have high sensitivity to radiation as miniaturization of the pattern proceeds. Further, as the pattern dimensions decrease, unexposed portions of the resist film are dissolved due to development (reduction of a developed film), and the amount of the pattern remaining film is likely to decrease. With respect to these required characteristics, resist compositions of the related art are required to have further improved performance.

The present invention has been made in consideration of the above-described circumstances, and an object thereof is to provide a resist composition capable of further improving etching resistance, increasing sensitivity, and suppressing film reduction of a pattern, and a resist pattern formation method using the resist composition.

### Solution to Problem

In order to achieve the above-described object, the present invention employs the following configurations.

That is, according to a first aspect of the present invention, there is provided a resist composition which generates an acid upon light exposure and whose solubility in a developing solution is changed due to an action of an acid, the resist composition including: a resin component (A1) whose solubility in a developing solution is changed due to an action of an acid; and a compound (D0) represented by General Formula (d0-1), in which the resin component (A1) has a constitutional unit (a0) represented by General Formula (a0-1).

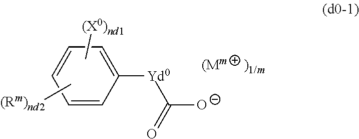

[In the formula, X⁰ represents a bromine atom or an iodine atom. R^{m} represents a hydroxy group, an alkyl group, a fluorine atom, or a chlorine atom. nd1 represents an integer of 1 to 5, and nd2 represents an integer of 0 to 4, where 1≤nd1+nd2≥5 is satisfied. Yd⁰ represents a divalent linking group or a single bond. M^{m+} represents an m-valent organic cation. m represents an integer of 1 or greater.]

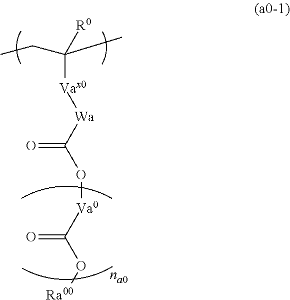

[In the formula, R^{o} represents a hydrogen atom, an alkyl group having 1 to 5 carbon atoms, a halogen atom, or a halogenated alkyl group having 1 to 5 carbon atoms. Va^{x0} represents a single bond or a divalent linking group. Wa represents a divalent aromatic hydrocarbon group which may have a substituent. Va⁰ represents a divalent hydrocarbon group which may have an ether bond. nₐ₀ represents an integer of 0 to 2. Ra⁰⁰ represents an acid dissociable group.]

According to a second aspect of the present invention, there is provided a method of forming a resist pattern, including: a step of forming a resist film on a support using the resist composition according to the first aspect; a step of exposing the resist film to light; and a step of developing the resist film exposed to light to form a resist pattern.

### Advantageous Effects of Invention

According to the present invention, it is possible to provide a resist composition capable of further improving etching resistance, increasing sensitivity, and suppressing film reduction of a pattern, and a resist pattern formation method using the resist composition.

### DESCRIPTION OF EMBODIMENTS

In the present specification and the present claims, the term “aliphatic” is a relative concept used in relation to the term “aromatic”, and defines a group or compound that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom. The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “base material component” denotes an organic compound having a film-forming ability. Organic compounds used as the base material component are classified into non-polymers and polymers. As the non-polymers, those having a molecular weight of 500 or greater and less than 4000 are typically used. Hereinafter, the term “low-molecular-weight compound” denotes a non-polymer having a molecular weight of 500 or greater and less than 4000. As the polymer, those having a molecular weight of 1000 or greater are typically used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, the acrylic acid ester includes itaconic acid diester in which the substituent (R^{αx}) has been substituted with a substituent having an ester bond and α-hydroxyacryl ester in which the substituent (R^{αx}) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified. Examples of the substituent that substitutes the hydrogen atom at the α-position of hydroxystyrene include those for R^{αx}.

The term “acid decomposable group” indicates a group having acid decomposability in which at least a part of a bond in the structure of the acid decomposable group can be cleaved due to the action of an acid.

Examples of the acid decomposable group whose polarity is increased due to the action of an acid include groups which are decomposed due to the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

In the present specification and the present claims, asynnetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed by the action of the acid.

The resist composition according to the present embodiment contains a base material component (A) whose solubility in a developing solution is changed due to the action of an acid (hereinafter, also referred to as “component (A)”) and a compound (D0) represented by General Formula (d0-1) (hereinafter, also referred to as “component (D0)”).

In a case where a resist film is formed using the resist composition according to the present embodiment and the formed resist film is subjected to selective exposure, an acid is generated at exposed portions of the resist film, and the generated acid acts on the component (A) to change the solubility of the component (A) in a developing solution. On the other hand, the solubility of the component (A) in a developing solution is not changed at unexposed portions of the resist film, which generates the difference in solubility in the developing solution between exposed portions and unexposed portions of the resist film. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone. In the present specification, a resist composition which forms a positive-tone resist pattern by dissolving and removing the exposed portion of the resist film is referred to as a positive-tone resist composition, and a resist composition which forms a negative-tone resist pattern by dissolving and removing the unexposed portion of the resist film is referred to as a negative-tone resist composition.

The resist composition of the present embodiment may be a positive-tone resist composition or a negative-tone resist composition.

Further, in the formation of a resist pattern, the resist composition according to the present embodiment can be applied to an alkali developing process using an alkali developing solution in the developing treatment, or a solvent developing process using an organic developing solution in the developing treatment.

The resist composition of the present embodiment has a function of generating an acid upon light exposure, the component (A) may generate an acid upon light exposure, and an additive component that is separately blended from the component (A) may generate an acid upon light exposure.

Specifically, (1) the resist composition of the present embodiment may further contain an acid generator component (B) that generates an acid upon light exposure (hereinafter, referred to as “component (B)”), (2) the component (A) may be a component that generates an acid upon light exposure, and (3) the component (A) is a component that generates an acid upon light exposure and may further contain the component (B). That is, in cases of (2) and (3), the component (A) is “base material component that generates an acid upon light exposure and whose solubility in a developing solution is changed due to the action of an acid”. In a case where the component (A) is a base material component which generates an acid upon light exposure and whose solubility in a developing solution is changed due to the action of the acid, it is preferable that a component (A1) described below is a polymer compound which generates an acid upon light exposure and whose solubility in a developing solution is changed due to the action of the acid. As such a polymer compound, a resin having a constitutional unit that generates an acid upon light exposure can be used. As the constitutional unit that generates an acid upon light exposure, those which have been known can be used.

Among the above-described conditions, the resist composition according to the present embodiment is preferably under the condition (1), that is, it is preferable that the resist composition contains the component (A), the component (D0), and the component (B).

### <Component (A)>

In the resist composition according to the present embodiment, the component (A) contains a resin component (A1) (hereinafter, also referred to as “component (A1)”) whose solubility in a developing solution is changed due to the action of an acid. Since the polarity of the base material component before and after the light exposure is changed by using the component (A1), an excellent development contrast can be obtained not only in an alkali developing process but also in a solvent developing process.

As the component (A), another polymer compound and/or a low-molecular-weight compound may be used in combination with the component (A1).

In a case of applying an alkali developing process, the base material component having the component (A1) is insoluble in an alkali developing solution before light exposure, but in a case where an acid is generated from the component (B) upon light exposure, the action of this acid causes an increase in the polarity of the base material component, thereby increasing the solubility of the component (A1) in an alkali developing solution. Therefore, in a case where selective light exposure is performed on a resist film formed by coating a support with the resist composition in the resist pattern formation, the exposed portion of the resist film is changed from being insoluble to being soluble in an alkali developing solution, whereas the unexposed portion of the resist film remains insoluble in an alkali developing solution, and thus a positive-tone resist pattern is formed by performing alkali development.

Meanwhile, in a case of a solvent developing process, the base material component having the component (A1) exhibits high solubility in an organic developing solution before light exposure. For example, in a case where an acid is generated from the component (B) upon light exposure, the polarity of the component (A1) is increased by the action of the generated acid, thereby decreasing the solubility of the component (A1) in an organic developing solution. Therefore, in a case where selective light exposure is performed on a resist film formed by coating a support with the resist composition in the resist pattern formation, the exposed portion of the resist film is changed from being soluble to being insoluble in an organic developing solution, whereas the unexposed portion of the resist film remains soluble in an organic developing solution. Therefore, a negative-tone resist pattern is formed by performing development using an organic developing solution so that a contrast is imparted between the exposed portion and the unexposed portion.

In the resist composition according to the present embodiment, the component (A) may be used alone or in combination of two or more kinds thereof.

### In Regard to Component (A1)

The component (A1) in the resist composition according to the present embodiment is a resin component whose solubility in a developing solution is changed due to the action of an acid and has a constitutional unit (a0) represented by General formula (a0-1).

Constitutional Unit (a0)>>

The constitutional unit (a0) contains an acid decomposable group whose polarity is increased due to the action of an acid.

The term “acid decomposable group” denotes a group having acid decomposability in which at least a part of a bond in the structure of the acid decomposable group can be cleaved due to the action of an acid. In the constitutional unit (a0), a bond between an acid dissociable group Ra⁰⁰ and an oxygen atom (—O—) of a carbonyloxy group (—C(═O)—O—) adjacent to the acid dissociable group Ra⁰⁰ is cleaved due to the action of an acid, and the polarity is increased due to generation of a polar group (carboxy group) having high polarity.

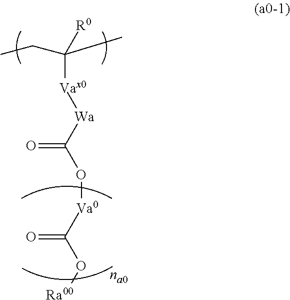

[In the formula, R⁰ represents a hydrogen atom, an alkyl group having 1 to 5 carbon atoms, a halogen atom, or a halogenated alkyl group having 1 to 5 carbon atoms. Va^{x0} represents a single bond or a divalent linking group. Wa represents a divalent aromatic hydrocarbon group which may have a substituent. Va⁰ represents a divalent hydrocarbon group which may have an ether bond. nₐ₀ represents an integer of 0 to 2. Ra⁰⁰ represents an acid dissociable group.]

In General Formula (a0-1), R⁰ represents a hydrogen atom, an alkyl group having 1 to 5 carbon atoms, a halogen atom, or a halogenated alkyl group having 1 to 5 carbon atoms.

As the alkyl group having 1 to 5 carbon atoms as R⁰, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group.

Examples of the halogen atom as R⁰ include a fluorine atom, a chlorine atom, a bromine atom, and an iodine atom. Among these, a fluorine atom is preferable.

The halogenated alkyl group having 1 to 5 carbon atoms as R⁰ is a group in which some or all hydrogen atoms of the above-described alkyl group having 1 to 5 carbon atoms have been substituted with halogen atoms.

R⁰ represents preferably a hydrogen atom, an alkyl group having 1 to 5 carbon atoms, a fluorine atom, or a fluorinated alkyl group having 1 to 5 carbon atoms, and from the viewpoint of industrial availability, more preferably a hydrogen atom, a methyl group, or a fluorine atom, and particularly preferably a hydrogen atom.

In Formula (a0-1), Va^{x0} represents a single bond or a divalent linking group.

The divalent linking group as Va^{x0} is not particularly limited, and suitable examples thereof include a divalent hydrocarbon group which may have a substituent and a divalent linking group having a hetero atom.

Divalent Hydrocarbon Group which May have Substituent:

In a case where Va^{x0} represents a divalent hydrocarbon group which may have a substituent, the hydrocarbon group may be an aliphatic hydrocarbon group or an aromatic hydrocarbon group.

### Aliphatic Hydrocarbon Group as Va^{x0}

The aliphatic hydrocarbon group indicates a hydrocarbon group that has no aromaticity. The aliphatic hydrocarbon group may be saturated or unsaturated. In general, it is preferable that the aliphatic hydrocarbon group is saturated. Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a hetero atom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as those described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, and a methoxy group, an ethoxy group, an n-propoxy group, an iso-propoxy group, an n-butoxy group, or a tert-butoxy group is more preferable, and a methoxy group or an ethoxy group is still more preferable.

Examples of the halogen atom as the substituent include a fluorine atom, a chlorine atom, a bromine atom, and an iodine atom. Among these, a fluorine atom is preferable.

Examples of the halogenated alkyl group as the substituent include groups in which some or all hydrogen atoms in the above-described alkyl groups have been substituted with the above-described halogen atoms.

In the cyclic aliphatic hydrocarbon group, some of the carbon atoms constituting the ring structure thereof may be substituted with a substituent having a hetero atom. As the substituent having a hetero atom, —O—, —C(═O)—O—, —S—, —S(═O)₂—, or —S(═O)₂—O— is preferable.

### Aromatic Hydrocarbon Group as Va^{x0}

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 10 carbon atoms. Here, the number of carbon atoms in a substituent is not included in the number of carbon atoms.

Specific examples of the aromatic ring include aromatic hydrocarbon rings such as benzene, naphthalene, anthracene, and phenanthrene; and aromatic heterocyclic rings in which some of the carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (such as biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in arylalkyl groups such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of the substituent include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Hetero Atom:

In a case where Va^{x0} represents a divalent linking group having a hetero atom, preferred examples of the linking group include —O—, —C(═O)—O—, —O—C(═O)—, —C(═O)—, —O—C(═O)—O—, —C(═O)—NH—, —NH—, —NH—C(═NH)— (H may be substituted with a substituent such as an alkyl group, an acyl group, or the like), —S—, —S(═O)₂—, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²— [in the formulae, Y²¹ and Y²² each independently represent a divalent hydrocarbon group which may have a substituent, O represents an oxygen atom, and m″ represents an integer of 0 to 3].

In a case where the divalent linking group having a hetero atom is —C(═O)—NH—, —C(═O)—NH—C(═O)—, —NH—, or —NH—C(═NH)—, H may be substituted with a substituent such as an alkyl group or an acyl group. The substituent (an alkyl group, an acyl group, or the like) has preferably 1 to 10 carbon atoms, more preferably 1 to 8 carbon atoms, and particularly preferably 1 to 5 carbon atoms.

In General Formulae —Y²¹—O—Y²²—, —Y²¹—O—, —Y²¹—C(═O)—O—, —C(═O)—O—Y²¹—, —[Y²¹—C(═O)—O]_{m″}—Y²—, —Y²¹—O—C(═O)—Y²—, or —Y”-S(═O)₂—O—Y²—, Y²¹ and Y² each independently represent a divalent hydrocarbon group which may have a substituent. Examples of the divalent hydrocarbon group include those described as the divalent linking group as Ya^{x1} (the divalent hydrocarbon group which may have a substituent).

Y²¹ represents preferably a linear aliphatic hydrocarbon group, more preferably a linear alkylene group, still more preferably a linear alkylene group having 1 to 5 carbon atoms, and particularly preferably a methylene group or an ethylene group.

Y²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²—C(═O)—O]^{m″}—Y²—, m″ represents an integer of 0 to 3, preferably an integer of 0 to 2, more preferably 0 or 1, and particularly preferably 1. That is, a group represented by Formula —Y²¹—C(═O)—O—Y²²— is particularly preferable as the group represented by Formula —[Y²¹—C(═O)—O]_{m″}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Among these, Va^{x0} represents preferably a single bond, an ester bond [—C(═O)—O—or —O—C(═O)—], an ether bond (—O—), a linear or branched alkylene group, or a combination thereof, more preferably a single bond or an ester bond [—C(═O)—O—or —O—C(═O)—], and still more preferably a single bond.

In Formula (a0-1), Wa represents a divalent aromatic hydrocarbon group which may have a substituent.

Examples of the aromatic hydrocarbon group as Wa include a group in which two hydrogen atoms have been removed from an aromatic ring. The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 10 carbon atoms. Specific examples of the aromatic ring include aromatic hydrocarbon rings such as benzene, naphthalene, anthracene, and phenanthrene; and an aromatic heterocyclic ring obtained by substituting some of the carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Further, examples of the aromatic hydrocarbon group as Wa include a group in which two hydrogen atoms have been removed from an aromatic compound (such as biphenyl or fluorene) having two or more aromatic rings.

Among these, Wa represents preferably a group in which two hydrogen atoms have been removed from benzene, naphthalene, anthracene, or biphenyl, more preferably a group (that is, a phenylene group or a naphthylene group) in which two hydrogen atoms have been removed from benzene or naphthalene, and still more preferably a group (that is, a phenylene group) in which two hydrogen atoms have been removed from benzene.

Examples of the substituent that the divalent aromatic hydrocarbon group as Wa may have include a linear or branched alkyl group having 1 to 5 carbon atoms, a halogen atom, and a linear or branched halogenated alkyl group having 1 to 5 carbon atoms. Examples of the linear or branched alkyl group having 1 to 5 carbon atoms include a methyl group, an ethyl group, a propyl group, an isopropyl group, an n-butyl group, an isobutyl group, a tert-butyl group, a pentyl group, an isopentyl group, and a neopentyl group. Among these, a methyl group is preferable.

Examples of the halogen atom include a fluorine atom, a chlorine atom, a bromine atom, and an iodine atom. Among these, a fluorine atom is preferable.

The halogenated alkyl group having 1 to 5 carbon atoms is a group in which some or all of hydrogen atoms in the alkyl group having 1 to 5 carbon atoms have been substituted with halogen atoms.

In Formula (a0-1), Va⁰ represents a divalent hydrocarbon group which may have an ether bond. The divalent hydrocarbon group as Va⁰ may be an aliphatic hydrocarbon group or an aromatic hydrocarbon group.

The aliphatic hydrocarbon group as the divalent hydrocarbon group represented by Va^{a} may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Examples of the aliphatic hydrocarbon group containing a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of a linear or branched aliphatic hydrocarbon group. Examples of the linear or branched aliphatic hydrocarbon group include the same one as the above-described linear aliphatic hydrocarbon group or the above-described branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va⁰ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms. Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some of the carbon atoms constituting the above-described aromatic hydrocarbon rings have been substituted with hetero atoms. Examples of the hetero atom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (an alkyl chain in the arylalkyl group) has preferably 1 to 4 carbon atoms, more preferably 1 or 2 carbon atoms, and particularly preferably 1 carbon atom.

In Formula (a0-1), nₐ₀ represents an integer of 0 to 2, preferably 0 or 1, and more preferably 0.

In General Formula (a0-1), Ra⁰⁰ represents an acid dissociable group.

Here, “acid dissociable group” indicates both a group (i) having an acid dissociation property in which a bond between the acid dissociable group and an atom adjacent to the acid dissociable group can be cleaved due to the action of an acid; and a group (ii) in which some bonds are cleaved due to the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, relatively, the solubility in a developing solution is changed such that the solubility is increased in a case where the developing solution is an alkali developing solution and the solubility is decreased in a case where the developing solution is an organic developing solution.

Examples of the acid dissociable group as Ra⁰⁰ include those which have been suggested as the acid dissociable groups of the base resin for a chemically amplified resist composition.

Specific suggested examples of the acid dissociable group of the base resin for a chemically amplified resist composition include “acetal type acid dissociable group” and “tertiary alkyl ester type acid dissociable group” described below.

### Acetal Type Acid Dissociable Group:

Examples of the acid dissociable group as Ra⁰⁰ include an acid dissociable group represented by General Formula (a1-r-1) (hereinafter, also referred to as “acetal type acid dissociable group”).

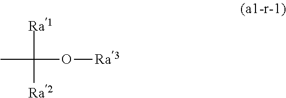

[In the formula, Ra′¹ and Ra′² represent a hydrogen atom or an alkyl group, Ra′³ represents a hydrocarbon group, and Ra′³ may be bonded to any one of Ra′¹ or Ra′² to form a ring.]

In Formula (a1-r-1), it is preferable that at least one of Ra′¹ or Ra′² represents a hydrogen atom and more preferable that both Ra′¹ and Ra′² represent a hydrogen atom. In a case where Ra′¹ or Ra′² represents an alkyl group, examples of the alkyl group include the same alkyl groups exemplified as the substituent which may be bonded to the carbon atom at the α-position in the description on the α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 10 carbon atoms.

Specific examples of the aromatic ring include aromatic hydrocarbon rings such as benzene, naphthalene, anthracene, and phenanthrene; and aromatic heterocyclic rings in which some of the carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring, a thiophene ring, and a furan ring.

Specific examples of the aromatic hydrocarbon group as Ra′³ include a group in which one hydrogen atom has been removed from the above-described aromatic hydrocarbon ring or aromatic heterocyclic ring (an aryl group or a heteroaryl group), a group in which one hydrogen atom has been removed from an aromatic compound having two or more aromatic rings (such as biphenyl or fluorene), and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

In Formula (a1-r-1), the hydrocarbon group as Ra′³ is preferably a cyclic aliphatic hydrocarbon group, more preferably an aliphatic hydrocarbon group which is a polycyclic group, still more preferably a group in which one hydrogen atom has been removed from adamantane, norbornane, isobornane, tricyclodecane, or tetracyclododecane, and particularly preferably a group in which one hydrogen atom has been removed from adamantane.

### Tertiary Alkyl Ester Type Acid Dissociable Group:

Examples of the acid dissociable group as Ra⁰⁰ include an acid dissociable group represented by General Formula (a1-r-2).

Among the acid dissociable groups represented by General Formula (a1-r-2), for convenience, a group formed of alkyl groups is referred to as “tertiary alkyl ester type acid dissociable group”.

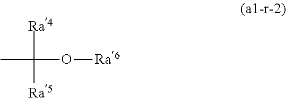

[In the formula, Ra′⁴ to Ra′⁶ each independently represent a hydrocarbon group which may have a substituent, and Ra′⁵ and Ra′⁶ may be bonded to each other to form a ring.]

Examples of the hydrocarbon group which may have a substituent as Ra′⁴ to Ra′⁶ include a linear or branched alkyl group (chain-like alkyl group), a linear or branched alkenyl group (chain-like alkenyl group), and a cyclic hydrocarbon group.

The linear alkyl group as Ra′⁴ to Ra′⁶ has preferably 1 to 20 carbon atoms, more preferably 1 to 15 carbon atoms, and most preferably 1 to 10 carbon atoms. Specific examples thereof include a methyl group, an ethyl group, a propyl group, a butyl group, a pentyl group, a hexyl group, a heptyl group, an octyl group, a nonyl group, a decyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a henicosyl group, and a docosyl group.

The branched alkyl group as Ra′⁴ to Ra′⁶ has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

The linear or branched alkenyl group as Ra′⁴ to Ra′⁶ has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (an allyl group), and a butynyl group. Examples of the branched alkenyl group include a 1-methylpropenyl group and a 2-methylpropenyl group.

The chain-like alkyl group or the chain-like alkenyl group as Ra′⁴ to Ra′⁶ may have a substituent. Examples of the substituent that the chain-like alkyl group or the chain-like alkenyl group as Ra′⁴ to Ra′⁶ may have include a hydroxy group and an ether bond (—O—).

The cyclic hydrocarbon group as Ra′⁴ to Ra′⁶ may be a polycyclic group or a monocyclic group. Further, the cyclic hydrocarbon group as Ra′⁴ to Ra′⁶ may be an alicyclic hydrocarbon group or a fused cyclic group in which an aromatic ring is fused with an alicyclic hydrocarbon group.

The aliphatic hydrocarbon group which is a monocyclic group is preferably a group in which one hydrogen atom has been removed from a monocycloalkane or a monocycloalkene. The monocycloalkane has preferably 3 to 6 carbon atoms, and specific examples thereof include cyclopentane and cyclohexane. The monocycloalkene preferably has 3 to 6 carbon atoms, and specific examples thereof include cyclopentene and cyclohexene.

The aliphatic hydrocarbon group which is a polycyclic group is preferably a group in which one hydrogen atom has been removed from a polycycloalkane or a polycycloalkene. The polycycloalkane preferably has 7 to 12 carbon atoms, and specific examples thereof include adamantane, norbornane, isobornane, tricyclodecane, and tetracyclododecane. In addition, the polycycloalkene is preferably a group having 7 to 12 carbon atoms. Specific examples thereof include adamantene, norbornene, isobornene, tricyclodecene, and tetracyclododecene.

Examples of the fused cyclic group in which an aromatic ring is fused with an alicyclic hydrocarbon group include a group in which one hydrogen atom has been removed from an aliphatic ring of a bicyclic compound such as tetrahydronaphthalene or indane. In the cyclic aliphatic hydrocarbon group as Ra′⁴ to Ra′⁶, some of the carbon atoms constituting the ring structure thereof may be substituted with a substituent having a hetero atom. As the substituent having a hetero atom, —O—, —C(═O)—O—, —S—, —S(═O)₂—, or —S(═O)₂—O— is preferable.

The aromatic hydrocarbon group as Ra′⁴ to Ra′⁶ is a hydrocarbon group having an aromatic ring. The aromatic hydrocarbon group has preferably 3 to 30 carbon atoms, more preferably 5 to 30, still more preferably 5 to 20, particularly preferably 6 to 15, and most preferably 6 to 10. Here, the number of carbon atoms in a substituent is not included in the number of carbon atoms. Specific examples of the aromatic ring in the aromatic hydrocarbon group as Ra′⁴ to Ra′⁶ include benzene, fluorene, naphthalene, anthracene, phenanthrene, biphenyl, and an aromatic heterocyclic ring obtained by substituting some of the carbon atoms constituting one of these aromatic rings with hetero atoms. Examples of the hetero atom in the aromatic heterocyclic rings include an oxygen atom, a sulfur atom, and a nitrogen atom. Specific examples of the aromatic heterocyclic ring include a thiophene ring, a furan ring, and a pyridine ring.

Specific examples of the aromatic hydrocarbon group as Ra′⁴ to Ra′⁶ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (an alkyl chain in the arylalkyl group) has preferably 1 to 4 carbon atoms, more preferably 1 or 2 carbon atoms, and particularly preferably 1 carbon atom.

The cyclic hydrocarbon group as Ra′⁴ to Ra′⁶ may include a substituent. Examples of the substituent that the cyclic hydrocarbon group as Ra′⁴ to Ra′⁶ may have include a halogen atom (such as a fluorine atom, a chlorine atom, a bromine atom, or an iodine atom), —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra⁰⁶”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a divalent chain-like saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Here, some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of a single kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6] decanyl group, a tricyclo[3.3.1.13,7]decanyl group, a tetracyclo[6.2.1.13,6.02,7] dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In Formula (a1-r-2), in a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ in Formula (a1-r-2) represent an independent hydrocarbon group without being bonded to each other, suitable examples thereof include a group represented by Formula (a1-r2-4).

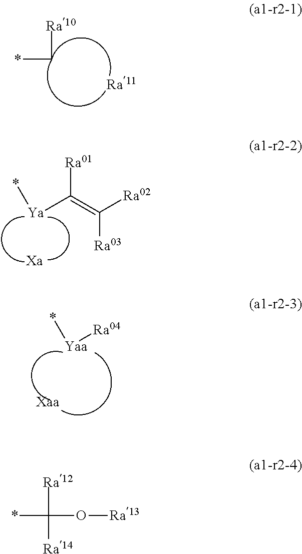

[In Formula (a1-r2-1), Ra′¹⁰ represents an alkyl group having 1 to 10 carbon atoms, and Ra′¹¹ represents an alicyclic hydrocarbon group formed with the carbon atom to which Ra′¹⁰ is bonded or a fused cyclic group having an aromatic ring fused with an alicyclic hydrocarbon group. In Formula (a1-r2-2), Ya represents a carbon atom. Xa represents a group that forms a cyclic hydrocarbon group with Ya. Some or all hydrogen atoms in this cyclic hydrocarbon group may be substituted. Ra⁰′ to Ra⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra⁰¹ to Ra⁰³ may be bonded to each other to form a cyclic structure. In Formula (a1-r2-3), Yaa represents a carbon atom. Xaa represents a group that forms an aliphatic cyclic group with Yaa. Ra⁰⁴ represents an aromatic hydrocarbon group which may have a substituent. In Formula (a1-r2-4), Ra′¹² and Ra′¹³ each independently represent a chain-like monovalent saturated hydrocarbon group having 1 to 10 carbon atoms or a hydrogen atom. Some or all hydrogen atoms in this chain-like saturated hydrocarbon group may be substituted. Ra′¹⁴ represents a hydrocarbon group which may have a substituent. * represents a bonding site (the same applies hereinafter).]

In Formula (a1-r2-1), preferred examples of the alkyl group having 1 to 10 carbon atoms as Ra′¹⁰ include a linear or branched alkyl group as Ra′³ in Formula (a1-r-1) and the groups described as the aliphatic hydrocarbon group of a polycyclic group or a monocyclic group. It is preferable that Ra′¹⁰ represents an alkyl group having 1 to 5 carbon atoms.

In Formula (a1-r2-1), as the alicyclic hydrocarbon group formed by Ra′¹¹ together with the carbon atom to which Ra′¹⁰ is bonded or the fused cyclic group having an aromatic ring fused with an alicyclic hydrocarbon group, the groups described as the cyclic hydrocarbon group as Ra′⁴ to Ra′⁶ in Formula (a1-r-2) are preferable, a group in which one hydrogen atom has been removed from a monocycloalkane or a polycycloalkane is more preferable, a group in which one hydrogen atom has been removed from cyclopentane, cyclohexane, adamantane, norbornane, isobornane, tricyclodecane, or tetracyclododecane is still more preferable, and a group in which one hydrogen atom has been removed from cyclopentane or adamantane is particularly preferable.

In the alicyclic hydrocarbon group formed by Ra′¹¹ together with the carbon atom to which Ra′¹⁰ is bonded, some of the carbon atoms constituting the ring structure thereof may be substituted with a substituent containing a hetero atom. As the substituent having a hetero atom, —O—, —C(═O)—O—, —S—, —S(═O)₂—, or —S(═O)₂—O— is preferable.

In Formula (a1-r2-2), as the cyclic hydrocarbon group formed by Xa together with Ya, the groups described as the cyclic hydrocarbon group as Ra′⁴ to Ra′⁶ in Formula (a1-r-2) are preferable, a group in which one hydrogen atom has been removed from a monocycloalkane is more preferable, a group in which one hydrogen atom has been removed from cyclopentane or cyclohexane is still more preferable, and a group in which one hydrogen atom has been removed from cyclopentane is particularly preferable.

The cyclic hydrocarbon group that is formed by Xa together with Ya may have a substituent. Examples of the substituent include the same group described as the substituent that the cyclic hydrocarbon group as Ra′⁴ to Ra′⁶ in Formula (a1-r-2) may have.

In General Formula (a1-r2-2), examples of the chain-like monovalent saturated hydrocarbon group having 1 to 10 carbon atoms as Ra⁰¹ to Ra⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra⁰¹ to Ra⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group, and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

Among these, from the viewpoint of easily synthesizing a monomer compound from which the constitutional unit (a0) is derived, Ra⁰¹ to Ra⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Some or all hydrogen atoms in the chain-like saturated hydrocarbon group and the aliphatic cyclic saturated hydrocarbon group represented by Ra⁰¹ to Ra⁰³ may be substituted.

Examples of the group having a carbon-carbon double bond generated by two or more of Ra⁰¹ to Ra⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of easily synthesizing a monomer compound from which the constitutional unit (a0) is derived, a cyclopentenyl group, a cyclohexenyl group, or a cyclopentylidenethenyl group is preferable.

In Formula (a1-r2-3), as the aliphatic cyclic group formed by Xaa together with Yaa, the groups described as the cyclic hydrocarbon group as Ra′⁴ to Ra′⁶ in Formula (a1-r-2) is preferable, a group in which one hydrogen atom has been removed from a monocycloalkane is more preferable, and a group in which one hydrogen atom has been removed from cyclopentane or cyclohexane is still more preferable.

In Formula (a1-r2-3), examples of the aromatic hydrocarbon group as Ra⁰⁴ include a group in which one or more hydrogen atoms have been removed from an aromatic hydrocarbon ring having 5 to 30 carbon atoms. Among these, Ra⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, phenanthrene, thiophene, or furan, still more preferably a group one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent that Ra⁰⁴ in Formula (a1-r2-3) may have include a methyl group, an ethyl group, a propyl group, a hydroxyl group, a carboxyl group, a halogen atom (such as a fluorine atom, a chlorine atom, or a bromine atom), an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a chain-like monovalent saturated hydrocarbon group having 1 to 10 carbon atoms or a hydrogen atom. Examples of the chain-like monovalent saturated hydrocarbon group having 1 to 10 carbon atoms as Ra′¹² and Ra′¹³ include the same groups as those for the chain-like monovalent saturated hydrocarbon group having 1 to 10 carbon atoms as Ra⁰¹ to Ra⁰³. Some or all hydrogen atoms in this chain-like saturated hydrocarbon group may be substituted.

Among the examples, Ra′¹² and Ra′¹³ represent preferably a hydrogen atom or an alkyl group having 1 to 5 carbon atoms, more preferably an alkyl group having 1 to 5 carbon atoms, still more preferably a methyl group or an ethyl group, and particularly preferably a methyl group.

Some or all hydrogen atoms in the chain-like saturated hydrocarbon groups represented by Ra′¹² and Ra′¹³ may be substituted.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent that Ra′¹⁴ may have include the same groups as those for the substituent that Ra⁰⁴ may have.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

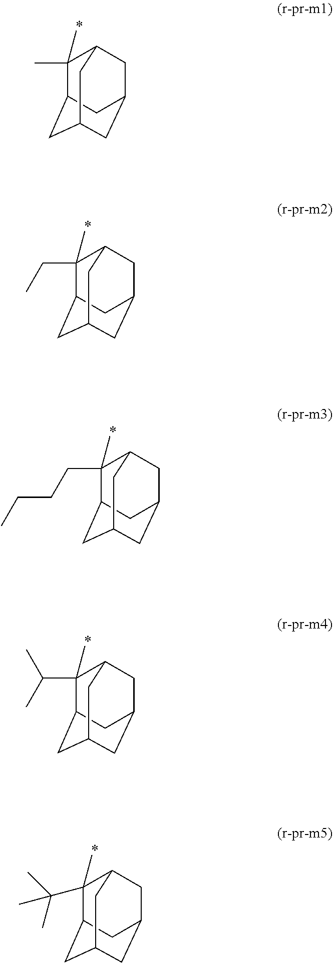

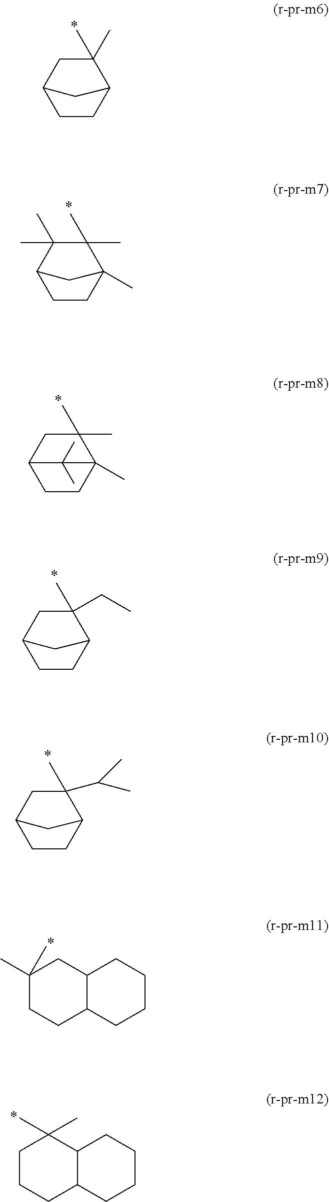

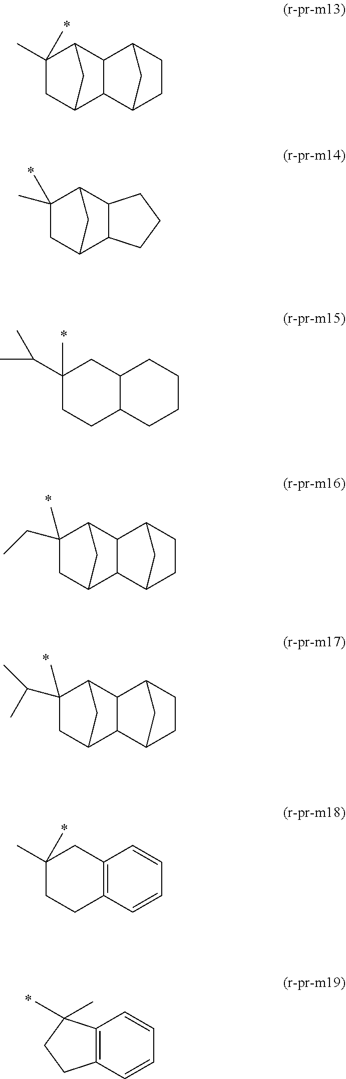

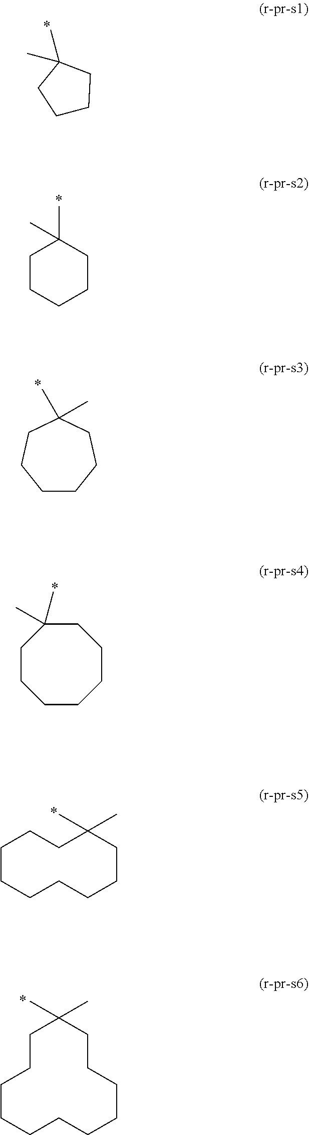

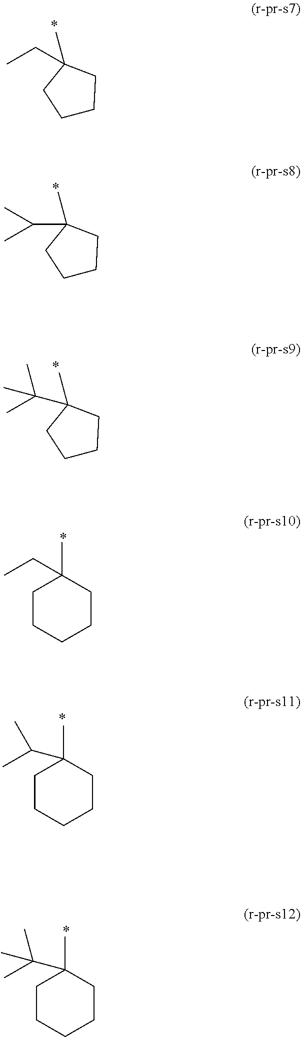

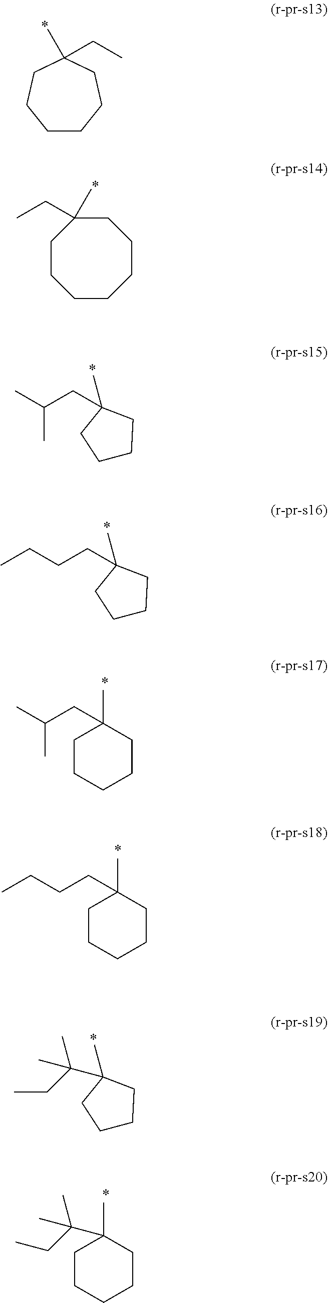

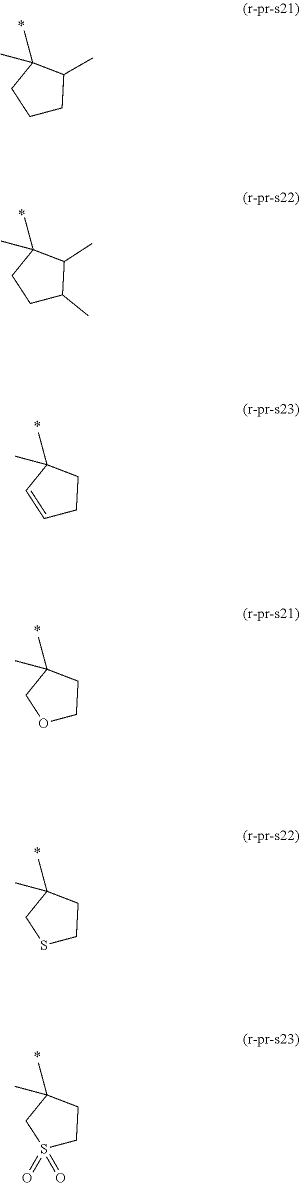

Specific examples of the group represented by Formula (a1-r2-2) are shown below.

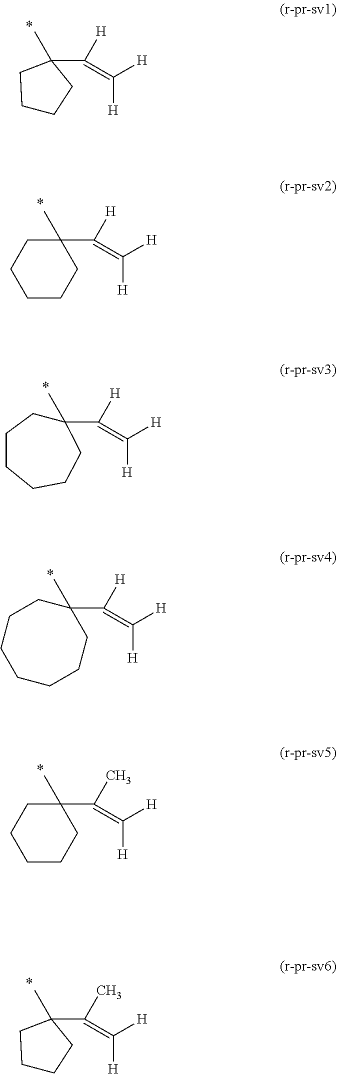

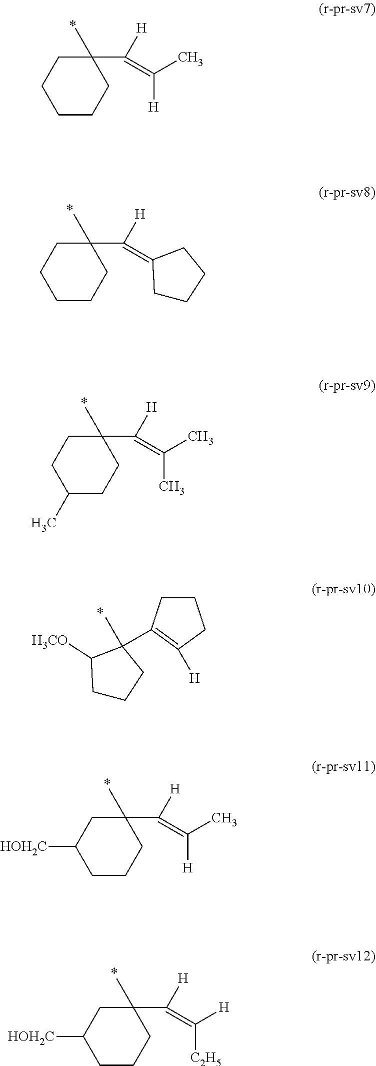

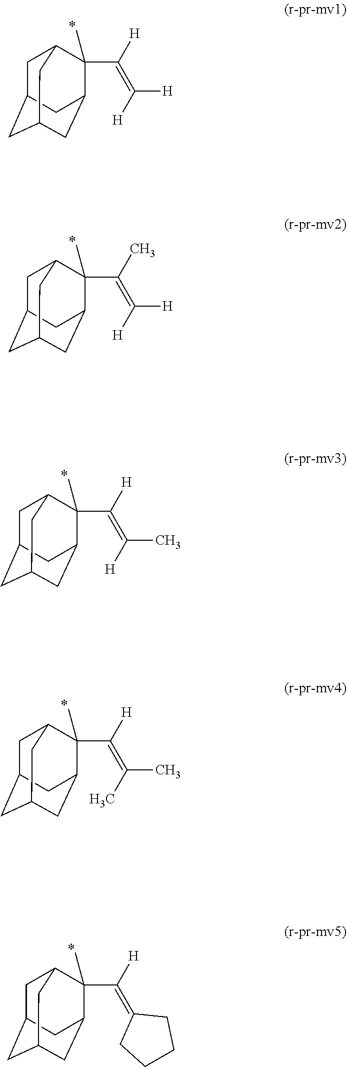

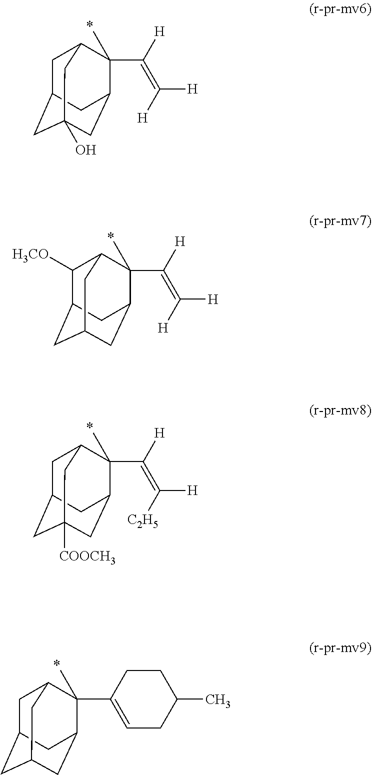

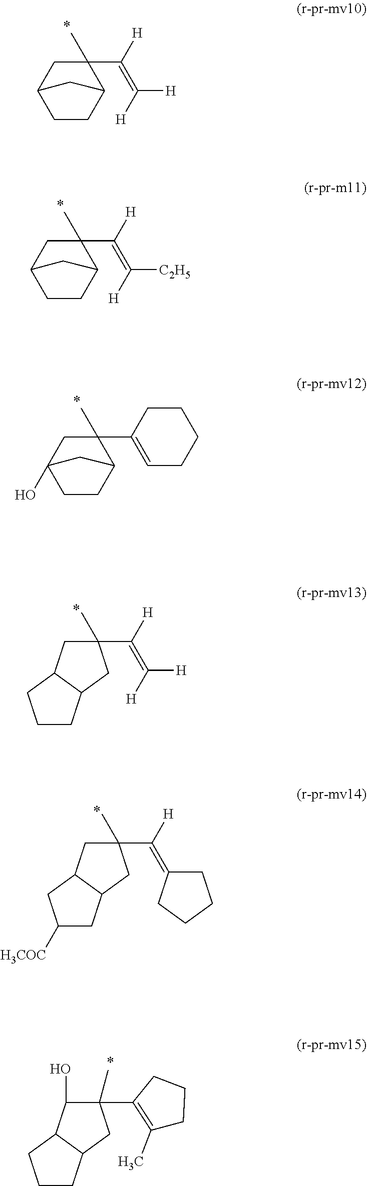

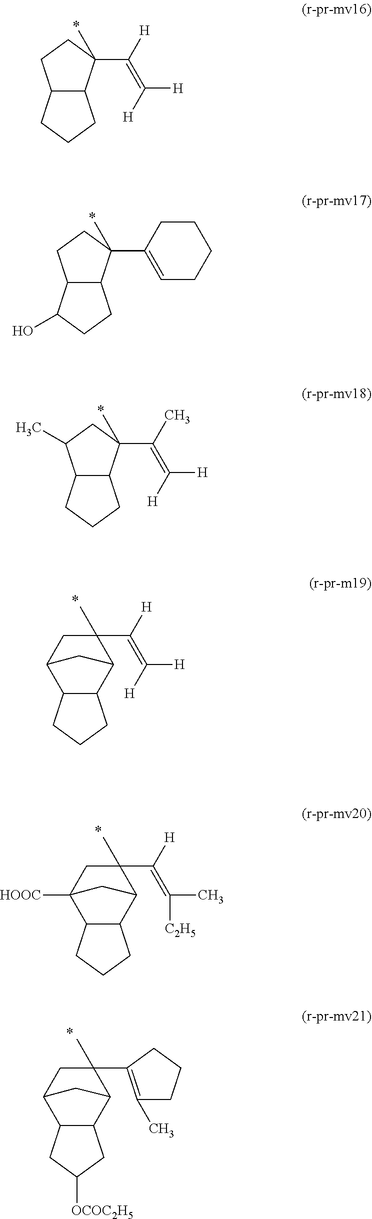

Specific examples of the group represented by Formula (a1-r2-3) are shown below.

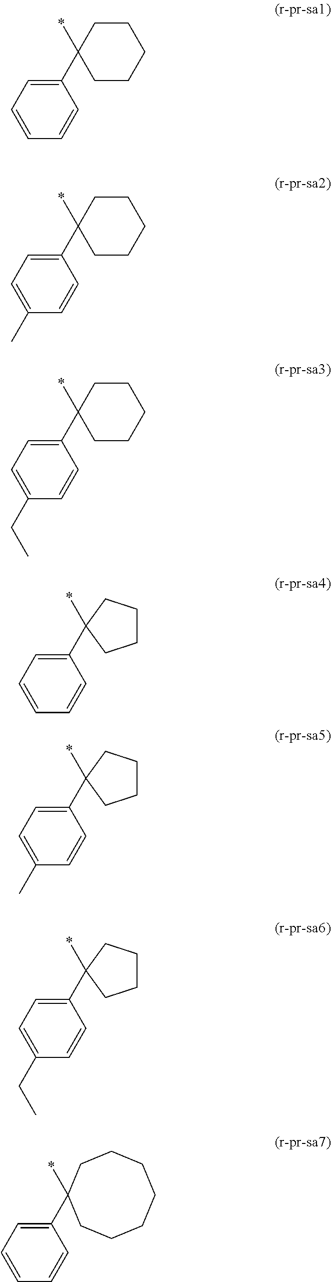

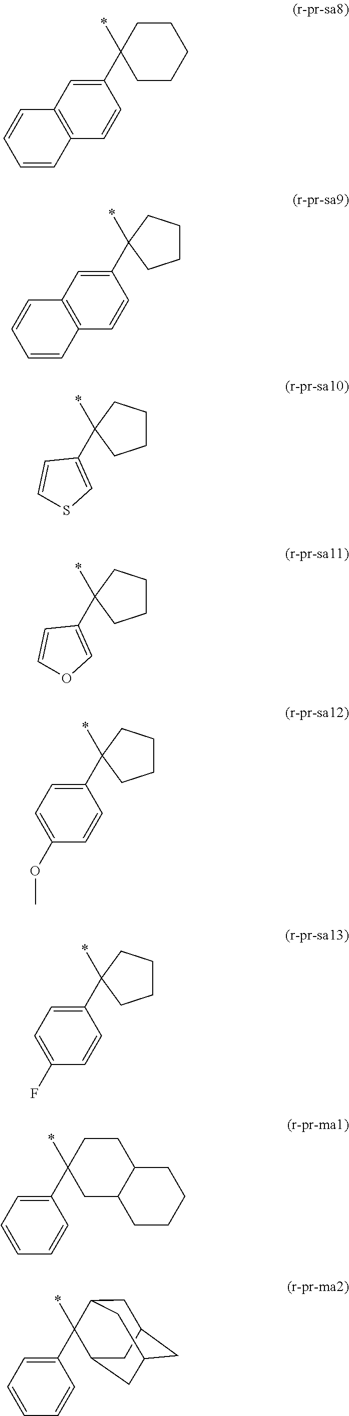

Specific examples of the group represented by Formula (a1-r2-4) are shown below.

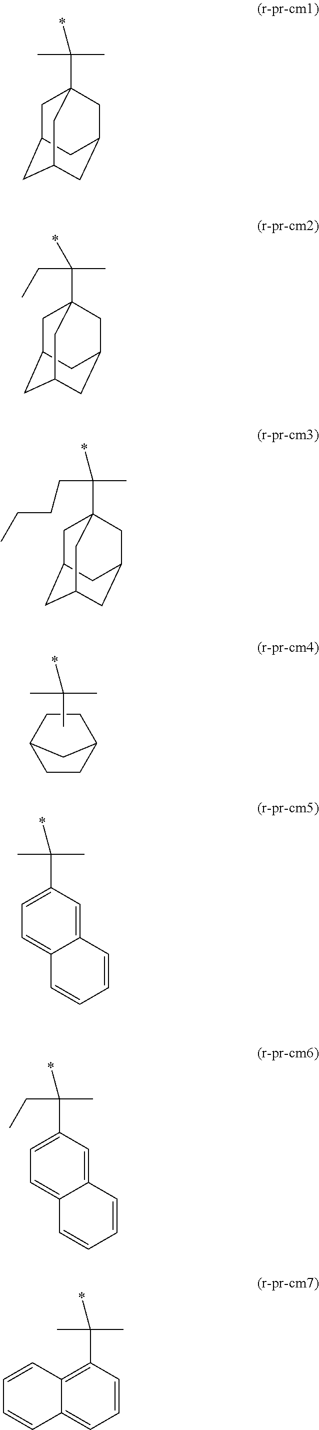

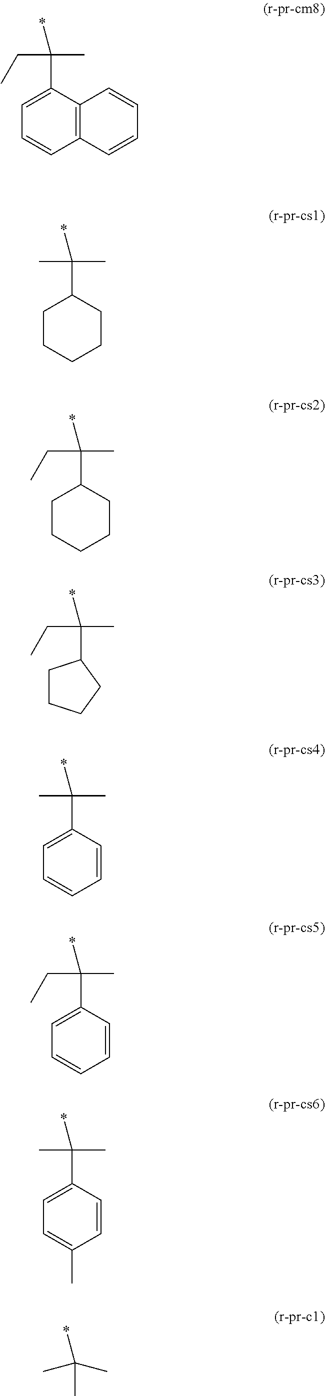

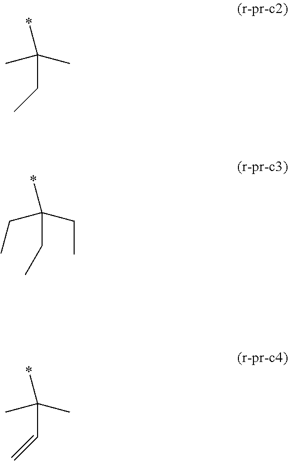

In Formula (a0-1), as the acid dissociable group as Ra⁰⁰, a group represented by any of Formulae (a1-r2-1) to (a1-r2-4) is preferable, and a group represented by Formula (a1-r2-2) or a group represented by Formula (a1-r2-3) is more preferable from the viewpoint that the etching resistance is likely to be further enhanced.

Specific examples of the constitutional unit (a0) are shown below. In the formulae, R⁰ has the same definition as described above.

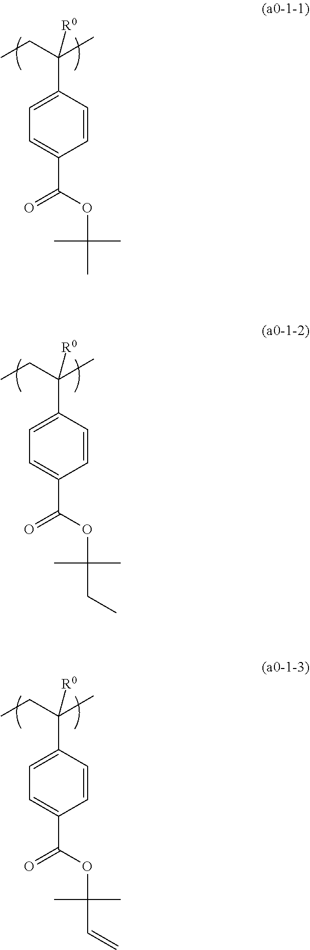

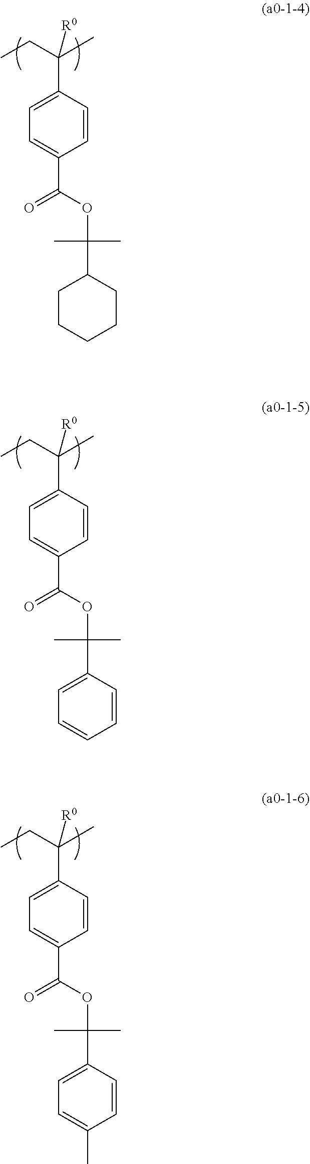

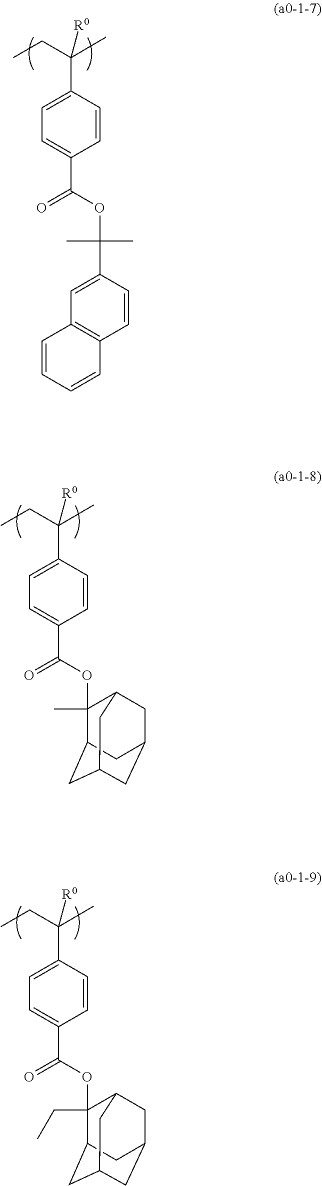

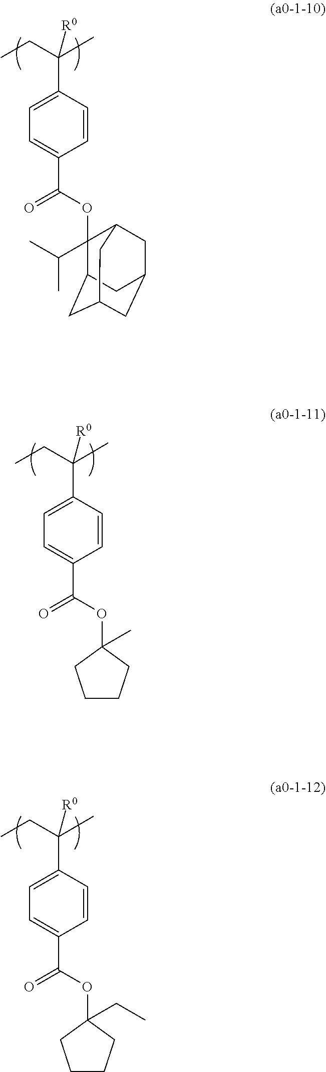

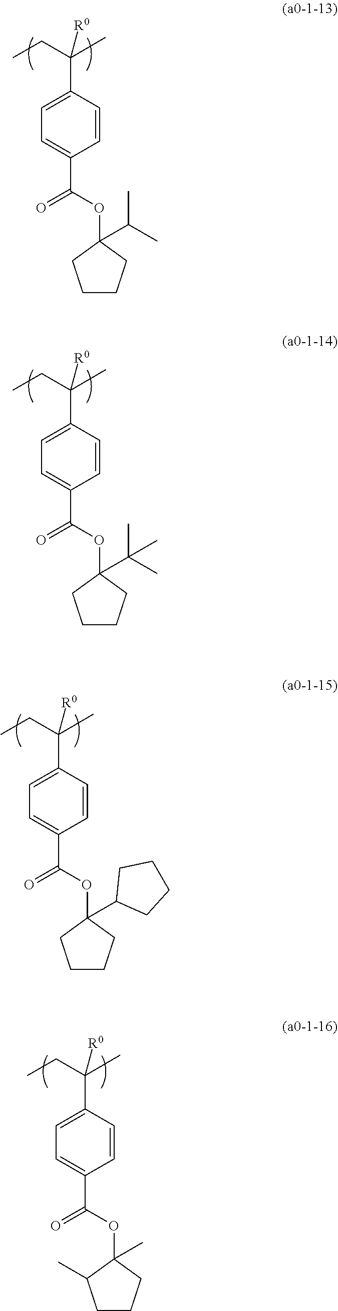

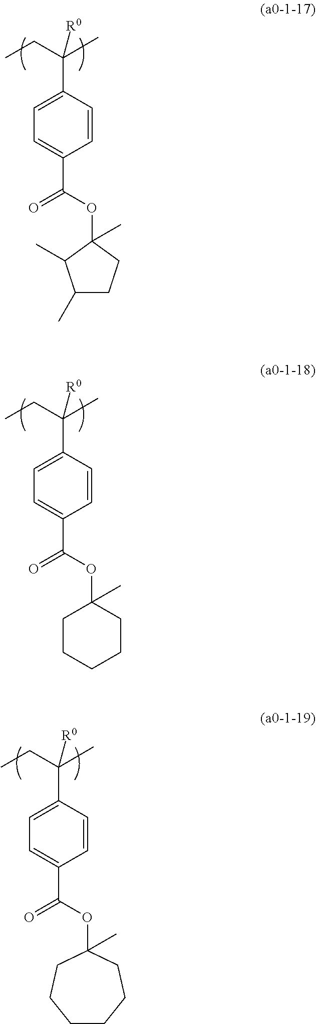

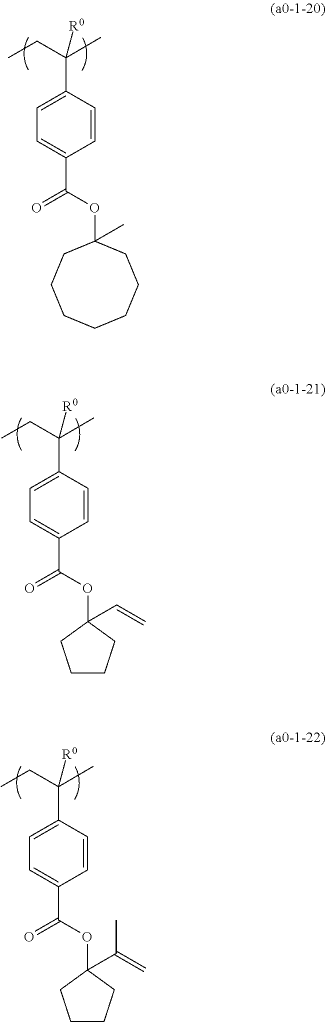

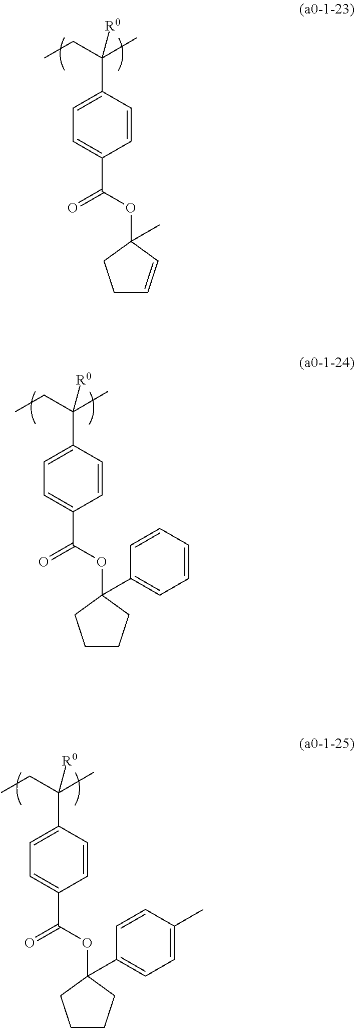

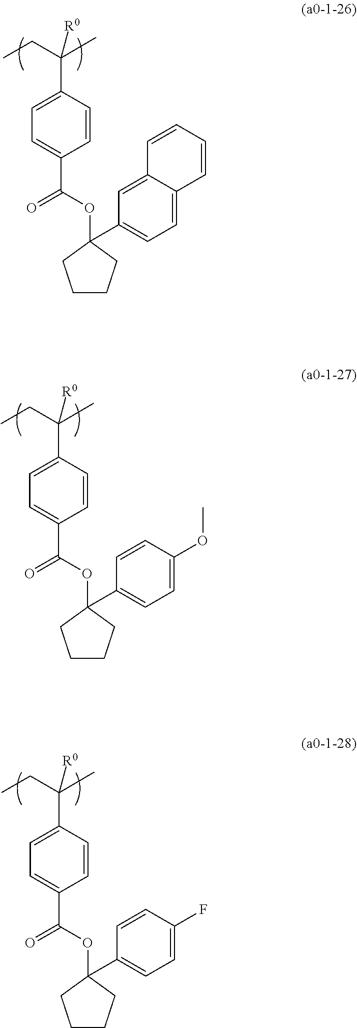

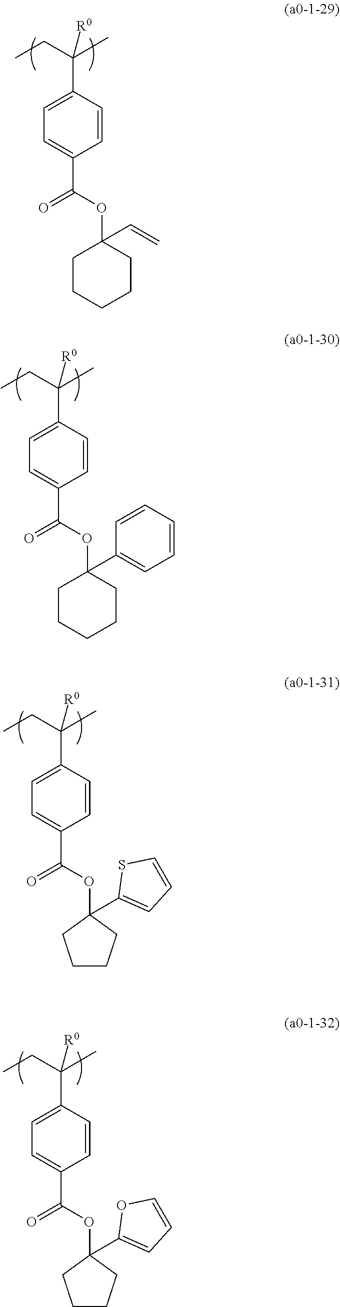

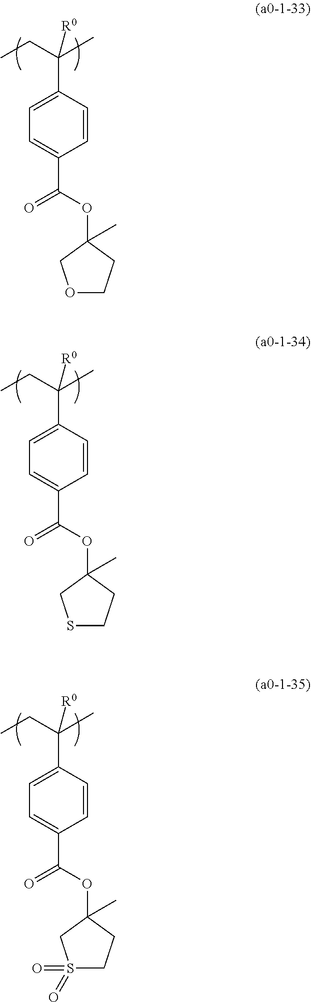

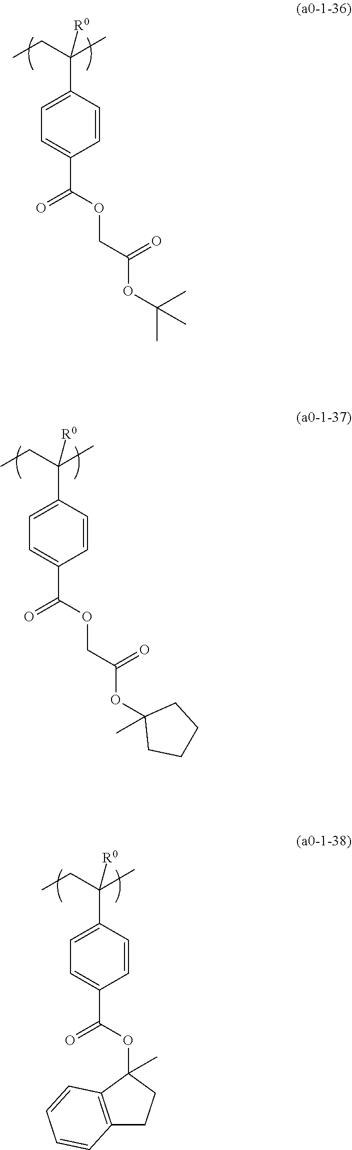

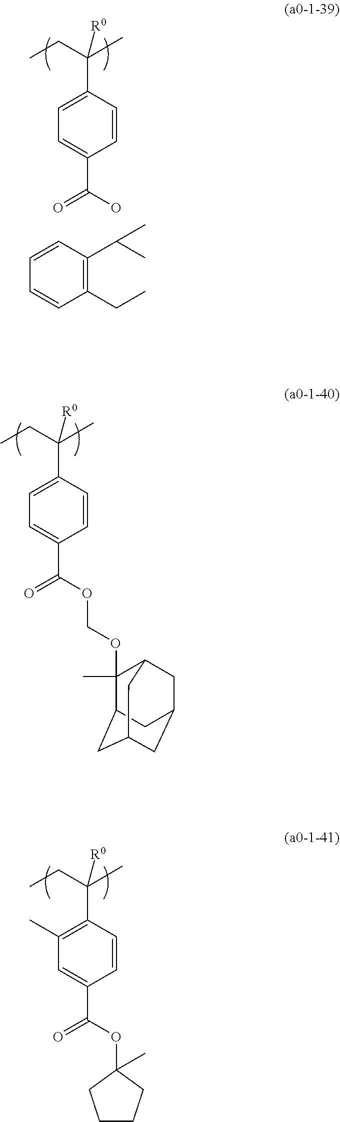

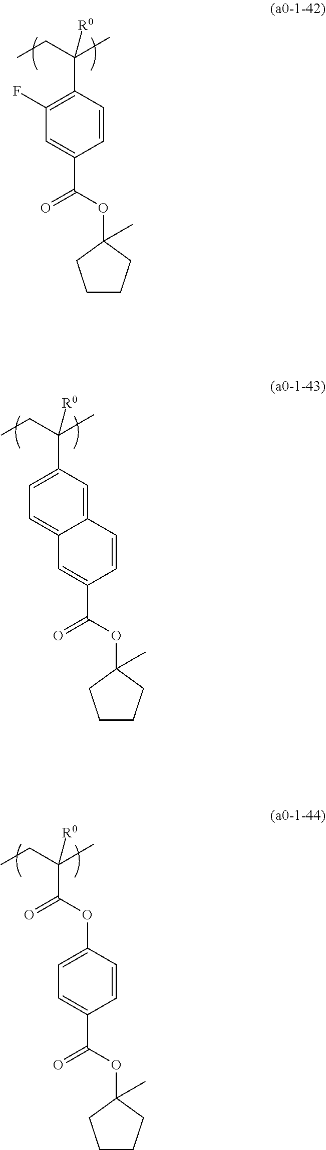

As the constitutional unit (a0), at least one selected from the group consisting of constitutional units each represented by Chemical Formulae (a0-1-1) to (a0-1-44) is preferable, and at least one selected from the group consisting of constitutional units each represented by Chemical Formulae (a0-1-8) to (a0-1-17), (a0-1-21) to (a0-1-26), (a0-1-38), (a0-1-39), and (a0-1-41) to (a0-1-43) is more preferable.

The constitutional unit (a0) contained in the component (A1) may be one kind or may be two or more kinds.

The proportion of the constitutional unit (a0) in the component (A1) is preferably in a range of 30% to 95% by mole, more preferably in a range of 40% to 85% by mole, and still more preferably in a range of 50% to 70% by mole with respect to the total amount (100% by mole) of all constitutional units constituting the component (A1).

In a case where the proportion of the constitutional unit (a0) is set to be greater than or equal to the lower limits, the sensitivity is likely to be increased. Meanwhile, in a case where the proportion thereof is set to be less than or equal to the upper limits, the characteristics such as the etching resistance and the effect of suppressing film reduction are further improved. Further, the balance between the constitutional unit (a0) and other constitutional units can be achieved.

### <<Other Constitutional Units>>

Such a component (A1) may have other constitutional unit as necessary in addition to the constitutional unit (a0).

Examples of other constitutional units include a constitutional unit (a10) represented by General Formula (a10-1), a constitutional unit containing an acid decomposable group whose polarity is increased due to an action of an acid (here, a constitutional unit corresponding to the constitutional unit (a0) is excluded), a constitutional unit (a2) containing a lactone-containing cyclic group, a —SO₂-containing cyclic group, or a carbonate-containing cyclic group, a constitutional unit (a3) containing a polar group-containing aliphatic hydrocarbon group (here, a constitutional unit corresponding to the constitutional unit (a1) or the constitutional unit (a2) is excluded), a constitutional unit (a4) containing an acid non-dissociable aliphatic cyclic group, and a constitutional unit derived from styrene or a derivative thereof.

In Regard to Constitutional Unit (a10):

The constitutional unit (a10) is a constitutional unit represented by General Formula (a10-1).

It is preferable that the component (A1) contained in the resist composition according to the present embodiment further has a constitutional unit (a10) represented by General Formula (a10-1) in addition to the constitutional unit (a0).

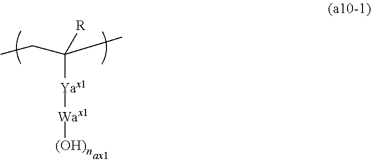

[In the formulae, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an (nₐₓ₁+1)-valent aromatic hydrocarbon group. nₐₓ₁ represents an integer of I or greater.]

In the Formula (a10-1), R represents a hydrogen atom, an alkyl group having 1 to 5 carbon atoms, or a halogenated alkyl group having 1 to 5 carbon atoms. R represents a hydrogen atom, an alkyl group having 1 to 5 carbon atoms, or a halogenated alkyl group having 1 to 5 carbon atoms.

As the alkyl group having 1 to 5 carbon atoms represented by R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms as R is a group in which some or all hydrogen atoms of the above-described alkyl group having 1 to 5 carbon atoms have been substituted with halogen atoms. Examples of the halogen atom include a fluorine atom, a chlorine atom, a bromine atom, and an iodine atom. Among these, a fluorine atom is particularly preferable.

R is preferably a hydrogen atom, an alkyl group having 1 to 5 carbon atoms, or a fluorinated alkyl group having 1 to 5 carbon atoms, and in terms of industrial availability, R is more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group.

In Formula (a10-1), the divalent linking group as Ya^{x1} is not particularly limited, and suitable examples thereof include a divalent hydrocarbon group which may have a substituent and a divalent linking group having a hetero atom. Examples of the divalent linking group as Ya^{x1} include the same groups as those for the divalent linking group as Vax^{x0} in Formula (a0-1).

Among these, Ya^{x1} represents preferably a single bond, an ester bond [—C(═O)—O—or —O—C(═O)—], an ether bond (—O—), a linear or branched alkylene group, or a combination thereof and more preferably a single bond or an ester bond [—C(═O)—O—or —O—C(═O)—].

In Formula (a10-1), Wa^{x1} represents an (nₐₓ₁+1)-valent aromatic hydrocarbon group.

Examples of the aromatic hydrocarbon group as Wa^{x1} include a group in which (nₐₓ₁+1) hydrogen atoms have been removed from an aromatic ring which may have a substituent. The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some of the carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} include a group in which (nₐₓ₁+1) hydrogen atoms have been removed from an aromatic compound (such as biphenyl or fluorene) having an aromatic ring which may have two or more substituents.

Among these, Wa^{x}t represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include those exemplified as the substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{a} represents a hydrogen atom, a methyl group, or a trifluoromethyl group.

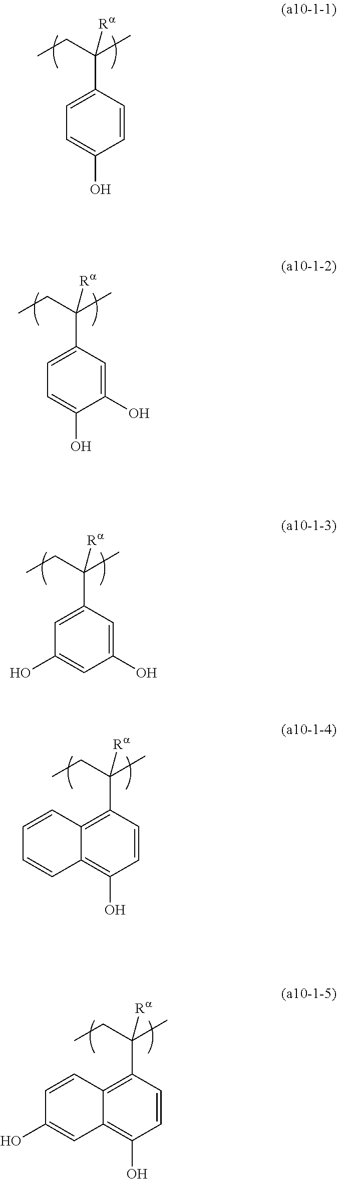

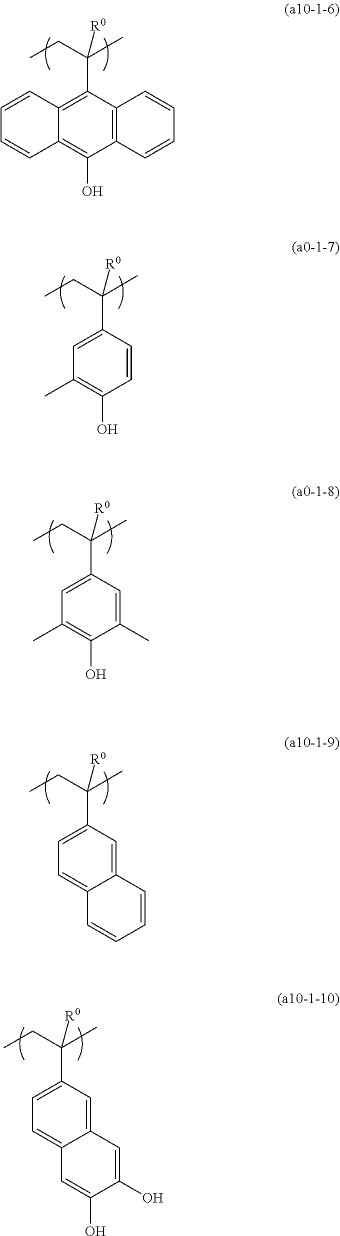

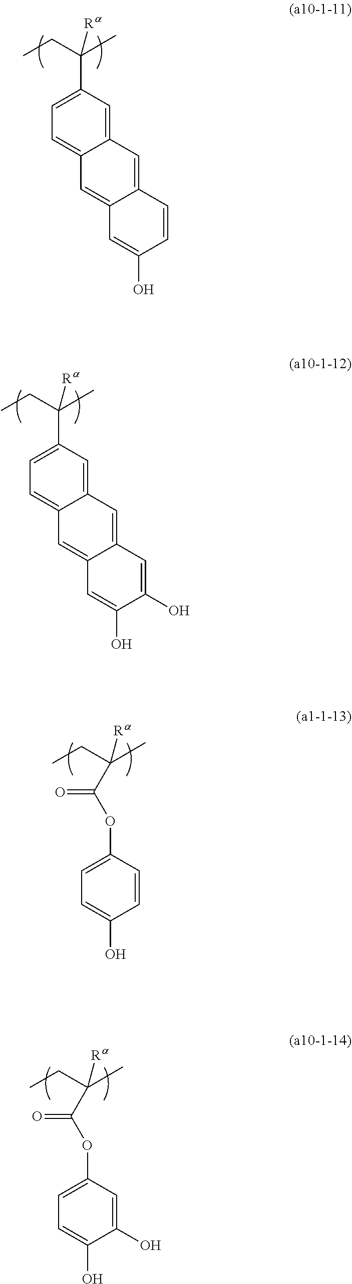

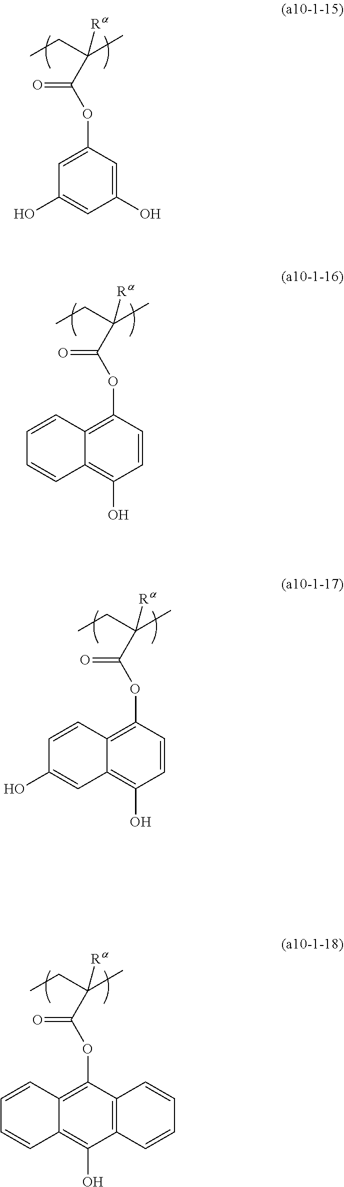

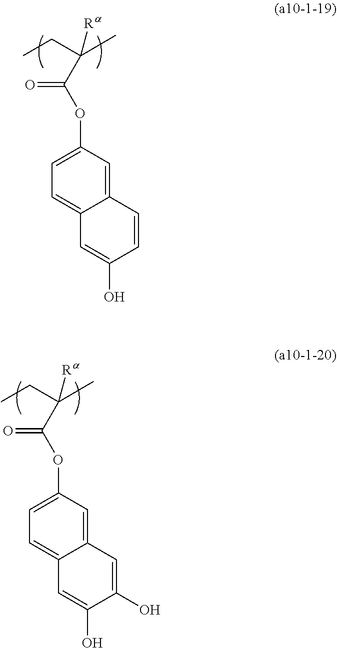

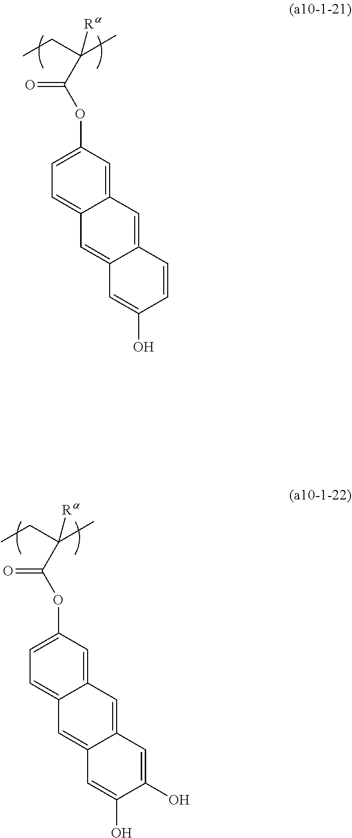

The constitutional unit (a10) included in the component (A1) may be used alone or two or more kinds thereof.

In a case where the component (A1) has the constitutional unit (a10), the proportion of the constitutional unit (a10) in the component (A1) is preferably in a range of 5% to 70% by mole, more preferably in a range of 15% to 60% by mole, and still more preferably in a range of 30% to 50% by mole with respect to the total amount (100% by mole) of all constitutional units constituting the component (A1).

In a case where the proportion of the constitutional unit (a10) is greater than or equal to the lower limits of the above-described preferable ranges, the etching resistance and the sensitivity are more likely to be increased. Further, in a case where the proportion of the constitutional unit (a10) is less than or equal to the upper limits of the above-described preferable ranges, the balance between the constitutional unit (a10) and the constitutional unit (a0) is likely to be achieved.

### In Regard to Constitutional Unit (a1):

The constitutional unit (a1) is a constitutional unit containing an acid decomposable group whose polarity is increased due to an action of an acid (here, a constitutional unit corresponding to the constitutional unit (a0) is excluded).

Examples of the acid decomposable group whose polarity is increased due to the action of an acid include groups which are decomposed due to the action of an acid to generate a polar group.

Examples of the polar group include a carboxy group, a hydroxyl group, an amino group, and a sulfo group (—SO₃H). Among these, a polar group containing —OH in the structure thereof (hereinafter, also referred to as “OH-containing polar group”) is preferable, a carboxy group or a hydroxyl group is more preferable, and a carboxy group is particularly preferable.

More specific examples of the acid decomposable group include a group (for example, a group obtained by protecting a hydrogen atom of the OH-containing polar group with an acid dissociable group) obtained by protecting the above-described polar group with an acid dissociable group.

Examples of the acid dissociable group are the same as those which have been suggested as the acid dissociable groups of the base resin for a chemically amplified resist composition.

Specific suggested examples of the acid dissociable group of the base resin for a chemically amplified resist composition include “acetal type acid dissociable group” and “tertiary alkyl ester type acid dissociable group” described as the acid dissociable group as R⁰⁰ in Formula (a0-1). Further, examples of the acid dissociable group here also include “tertiary alkyloxycarbonyl acid dissociable groups”.

### Tertiary Alkyloxycarbonyl Acid Dissociable Group:

Among the polar groups, examples of the acid dissociable group for protecting a hydroxyl group include an acid dissociable group represented by General Formula (a1-r-3) (hereinafter, also referred to as “tertiary alkyloxycarbonyl acid dissociable group” for convenience).

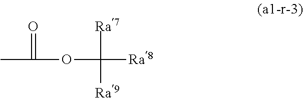

[In the formula, Ra′⁷ to Ra′⁹ each represent an alkyl group.]

In Formula (a1-r-3), Ra′⁷ to Ra′⁹ each independently represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable. Specific preferred examples of such a constitutional unit (a1) include constitutional units represented by General Formula (a1-1) or (a1-2) shown below.

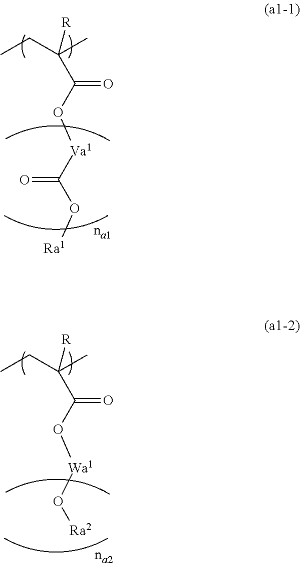

[In the formulae, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ra¹ represents an acid dissociable group represented by General Formula (a1-r-1) or (a1-r-2). Wa^{l} represents a (na₂+1)-valent hydrocarbon group. na₂ represents an integer of 1 to 3. Ra² represents an acid dissociable group represented by Formula (a1-r-1) or (a1-r-3).]

In Formula (a1-1), R has the same definition as that for R in Formula (a10-1).

In Formula (a1-1), Val has the same definition as that for Va⁰ in Formula (a0-1)

In Formula (a1-1), Ra¹ represents an acid dissociable group represented by Formula (a1-r-1) or (a1-r-2).

In Formula (a1-2), the (na₂+1)-valent hydrocarbon group as Wa¹ may be an aliphatic hydrocarbon group or an aromatic hydrocarbon group.

The aliphatic hydrocarbon group indicates a hydrocarbon group that has no aromaticity and may be saturated or unsaturated. In general, it is preferable that the alinhatic hydrocarbon group is saturated. Examples of the alinhatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of (na₂+1) is preferably divalent to tetravalent and more preferably divalent or trivalent.

Specific examples of the constitutional unit represented by Formula (a1-1) are shown below. In the formulae shown below, R^{α} represents a hydrogen atom, a methyl group, or a trifluoromethyl group.

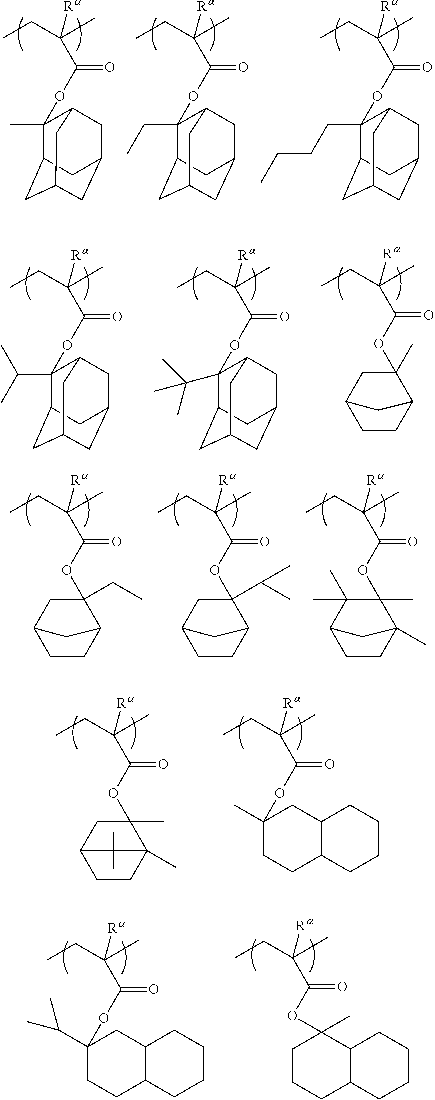

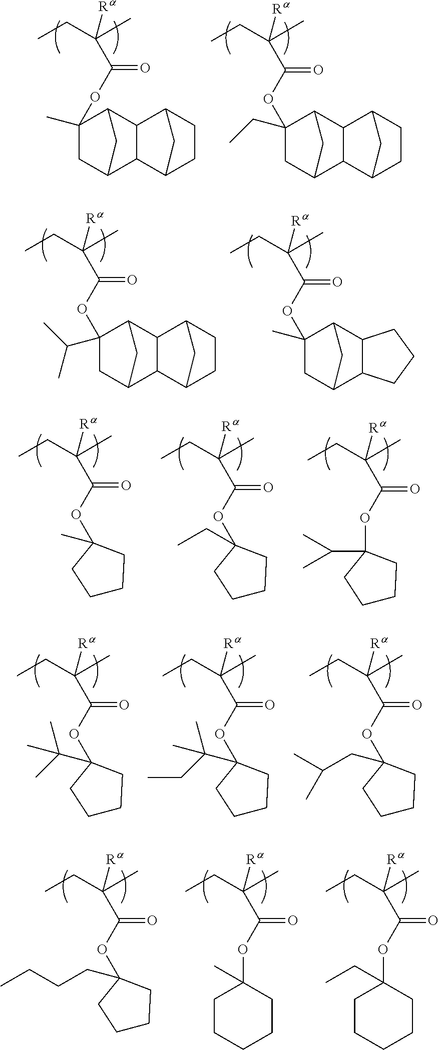

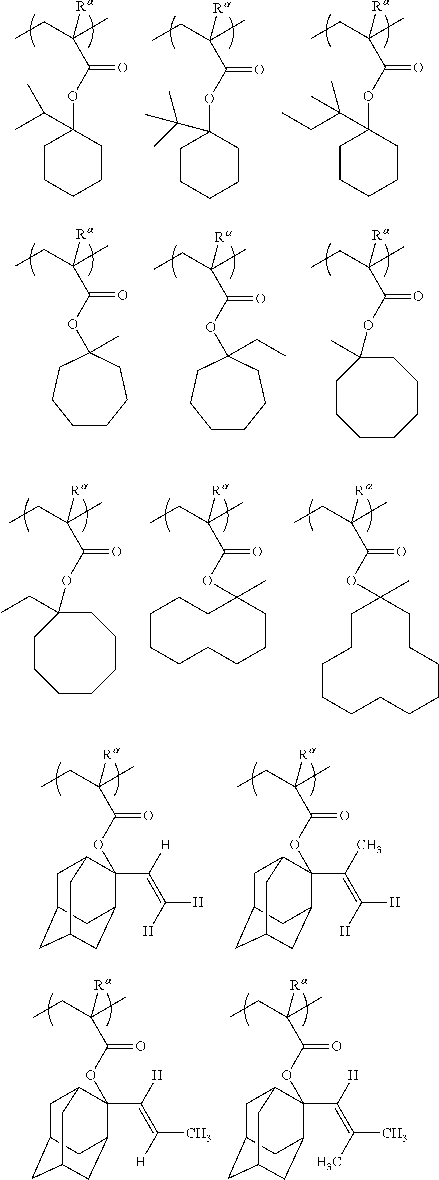

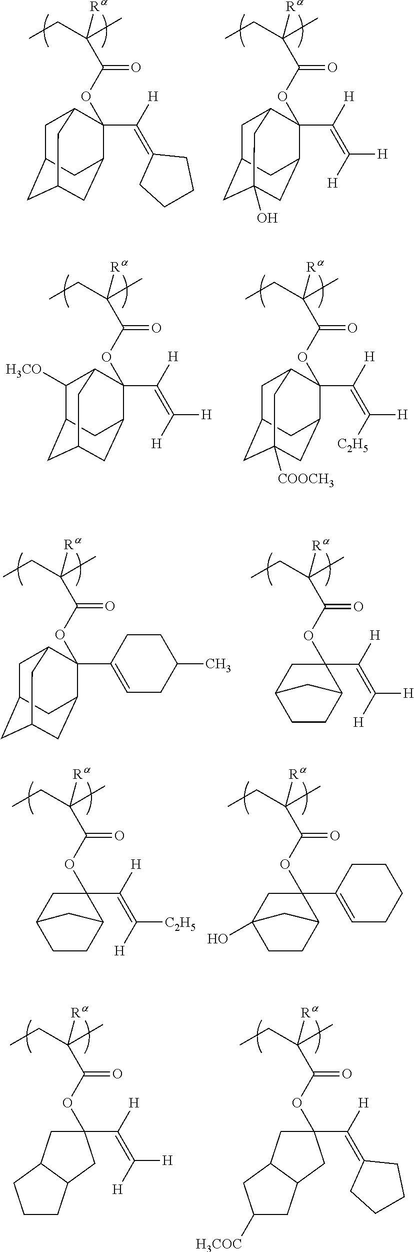

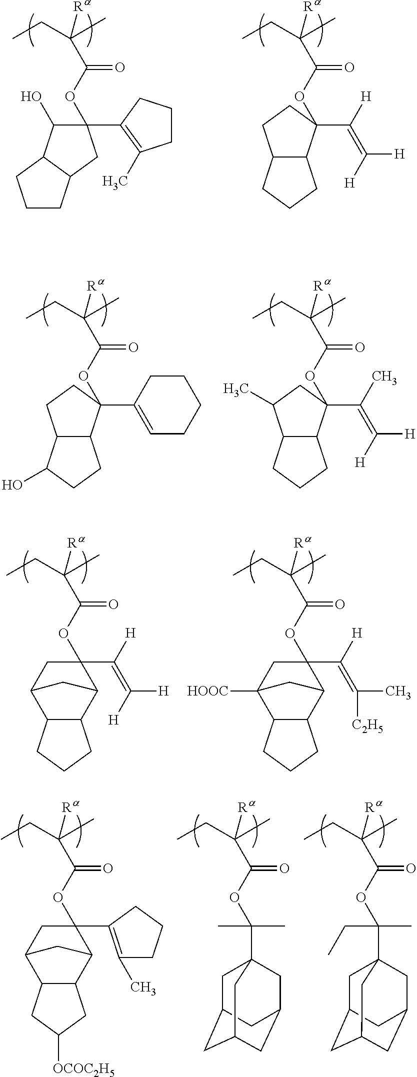

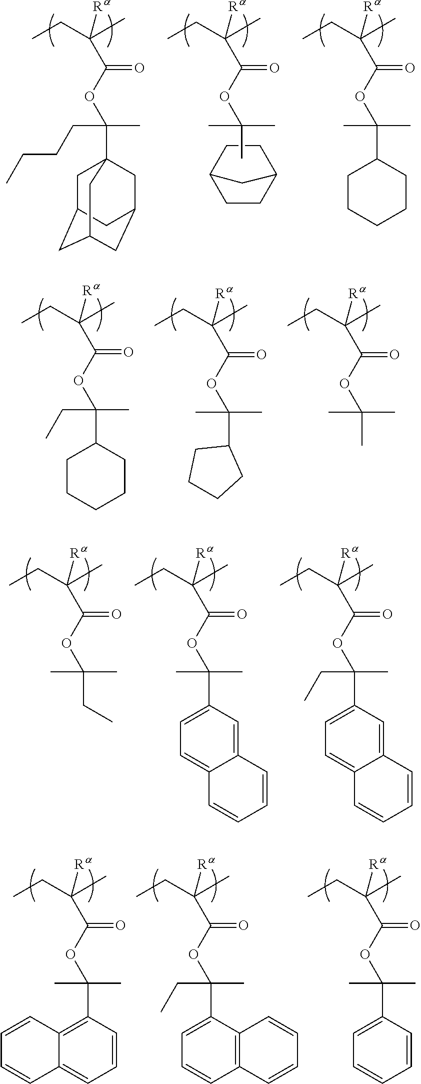

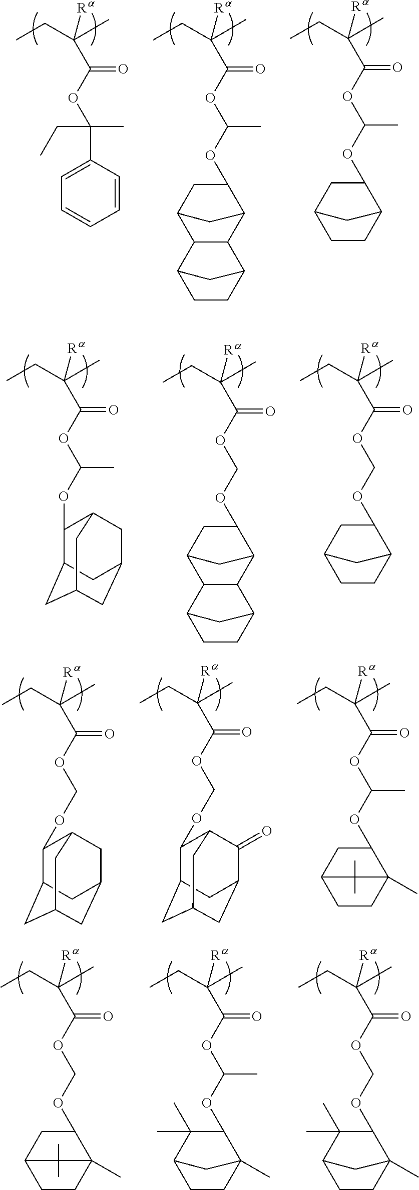

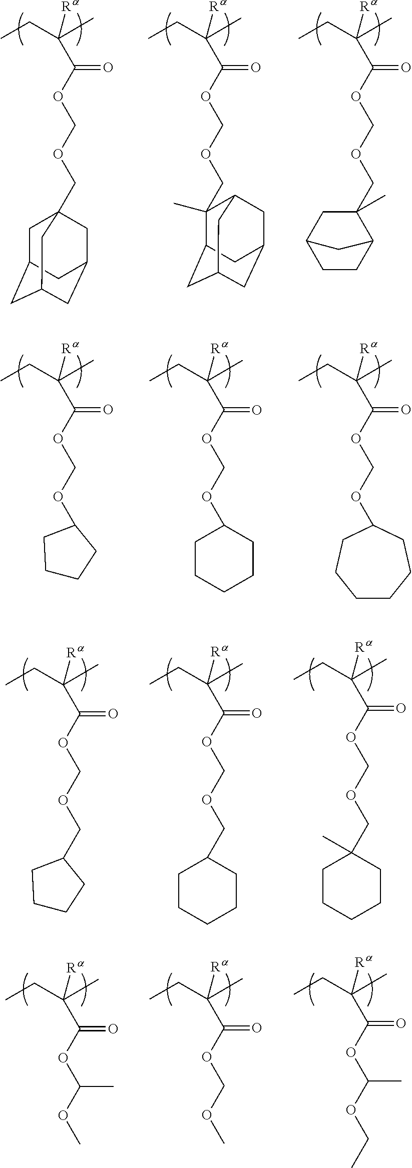

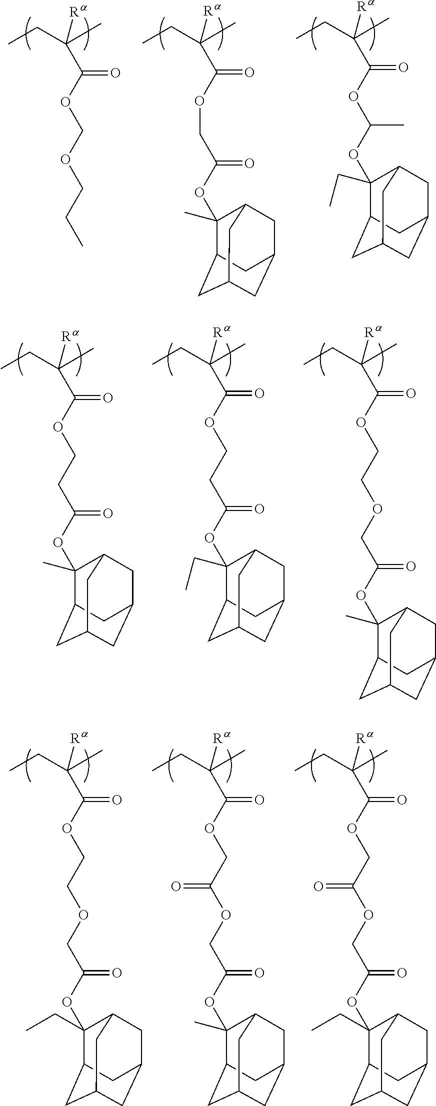

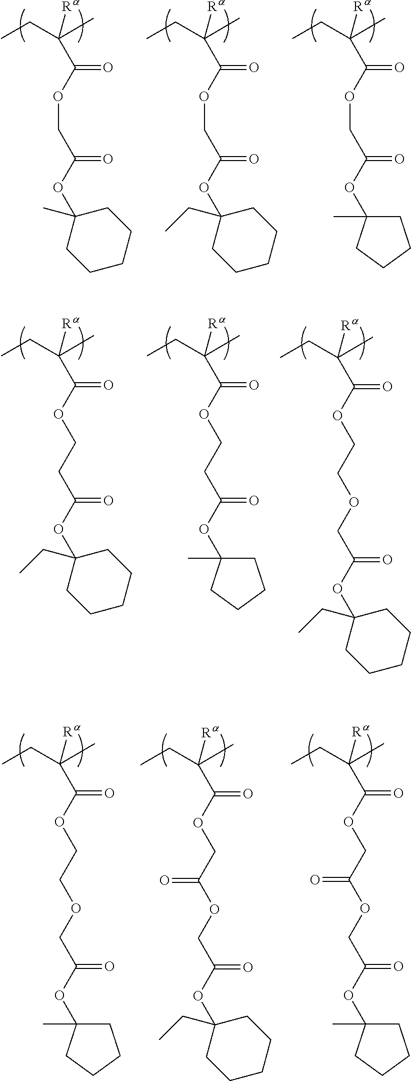

Specific examples of the constitutional unit represented by Formula (a1-2) are shown below.

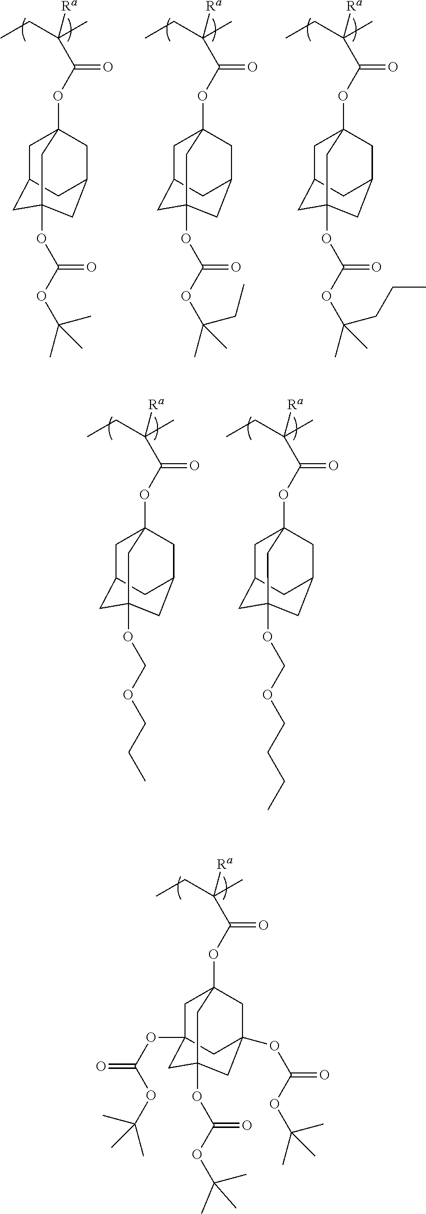

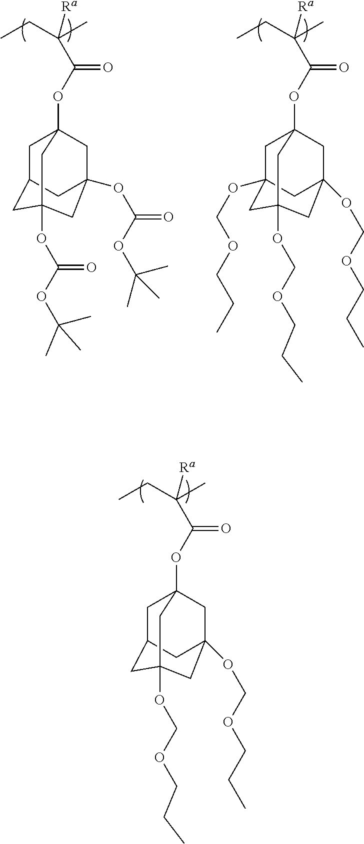

The constitutional unit (a1) included in the component (A1) may be used alone or two or more kinds thereof.

Since the lithography characteristics (the sensitivity, the shape, and the like) are easily improved using electron beams or EU V, a constitutional unit represented by Formula (a1-1) is preferable as the constitutional unit (a1).

In a case where the component (A1) has the constitutional unit (a1), the proportion of the constitutional unit (a1) in the component (A1) is preferably 30% by mole or less, more preferably in a range of 1% to 25% by mole, and still more preferably in a range of 5% to 20% by mole with respect to the total amount (100% by mole) of all constitutional units constituting the component (A1). In a case where the proportion of the constitutional unit (a1) is set to be less than or equal to the upper limits, the balance between the constitutional unit (all and other constitutional units can be achieved. Meanwhile, in a case where the proportion of the constitutional unit (a1) is set to be greater than or equal to the lower limits, the lithography characteristics such as the sensitivity, the resolution, and improvement of roughness are also improved.

### In Regard to Constitutional Unit (a2):

The component (A1) may further have a constitutional unit (a2) containing a lactone-containing cyclic group, a —SO₂-containing cyclic group, or a carbonate-containing cyclic group (here, a constitutional unit corresponding to the constitutional unit (a1) is excluded).

In a case where the component (A1) is used for forming a resist film, the lactone-containing cyclic group, the —SO₂-containing cyclic group, or the carbonate-containing cyclic group in the constitutional unit (a2) is effective for improving the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

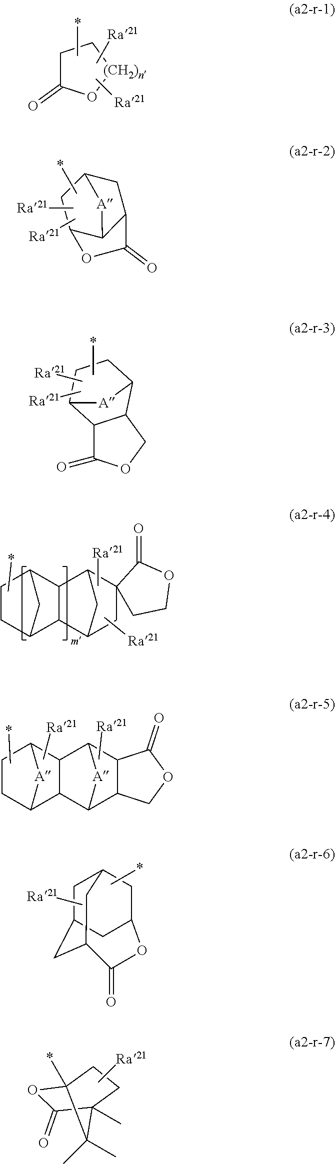

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, a carbonate-containing cyclic group, or a —SO₂-containing cyclic group; A” represents an alkylene group having 1 to 5 carbon atoms which may have an oxygen atom (—O—) or a sulfur atom (—S—), an oxygen atom, or a sulfur atom; n′ represents an integer of 0 to 2; and m′ represents 0 or 1.]

In General Formulae (a2-r-1) to (a2-r-7), it is preferable that the alkyl group as Ra′²¹ is an alkyl group having 1 to 6 carbon atom. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group exemplified as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, a lactone-containing cyclic group, a carbonate-containing cyclic group, or a —SO₂-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

The carbonate-containing cyclic group as R″ has the same definition as that for the carbonate-containing cyclic group described below. Specific examples of the carbonate-containing cyclic group include groups each represented by General Formulae (ax3-r-1) to (ax3-r-3).

The —SO₂-containing cyclic group as R″ has the same definition as that for the —SO₂-containing cyclic group described below, and specific examples thereof include a group represented by any of General Formulae (a5-r-1) to (a5-r-4).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3) and (a2-r-5), as the alkylene group having 1 to 5 carbon atoms as A”, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed between the terminals or carbon atoms of the alkylene group. Further, examples thereof include —O—CH₂—, —CH₂—O—CH₂—, —S—CH₂—, and —CH₂—S—CH₂—. A” represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

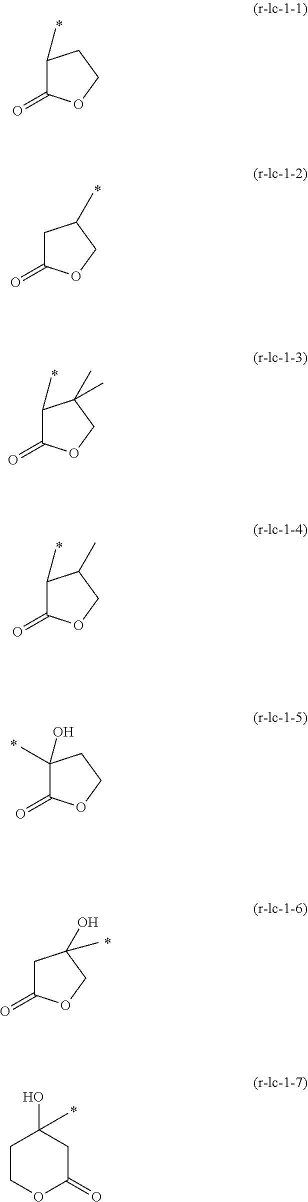

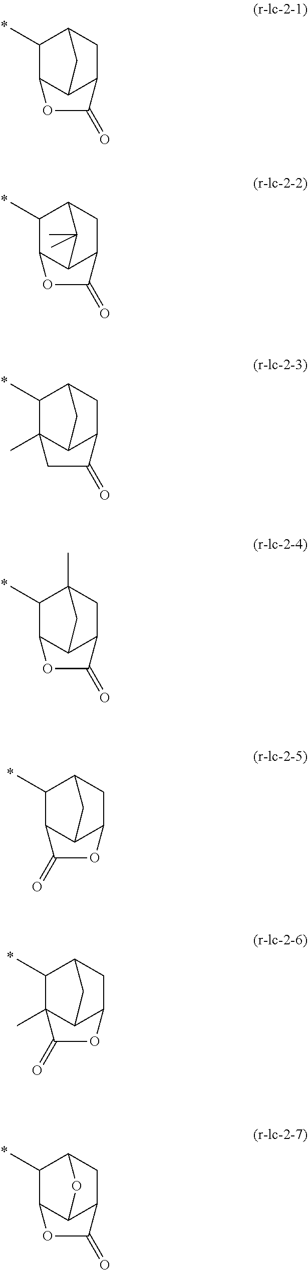

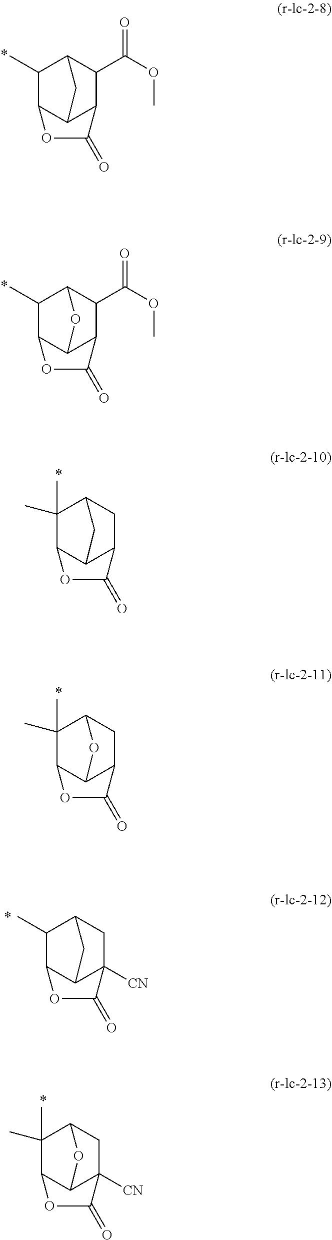

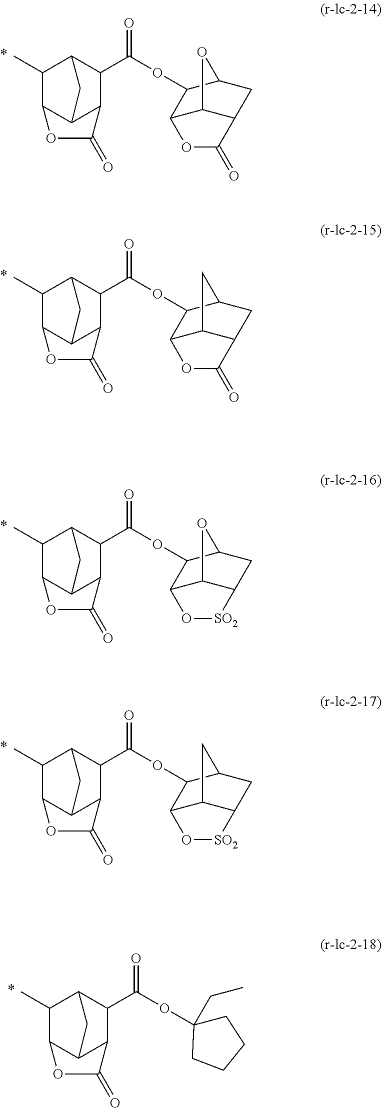

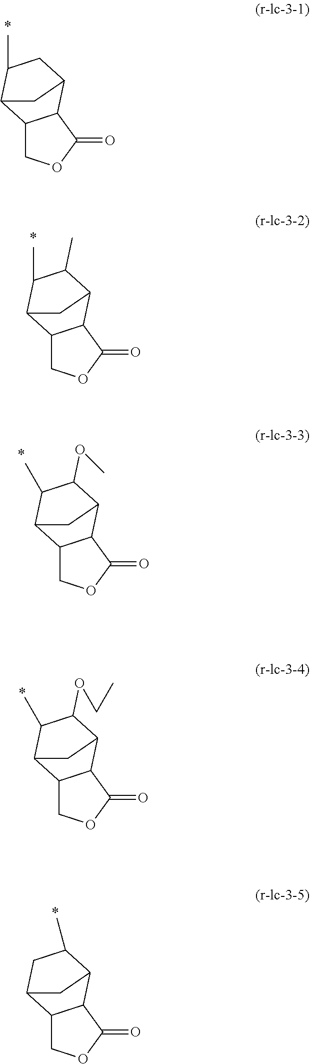

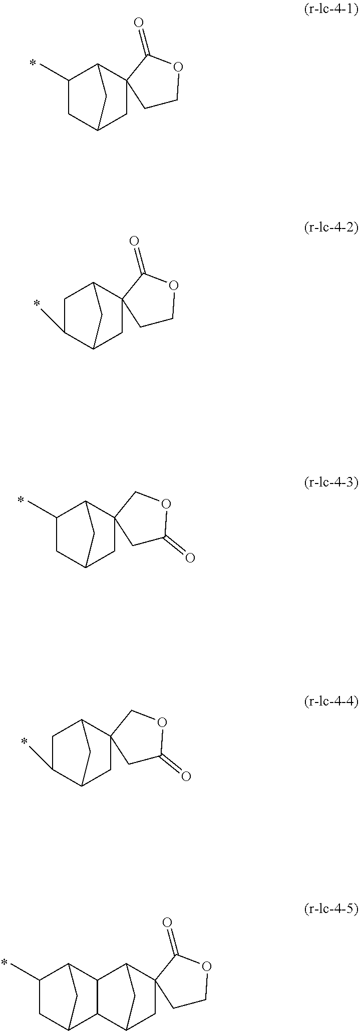

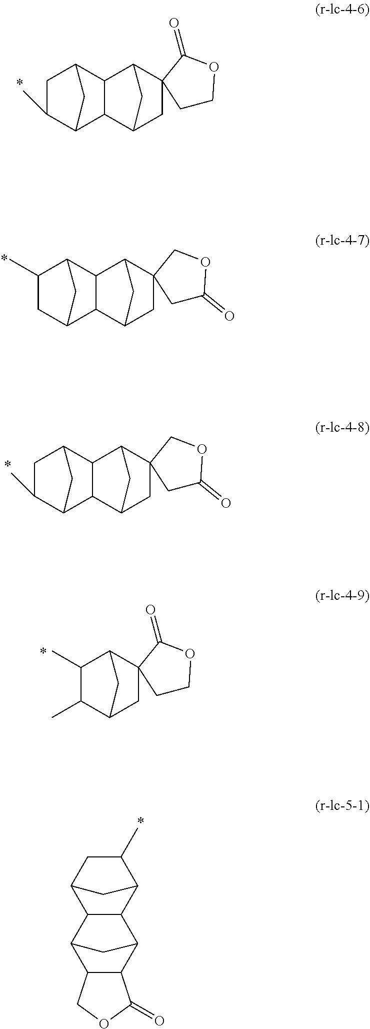

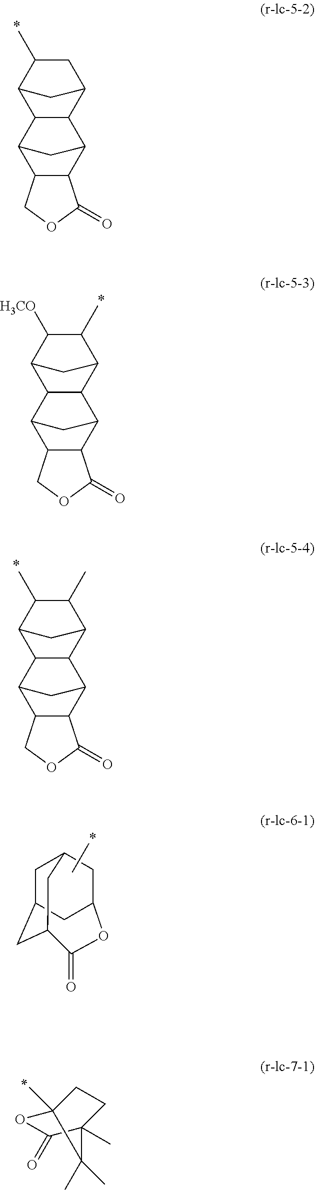

The term “—SO₂-containing cyclic group” denotes a cyclic group having a ring containing —SO₂— in the ring skeleton thereof. Specifically, the —SO₂-containing cyclic group is a cyclic group in which the sulfur atom (S) in —SO₂— forms a part of the ring skeleton of the cyclic group. In a case where the ring containing —SO₂— in the ring skeleton thereof is counted as the first ring and the group has only the ring, the group is referred to as a monocyclic group. In a case where the group further has other ring structures, the group is referred to as a polycyclic group regardless of the structures. The —SO₂-containing cyclic group may be a monocyclic group or a polycyclic group. The —SO₂-containing cyclic group is particularly preferably a cyclic group containing —O—SO₂— in the ring skeleton thereof, that is, a cyclic group containing a sultone ring in which —O—S— in —O—SO₂— forms a part of the ring skeleton thereof.

More specific examples of the —SO₂-containing cyclic group include groups each represented by General Formulae (a5-r-1) to (a5-r-4).

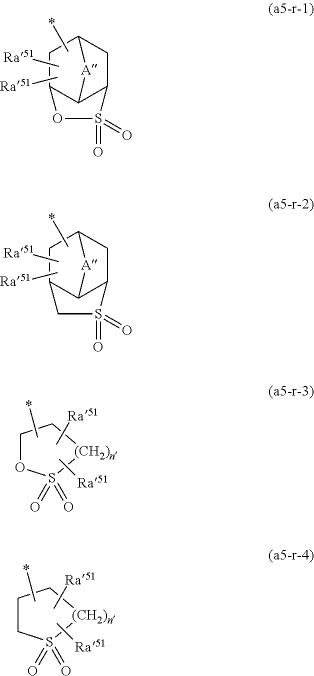

[In the formulae, Ra′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, a carbonate-containing cyclic group, or a —SO₂-containing cyclic group, A” represents an alkylene group having 1 to 5 carbon atoms which may have an oxygen atom or a sulfur atom, an oxygen atom, or a sulfur atom, and n′ represents an integer of 0 to 2.]

In General Formulae (a5-r-1) and (a5-r-2), A” has the same definition as that for A” in Formulae (a2-r-2), (a2-r-3) and (a2-r-5).

Examples of the alkyl group, the alkoxy group, the halogen atom, the halogenated alkyl group, —COOR″, —OC(═O)R″, and the hydroxyalkyl group as Ra′⁵¹ include the same groups as those for Ra′²¹ in General Formulae (a2-r-1) to (a2-r-7).

Specific examples of the groups each represented by General Formulae (a5-r-1) to (a5-r-4) are shown below. In the formulae shown below, “Ac” represents an acetyl group.

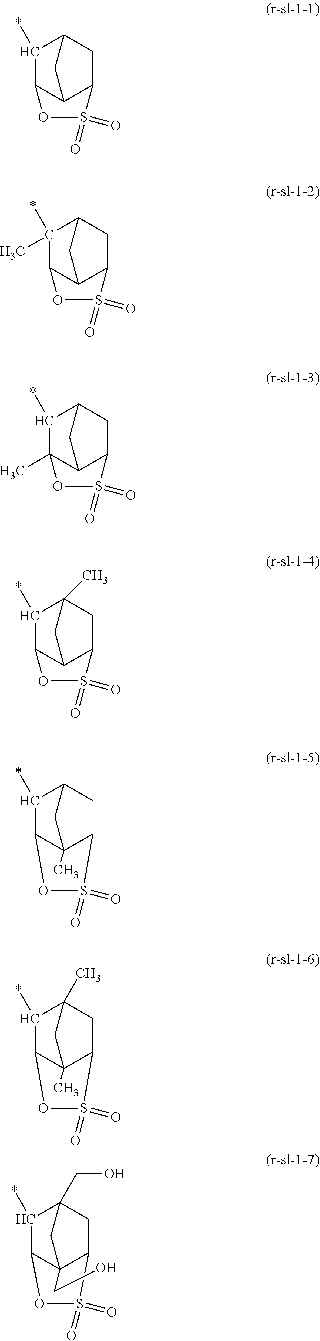

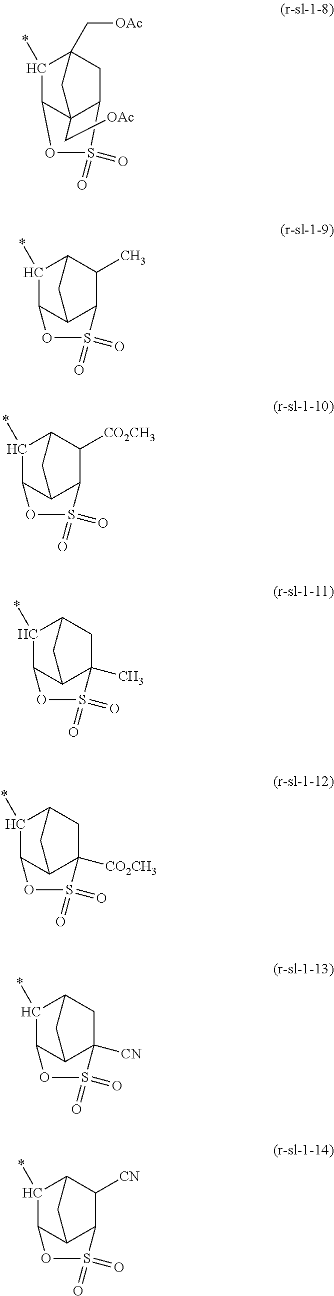

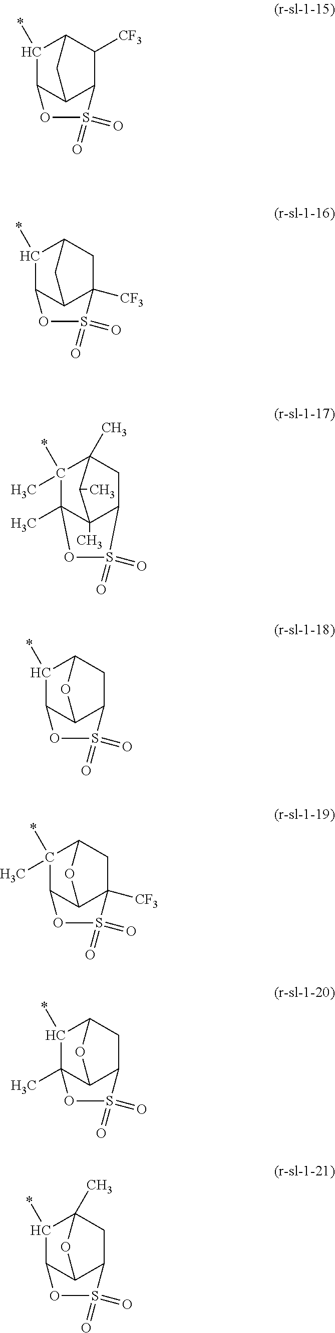

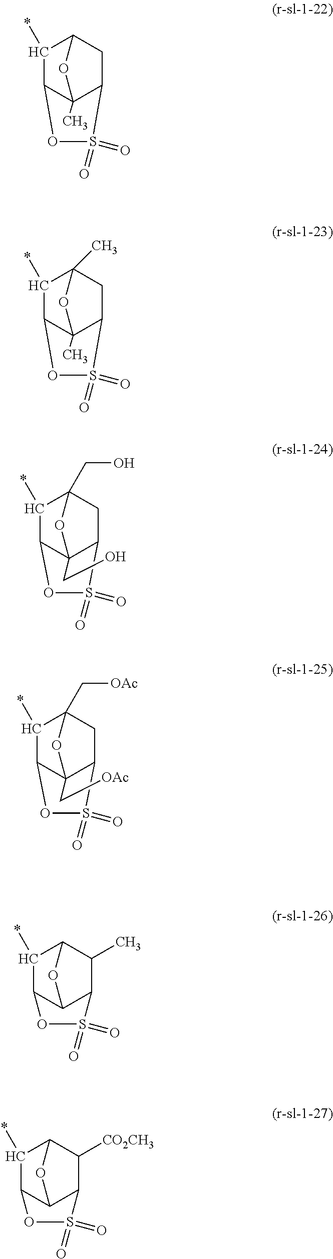

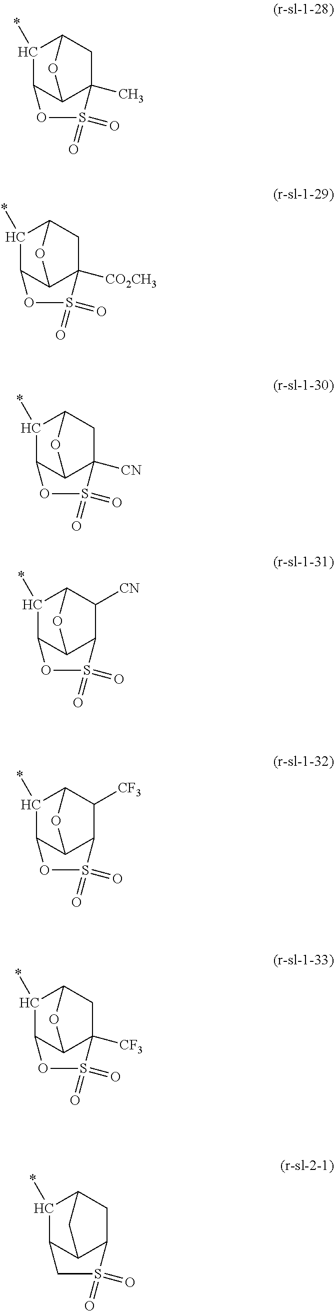

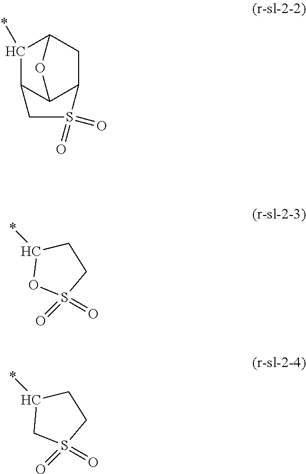

The term “carbonate-containing cyclic group” indicates a cyclic group that has a ring (a carbonate ring) containing —O—C(═O)—O— in the ring skeleton thereof. In a case where the carbonate ring is counted as the first ring and the group has only the carbonate ring, the group is referred to as a monocyclic group. Further, in a case where the group has other ring structures, the group is referred to as a polycyclic group regardless of the structures. The carbonate-containing cyclic group may be a monocyclic group or a polycyclic group.

The carbonate ring-containing cyclic group is not particularly limited, and an optional group can be used. Specific examples thereof include groups each represented by General Formulae (ax3-r-1) to (ax3-r-3) shown below.

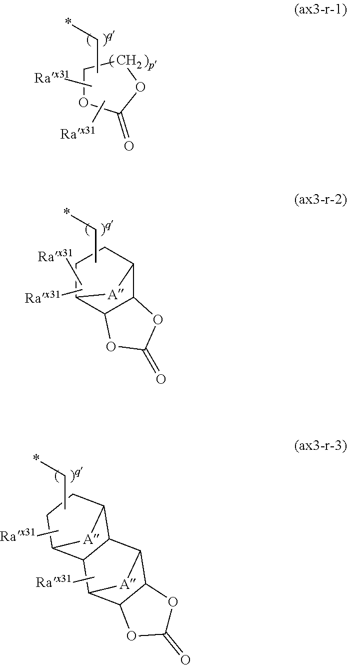

[In the formulae, Ra′^{x31}'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, a carbonate-containing cyclic group, or a —SO₂-containing cyclic group, A” represents an alkylene group having 1 to 5 carbon atoms which may have an oxygen atom or a sulfur atom, an oxygen atom, or a sulfur atom, p′ represents an integer of 0 to 3, and q′ represents 0 or 1.]

In General Formulae (ax3-r-2) and (ax3-r-3), A” has the same definition as that for A” in Formulae (a2-r-2), (a2-r-3) and (a2-r-5).

Examples of the alkyl group, the alkoxy group, the halogen atom, the halogenated alkyl group, —COOR″, —OC(═O)R″, and the hydroxyalkyl group as Ra′ include the same groups as those for Ra′²¹ in General Formulae (a2-r-1) to (a2-r-7).

Specific examples of the groups each represented by General Formulae (ax3-r-1) to (ax3-r-3) are shown below.

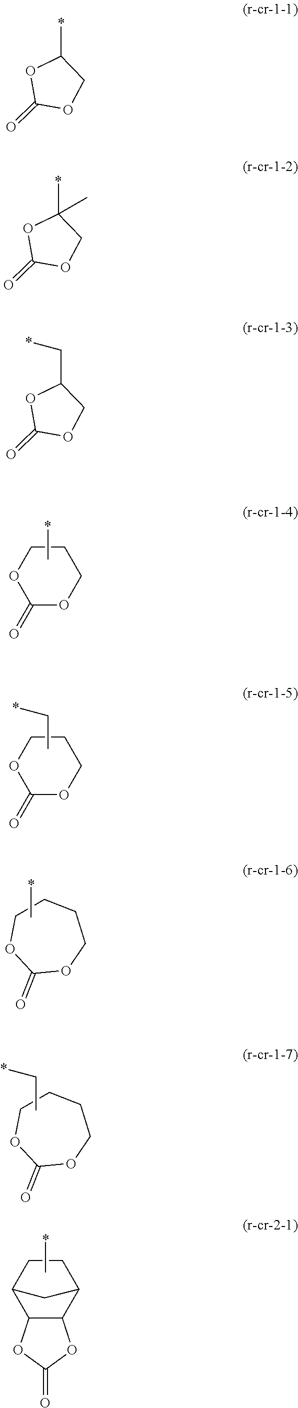

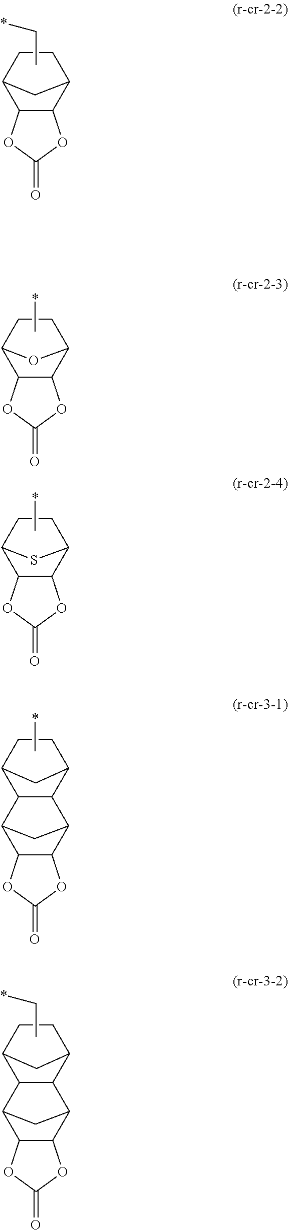

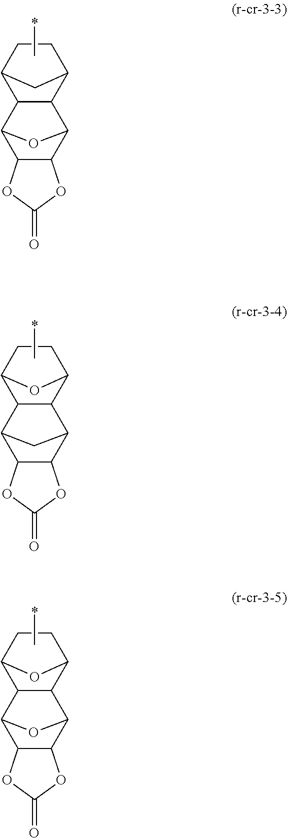

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

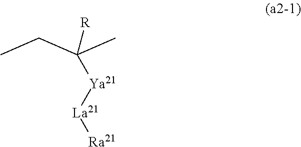

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represents —CO—. Ra²¹ represents a lactone-containing cyclic group, a carbonate-containing cyclic group, or a —SO₂-containing cyclic group.]

In Formula (a2-1), R has the same definition as that for R in Formula (a10-1). R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a hetero atom.

Examples of the divalent linking group as Ya²′ include the same groups as those for the divalent linking group as Va^{x0} in Formula (a0-1).

Among these, it is preferable that Ya²¹ represents a single bond, an ester bond [—C(═O)—O—], an ether bond (—O—), a linear or branched alkylene group, or a combination thereof.

In Formula (a2-1), Ra²¹ represents a lactone-containing cyclic group, a —SO₂-containing cyclic group, or a carbonate-containing cyclic group.

Suitable examples of the lactone-containing cyclic group, the —SO₂-containing cyclic group, and the carbonate-containing cyclic group as Ra²¹ include groups each represented by General Formulae (a2-r-1) to (a2-r-7), groups each represented by General Formulae (a5-r-1) to (a5-r-4), and groups each represented by General Formulae (ax3-r-1) to (ax3-r-3).

Among the examples, the lactone-containing cyclic group or the —SO₂-containing cyclic group is preferable, a group represented by any of General Formulae (a2-r-1), (a2-r-2), (a2-r-6), and (a5-r-1) is more preferable, and a group represented by any of General Formulae (a2-r-2) and (a5-r-1) is still more preferable. Specifically, a group represented by any of Chemical Formulae (r-1c-1-1) to (r-1c-1-7), (r-1c-2-1) to (r-1c-2-18), (r-1c-6-1), (r-s1-1-1), and (r-s1-1-18) is preferable, a group represented by any of Chemical Formulae (r-1c-2-1) to (r-1c-2-18) and (r-s1-1-1) is more preferable, and a group represented by any of Chemical Formulae (r-1c-2-1), (r-1c-2-12), and (r-s1-1-1) is still more preferable.

The constitutional unit (a2) included in the component (A1) may be used alone or two or more kinds thereof.

In a case where the component (A1) has the constitutional unit (a2), the proportion of the constitutional unit (a2) is preferably 30% by mole or less, more preferably in a range of 1% to 25% by mole, and still more preferably in a range of 5% to 20% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### In Regard to Constitutional Unit (a3):

The component (A1) may further have a constitutional unit (a3) (however, a constitutional unit corresponding to the constitutional unit (a1) or the constitutional unit (a2) is excluded) containing a polar group-containing aliphatic hydrocarbon group. In a case where the component (A1) has the constitutional unit (a3), the hydrophilicity of the component (A) is increased, which contributes to improvement of the resolution. Further, the acid diffusion length can be appropriately adjusted.

Examples of the polar group include a hydroxyl group, a cyano group, a carboxy group, or a hydroxyalkyl group in which some hydrogen atoms in the alkyl group have been substituted with fluorine atoms. Among these, a hydroxyl group is particularly preferable.

Examples of the aliphatic hydrocarbon group include a linear or branched hydrocarbon group (preferably an alkylene group) having 1 to 10 carbon atoms and a cyclic aliphatic hydrocarbon group (cyclic group). The cyclic group may be a monocyclic group or a polycyclic group. For example, the cyclic group can be appropriately selected from the plurality of groups that have been proposed in the resins for resist compositions for ArF excimer lasers.

In a case where the cyclic group is a monocyclic group, the number of carbon atoms is more preferably in a range of 3 to 10. Among the examples, constitutional units derived from acrylic acid ester that include an aliphatic monocyclic group containing a hydroxyl group, a cyano group, a carboxy group, or a hydroxyalkyl group in which some hydrogen atoms in the alkyl group have been substituted with fluorine atoms are more preferable. Examples of the monocyclic group include groups obtained by removing two or more hydrogen atoms from a monocycloalkane. Specific examples include groups obtained by removing two or more hydrogen atoms from monocycloalkanes such as cyclopentane, cyclohexane, and cyclooctane. Among these monocyclic groups, a group obtained by removing two or more hydrogen atoms from cyclopentane and a group obtained by removing two or more hydrogen atoms from cyclohexane are industrially preferable.

In a case where the cyclic group is a polycyclic group, the number of carbon atoms in the polycyclic group is more preferably in a range of 7 to 30. Among the examples, constitutional units derived from acrylic acid ester that include an aliphatic polycyclic group containing a hydroxyl group, a cyano group, a carboxy group, or a hydroxyalkyl group in which some hydrogen atoms in the alkyl group have been substituted with fluorine atoms are more preferable. Examples of the polycyclic group include groups in which two or more hydrogen atoms have been removed from bicycloalkane, tricycloalkane, tetracycloalkane, or the like. Specific examples thereof include groups in which two or more hydrogen atoms have been removed from a polycycloalkane such as adamantane, norbornane, isobornane, tricyclodecane, or tetracyclododecane. Among these polycyclic groups, a group in which two or more hydrogen atoms have been removed from adamantane, a group in which two or more hydrogen atoms have been removed from norbornane, or a group in which two or more hydrogen atoms have been removed from tetracyclododecane is industrially preferable.

The constitutional unit (a3) is not particularly limited as long as the constitutional unit contains a polar group-containing aliphatic hydrocarbon group, and an optional constitutional unit can be used.

As the constitutional unit (a3), a constitutional unit derived from acrylic acid ester in which the hydrogen atom bonded to the carbon atom at the α-position may be substituted with a substituent, which is a constitutional unit containing a polar group-containing aliphatic hydrocarbon group is preferable.

In a case where the hydrocarbon group in the polar group-containing aliphatic hydrocarbon group is a linear or branched hydrocarbon group having I to 10 carbon atoms, a constitutional unit derived from hydroxyethyl ester of acrylic acid is preferable as the constitutional unit (a3).

Further, in a case where the hydrocarbon group in the polar group-containing aliphatic hydrocarbon group is a polycyclic group, a constitutional unit represented by Formula (a3-1), a constitutional unit represented by Formula (a3-2), or a constitutional unit represented by Formula (a3-3) is preferable as the constitutional unit (a3). Further, in a case where the hydrocarbon group in the polar group-containing aliphatic hydrocarbon group is a monocyclic group, a constitutional unit represented by Formula (a3-4) is preferable as the constitutional unit (a3).

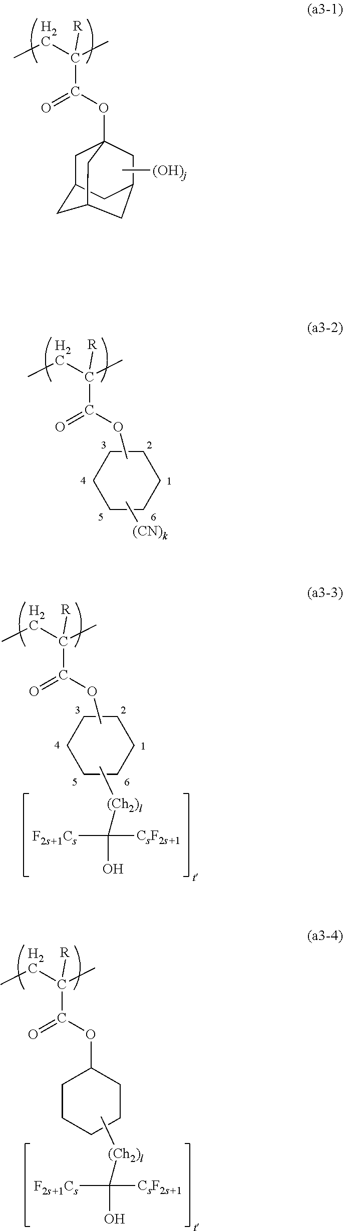

[In the formulae, R has the same definition as described above, j represents an integer of 1 to 3, k represents an integer of I to 3, t′ represents an integer of 1 to 3, 1 represents an integer of 0 to 5, and s represents an integer of 1 to 3.]

In Formula (a3-l), j represents preferably 1 or 2 and more preferably 1. In a case where j represents 2, it is preferable that the hydroxyl groups are bonded to the 3- and 5-positions of the adamantyl group. In a case where j represents 1, it is preferable that the hydroxyl group is bonded to the 3-position of the adamantyl group. It is preferable that j represents 1, and it is particularly preferable that the hydroxyl group is bonded to the 3-position of the adamantyl group.

In Formula (a3-2), it is preferable that k represents 1. It is preferable that the cyano group is bonded to the 5- or 6-position of the norbornyl group.

In Formula (a3-3), it is preferable that t′ represents 1. It is preferable that I represents 1. It is preferable that s represents 1. Further, it is preferable that a 2-norbornyl group or 3-norbornyl group is bonded to the terminal of the carboxy group of the acrylic acid. It is preferable that the fluorinated alkyl alcohol is bonded to the 5- or 6-position of the norbornyl group.

In Formula (a3-4), it is preferable that t′ represents 1 or 2. It is preferable that I represents 0 or 1. It is preferable that s represents 1. It is preferable that the fluorinated alkyl alcohol is bonded to the 3- or 5-position of the cyclohexyl group.

The constitutional unit (a3) included in the component (A1) may be used alone or two or more kinds thereof.

In a case where the component (A1) has the constitutional unit (a3), the proportion of the constitutional unit (a3) is preferably in a range of 1% to 30% by mole, more preferably in a range of 2% to 25% by mole, and still more preferably in a range of 5% to 20% by mole with respect to the total amount (100% by mole) of all constitutional units constituting the component (A1).

In a case where the proportion of the constitutional unit (a3) is set to be greater than or equal to the lower limits of the above-described preferable ranges, the effect to be obtained by allowing the component (A1) to have the constitutional unit (a3) is sufficiently obtained by the above-described effects. Further, in a case where the proportion thereof is set to be less than or equal to the upper limits of the above-described preferable ranges, the constitutional unit (a3) and other constitutional units can be balanced, and the lithography characteristics are improved.

### In Regard to Constitutional Unit (a4):

The component (A1) may further have a constitutional unit (a4) containing an acid non-dissociable aliphatic cyclic group in addition to the constitutional unit (a0).

In a case where the component (A1) has the constitutional unit (a4), the dry etching resistance of the resist pattern to be formed is further improved. Further, the hydrophobicity of the component (A) is increased. The improvement of the hydrophobicity contributes to improvement of the resolution, the resist pattern shape, and the like particularly in a case of the solvent developing process.

The term “acid non-dissociable cyclic group” in the constitutional unit (a4) denotes a cyclic group remaining in the constitutional unit without being dissociated due to the action of an acid in a case of generation of an acid in the resist composition upon light exposure (for example, an acid is generated from the component (B) or a constitutional unit that generates an acid upon light exposure).

As the constitutional unit (a4), for example, a constitutional unit derived from acrylic acid ester containing an acid non-dissociable aliphatic cyclic group or the like is preferable. As the cyclic group, a plurality of cyclic groups which have been known in the related art as those used for resin components of resist compositions for an ArF excimer laser, a KrF excimer laser (preferably an ArF excimer laser), and the like can be used.

It is particularly preferable that the cyclic group is at least one selected from a tricyclodecyl group, an adamantyl group, a tetracyclododecyl group, an isobornyl group, and a norbornyl group from the viewpoint of the industrial availability or the like. These polycyclic groups may have a linear or branched alkyl group having 1 to 5 carbon atoms as a substituent.

Specific examples of the constitutional unit (a4) include constitutional units each represented by General Formulae (a4-1) to (a4-7).

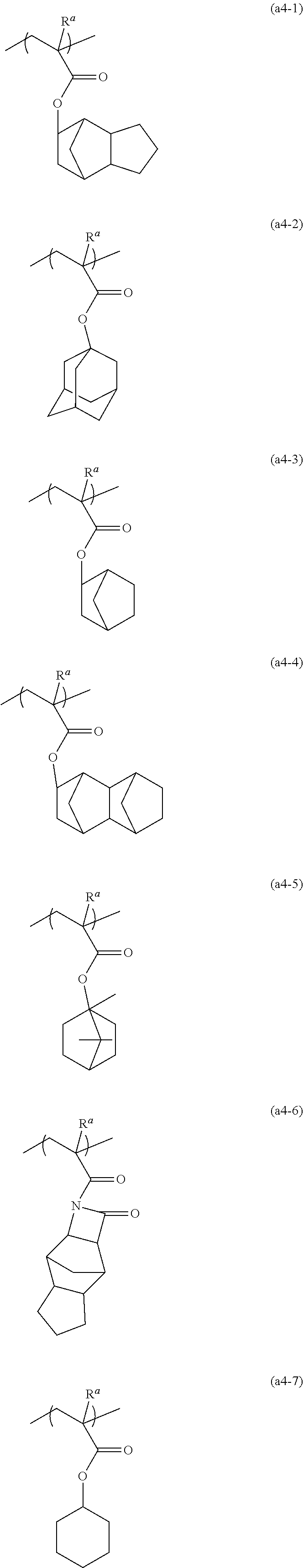

[In the formulae, R^{α} has the same definition as described above.]

The constitutional unit (a4) included in the component (A1) may be used alone or two or more kinds thereof.

In a case where the component (A1) has the constitutional unit (a4), the proportion of the constitutional unit (a4) is preferably in a range of 1% to 30% by mole and more preferably in a range of 5% to 20% by mole with respect to the total amount (100% by mole) of all constitutional units constituting the component (A1).

In a case where the proportion of the constitutional unit (a4) is set to be greater than or equal to the lower limits of the above-described preferable ranges, the effect to be obtained by allowing the component (A1) to have the constitutional unit (a4) is sufficiently obtained by the above-described effects. Further, in a case where the proportion thereof is set to be less than or equal to the upper limits of the above-described preferable ranges, the constitutional unit (a4) and other constitutional units are likely to be balanced.

### In Regard to Constitutional Unit (St):

The constitutional unit (st) is a constitutional unit derived from styrene or a styrene derivative thereof. The phrase “constitutional unit derived from styrene” means a constitutional unit that is formed by the cleavage of an ethylenic double bond of styrene. The phrase “constitutional unit derived from a styrene derivative” means a constitutional unit formed by the cleavage of an ethylenic double bond of a styrene derivative.

The term “styrene derivative” denotes a compound in which at least some hydrogen atoms of styrene are substituted with a substituent. Examples of the styrene derivative include those in which the hydrogen atom at the α-position of styrene is substituted with a substituent, those in which one or more hydrogen atoms in the benzene ring of styrene are substituted with a substituent, and those in which the hydrogen atom at the α-position of styrene and one or more hydrogen atoms of the benzene ring are substituted with a substituent.

Examples of the substituent that substitutes the hydrogen atom at the α-position of styrene include an alkyl group having 1 to 5 carbon atoms and a halogenated alkyl group having 1 to 5 carbon atoms.

As the alkyl group having 1 to 5 carbon atoms, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

As the substituent that substitutes the hydrogen atom at the α-position of styrene, an alkyl group having 1 to 5 carbon atoms or a fluorinated alkyl group having 1 to 5 carbon atoms is preferable, an alkyl group having 1 to 3 carbon atoms or a fluorinated alkyl group having 1 to 3 carbon atoms is more preferable, and a methyl group is still more preferable from the viewpoint of industrial availability.

Examples of substituents that substitute the hydrogen atom of the benzene ring of styrene include an alkyl group, an alkoxy group, a halogen atom, and a halogenated alkyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable. As the halogen atom as the substituent, a fluorine atom is preferable.

Examples of the halogenated alkyl group as the substituent include groups in which some or all hydrogen atoms in the above-described alkyl groups have been substituted with the above-described halogen atoms.

As the substituent that substitutes the hydrogen atom of the benzene ring of styrene, an alkyl group having 1 to 5 carbon atoms is preferable, a methyl group or an ethyl group is more preferable, and a methyl group is still more preferable.

As the constitutional unit (st), a constitutional unit derived from styrene or a constitutional unit derived from a styrene derivative in which the hydrogen atom at the α-position of styrene is substituted with an alkyl group having 1 to 5 carbon atoms or a halogenated alkyl group having 1 to 5 carbon atoms is preferable, a constitutional unit derived from styrene or a constitutional unit derived from a styrene derivative in which the hydrogen atom at the α-position of styrene is substituted with a methyl group is more preferable, and a constitutional unit derived from styrene is still more preferable.

The constitutional unit (st) included in the component (A1) may be used alone or two or more kinds thereof.

In a case where the component (A1) has the constitutional unit (st), the proportion of the constitutional unit (st) is preferably in a range of 1% to 30% by mole and more preferably in a range of 3% to 20% by mole with respect to the total amount (100% by mole) of all constitutional units constituting the component (A1).

The component (A1) contained in the resist composition according to the present embodiment may be used alone or in a combination of two or more kinds thereof.

In the resist composition according to the present embodiment, examples of the component (A1) include a polymer compound having a repeating structure of the constitutional unit (a0), and preferred examples thereof include a polymer compound having a repeating structure of the constitutional unit (a0) and the constitutional unit (a10).

Suitable examples of the component (A1) include a polymer compound having a repeating structure of the constitutional unit (a0) and a polymer compound having a repeating structure of the constitutional unit (a0) and the constitutional unit (a10). Among these, a polymer compound having a repeating structure of the constitutional unit (a0) and the constitutional unit (a10) is particularly preferable.

In the polymer compound having a repeating structure of the constitutional unit (a0) and the constitutional unit (a10), the proportion of the constitutional unit (a0) is preferably in a range of 30% to 95% by mole, more preferably in a range of 40% to 85% by mole, and still more preferably in a range of 50% to 70% by mole with respect to the total amount (100% by mole) of all constitutional units constituting the polymer compound. Further, the proportion of the constitutional unit (a10) is preferably in a range of 5% to 70% by mole, more preferably in a range of 15% to 60% by mole, and still more preferably in a range of 30% to 50% by mole with respect to the total amount (100% by mole) of all constitutional units constituting the polymer compound.

The component (A1) can be produced by dissolving a monomer from which the constitutional unit (a0) is derived and, as necessary, a monomer from which other constitutional units are derived in a polymerization solvent, adding a radical polymerization initiator such as azobisisobutyronitrile (AIBN) or dimethyl azobisisobutyrate (for example, V-601) to the solution to carry out polymerization, and performing a deprotection reaction depending on the raw material monomers.

Here, a —C(CF₃)₂—OH group may be introduced into the terminal during the polymerization using chain transfer agents such as HS—CH₂—CH₂—CH₂—C(CF₃)₂—OH in combination. A copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 2000 to 30000, still more preferably in a range of 3000 to 20000, and particularly preferably in a range of 4000 to 10000.

In a case where the Mw of the component (A1) is less than or equal to the upper limits of the above-described preferable ranges, the solubility in a resist solvent sufficient for use as a resist is likely to be obtained. In a case where the Mw of the component (A1) is greater than or equal to the lower limits of the above-described preferable ranges, the dry etching resistance and the cross-sectional shape of the resist pattern are enhanced. Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. In addition, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed due to the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion of the component (A1) is 25% by mass or greater, a resist pattern having various excellent characteristics such as the etching resistance and the effect of suppressing film reduction is likely to be formed.

In the resist composition of the present embodiment, the content of the component (A) may be adjusted according to the thickness of the resist film intended to be formed.

### <Compound (D0)>

In the resist composition according to the present embodiment, the compound (D0) is a compound represented by General Formula (d0-1) (hereinafter, also referred to as “component (D0)”).

The component (D0) acts as a quencher (acid diffusion control agent) which traps the acid generated in the resist composition upon light exposure.

The high sensitivity to radiation is likely to be achieved by using the component (D0) in a case of forming a finer resist pattern. In addition, the effect of suppressing film reduction is enhanced by a combination of the component (D0) and the component (A1).

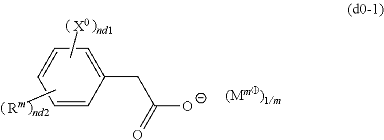

[In the formula, X⁰ represents a bromine atom or an iodine atom. R^{m} represents a hydroxy group, an alkyl group, a fluorine atom, or a chlorine atom. nd1 represents an integer of 1 to 5, and nd2 represents an integer of 0 to 4, where 1≤nd1+nd2≤5 is satisfied. Yd⁰ represents a divalent linking group or a single bond. M^{m+} represents an m-valent organic cation. m represents an integer of 1 or greater.]

### {Anion Moiety of Component (D0)}

In General Formula (d0-1), X⁰ represents a bromine atom or an iodine atom and preferably an iodine atom.

In General Formula (d0-1), R^{m} represents a hydroxy group, an alkyl group, a fluorine atom, or a chlorine atom. The alkyl group as R^{m} is preferably an alkyl group having 1 to 5 carbon atoms and more preferably a methyl group or an ethyl group.

In General Formula (d0-1), nd1 represents an integer of 1 to 5, and nd2 represents an integer of 0 to 4, where 1≤nd1+nd2≤5 is satisfied.
nd1 represents preferably an integer of 1 to 3, more preferably 2 or 3, and still more preferably 3 from the viewpoint of radiation absorbability.
nd2 represents preferably an integer of 0 to 3, more preferably 0 or 1, and still more preferably 0.

In General Formula (d0-1), Yd⁰ represents a divalent linking group or a single bond. Suitable examples of the divalent linking group as Yd⁰ include a divalent linking group containing an oxygen atom.

In a case where Yd⁰ represents a divalent linking group containing an oxygen atom, Yd⁰ may contain an atom other than the oxygen atom. Examples of the atom other than the oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination.

Yd⁰ represents preferably a divalent linking group containing an oxygen atom or a single bond and more preferably a single bond.

Specific examples of the anion moiety of the component (D0) are shown below.

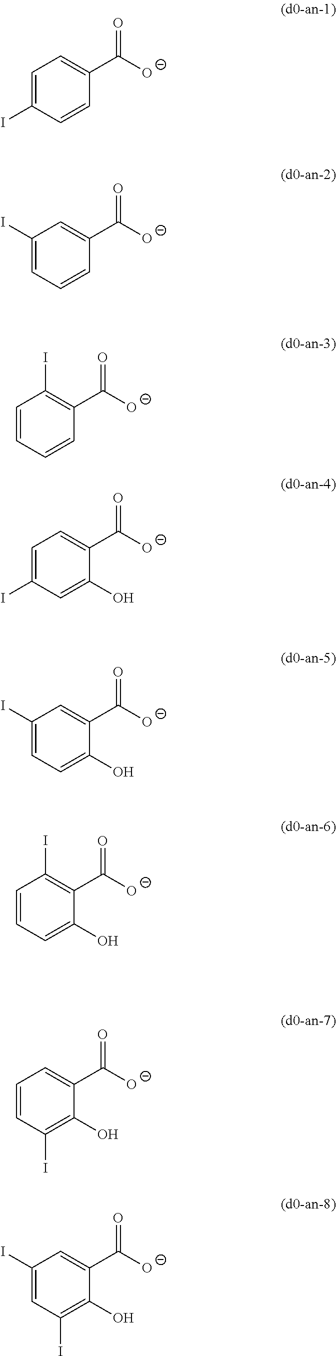

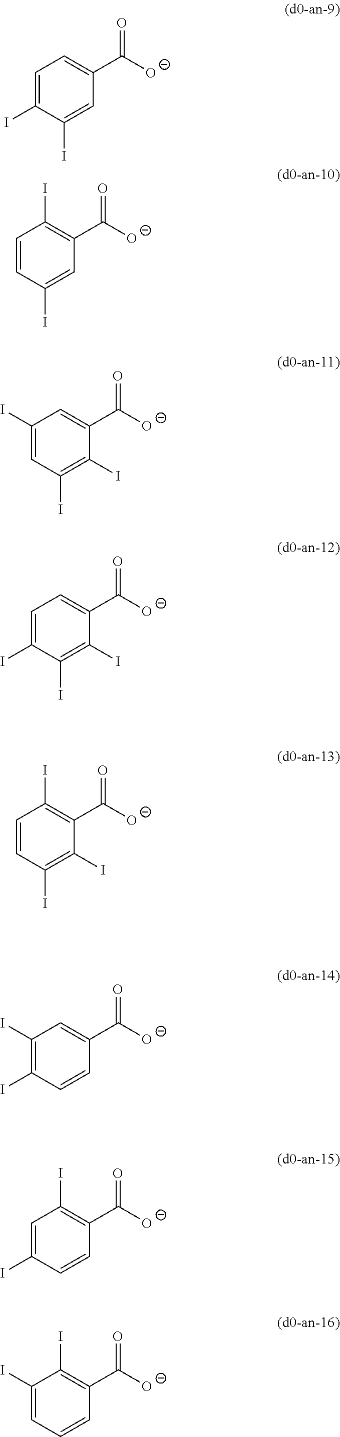

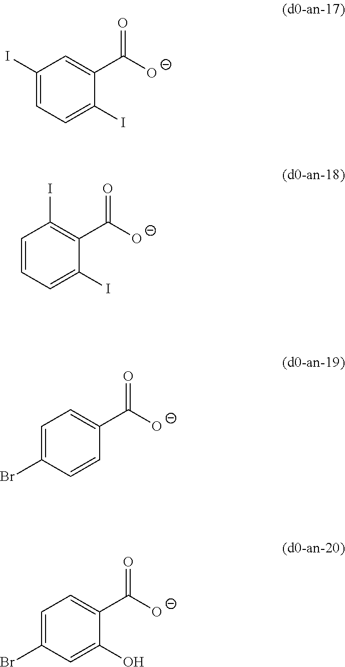 [SMILES: O=C([O-])c1cc(I)ccc1I ; O=C([O-])c1c(I)cccc1I ; O=C([O-])c1ccc(Br)cc1 ; O=C([O-])c1ccc(Br)cc1O]

### {Cation Moiety of Component (D0)}

In General Formula (d0-i), M^{m+} represents an m-valent organic cation. Among these, a sulfonium cation and an iodonium cation are preferable. m represents an integer of I or greater.

Preferred examples of the cation moiety ((M^{m+})_{l/m}) include organic cations each represented by General Formulae (ca-1) to (ca-5).

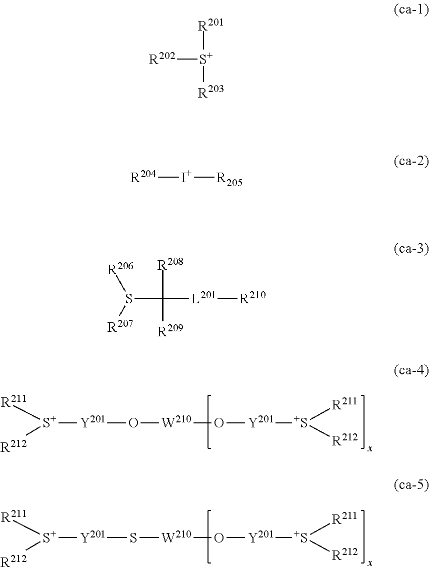

[In the formulae, R²⁰¹ to R²⁰⁷, R²¹¹, and R²¹² each independently represent an aryl group which may have a substituent, an alkyl group which may have a substituent, or an alkenyl group which may have a substituent. R²⁰¹ to R²⁰³, R²⁰⁶ and R²⁰⁷, and R²¹¹ and R²¹² may be bonded to each other to form a ring with the sulfur atom in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a SO₂-containing cyclic group which may have a substituent. L²⁰¹ represents —C(═O)— or —C(═O)—O—. Y²⁰¹'s each independently represent an arylene group, an alkylene group, or an alkenylene group. x represents I or 2. W²⁰¹ represents an (x+1)-valent linking group.]

In General Formulae (ca-1) to (ca-5), examples of the aryl group as R²⁰¹ to R²⁰⁷, R²¹¹, and R²¹² include an unsubstituted aryl group having 6 to 20 carbon atoms. Among these, a phenyl group or a naphthyl group is preferable.

The alkyl group as R²⁰¹ to R²⁰⁷, R²¹¹, and R²¹² is a chain-like or cyclic alkyl group, and the number of carbon atoms thereof is preferably in a range of 1 to 30.

It is preferable that the alkenyl group as R²⁰¹ to R²⁰⁷, R²¹¹, and R²¹² has 2 to 10 carbon atoms.

Examples of the substituent that R²⁰¹ to R²⁰⁷ and R²¹⁰ to R²¹² may have include an alkyl group, a halogen atom, a halogenated alkyl group, a carbonyl group, a cyano group, an amino group, an aryl group, and groups each represented by General Formulae (ca-r-1) to (ca-r-7).

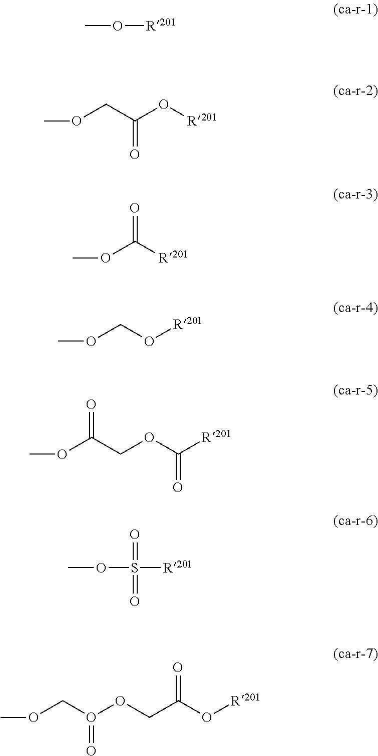

[In the formulae, R′²⁰¹'s each independently represent a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some of the carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a fused ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅-].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R′201 may have a hetero atom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and heterocyclic groups each represented by Chemical Formulae (r-hr-1) to (r-hr-16).

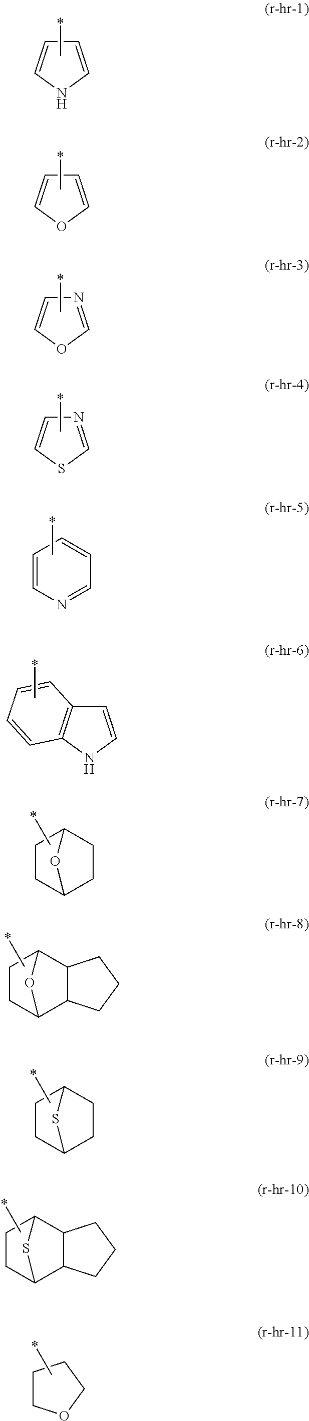

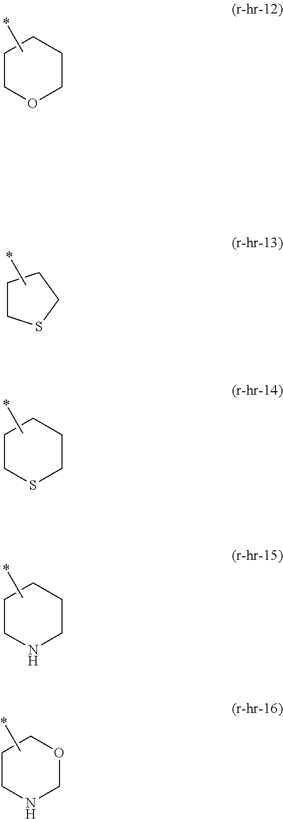

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group. Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹

Examples of the cyclic group which may have a substituent, the chain-like alkyl group which may have a substituent, and the chain-like alkenyl group which may have a substituent as R′²⁰¹ include those for the acid dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (a5-r-1) to (a5-r-4).

In General Formulae (ca-1) to (ca-5), in a case where R²⁰¹ to R²⁰³, R²⁰⁶ and R²⁰⁷, and R^{21′} and R²¹² are bonded to each other to form a ring with a sulfur atom in the formula, these groups may be bonded to each other via a hetero atom such as a sulfur atom, an oxygen atom, or a nitrogen atom, or a functional group such as a carbonyl group, —SO—, —SO₂—, —SO₃—, —COO—, —CONH— or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a SO₂-containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ has 2 to 10 carbon atoms.

As the SO₂-containing cyclic group as R²¹⁰ which may have a substituent, “—SO₂-containing polycyclic group” is preferable, and a group represented by General Formula (a5-r-1) is more preferable.

Y²⁰¹'s each independently represent an arylene group, an alkylene group, or an alkenylene group.

Examples of the arylene group as Y²⁰¹ include a group in which one hydrogen atom has been removed from the aryl group described as the aromatic hydrocarbon group represented by R′²⁰¹ in General Formulae (ca-r-1) to (ca-r-7).

Examples of the alkylene group and alkenylene group as Y^{20′} include a group in which one hydrogen atom has been removed from the chain-like alkyl group or the chain-like alkenyl group as R′²⁰¹ in General Formulae (ca-r-1) to (ca-r-7).

In Formula (ca-4), x represents 1 or 2.

W²⁰¹ represents an (x+1)-valent linking group, that is, a divalent or trivalent linking group.

As the divalent linking group represented by W²⁰¹, a divalent hydrocarbon group which may have a substituent is preferable, and examples thereof include the same divalent hydrocarbon groups which may have a substituent as those for Va^{x0} in General Formula (a0-1). The divalent linking group as W²⁰¹ may be any of linear, branched, or cyclic and is preferably cyclic. Among these, a group in which two carbonyl groups are combined with both ends of the arylene group is preferable. Examples of the arylene group include a phenylene group and a naphthylene group. Among these, a phenylene group is particularly preferable.

Examples of the trivalent linking group as W²⁰¹ include a group in which one hydrogen atom has been removed from the above-described divalent linking group as W²⁰¹ and a group obtained by bonding the divalent linking group to another divalent linking group described above. As the trivalent linking group as W²⁰¹, a group obtained by bonding two carbonyl groups to an arylene group is preferable.

Specific examples of suitable cations represented by Formula (ca-l) include cations each represented by Chemical Formulae (ca-1-1) to (ca-1-66).

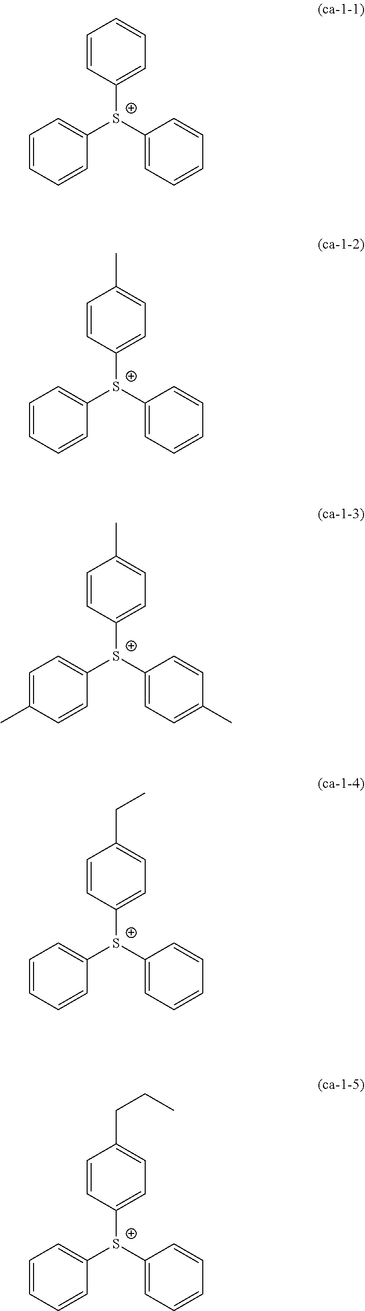

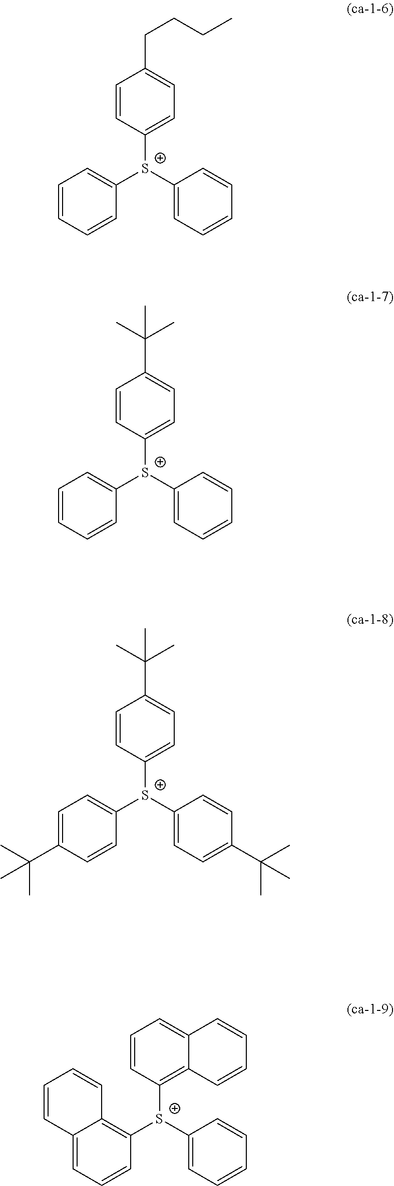 [SMILES: CCCCc1ccc([S+](c2ccccc2)c2ccccc2)cc1 ; CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1]

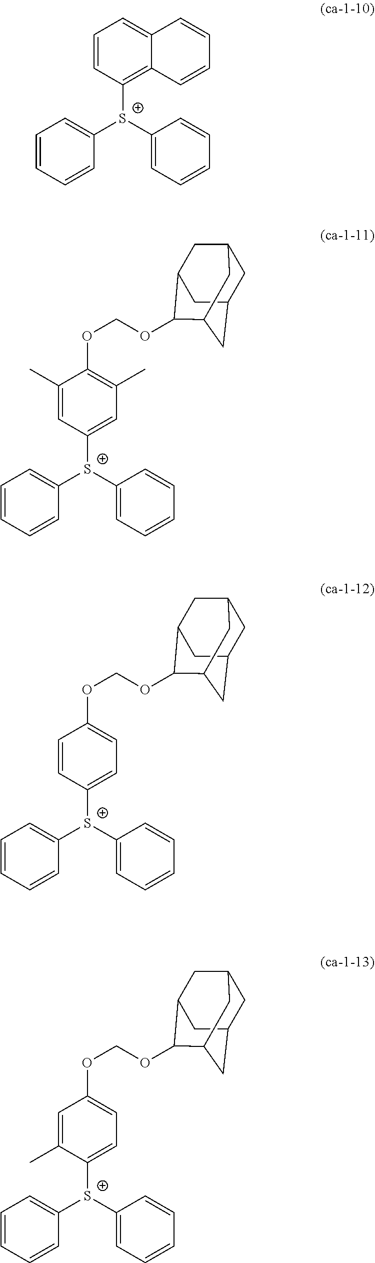 [SMILES: c1ccc([S+](c2ccccc2)c2cccc3ccccc23)cc1 ; Cc1cc([S+](c2ccccc2)c2ccccc2)cc(C)c1OCOC1C2CC3CC(C2)CC1C3 ; c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1]

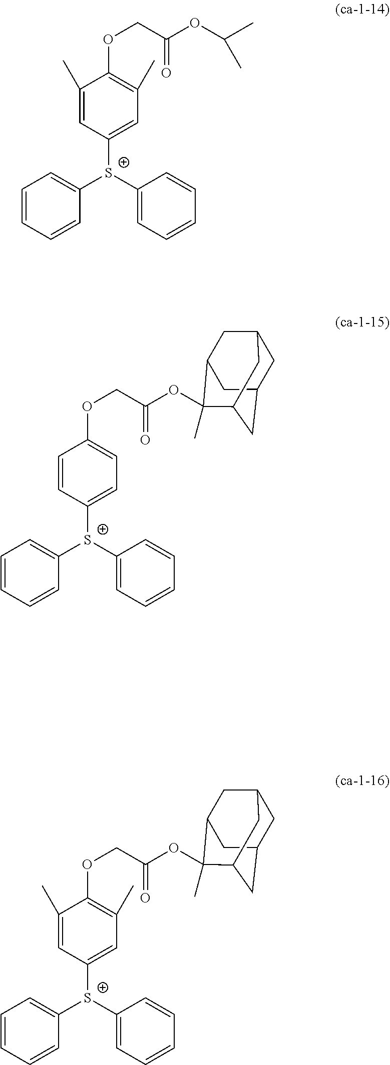 [SMILES: Cc1cc([S+](c2ccccc2)c2ccccc2)cc(C)c1OCC(=O)OC(C)C ; CC1(OC(=O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3]

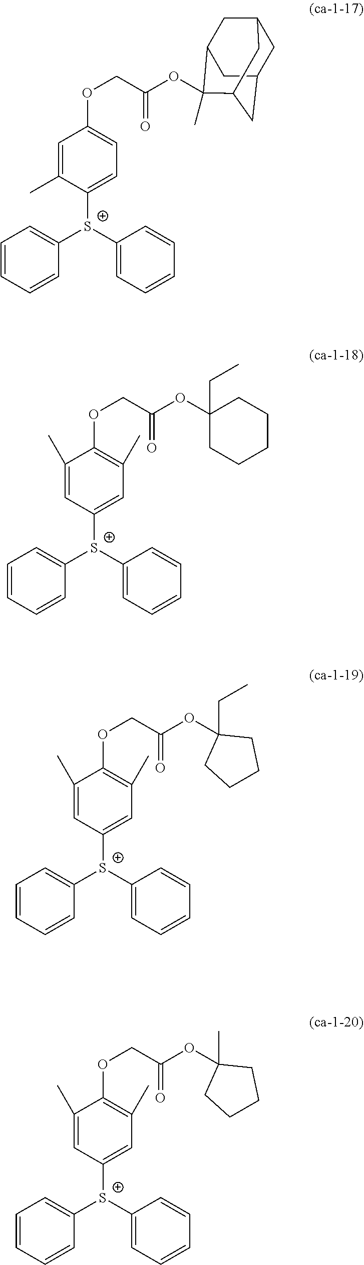 [SMILES: Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1 ; CCC1(OC(=O)COc2c(C)cc([S+](c3ccccc3)c3ccccc3)cc2C)CCCC1 ; Cc1cc([S+](c2ccccc2)c2ccccc2)cc(C)c1OCC(=O)OC1(C)CCCC1]

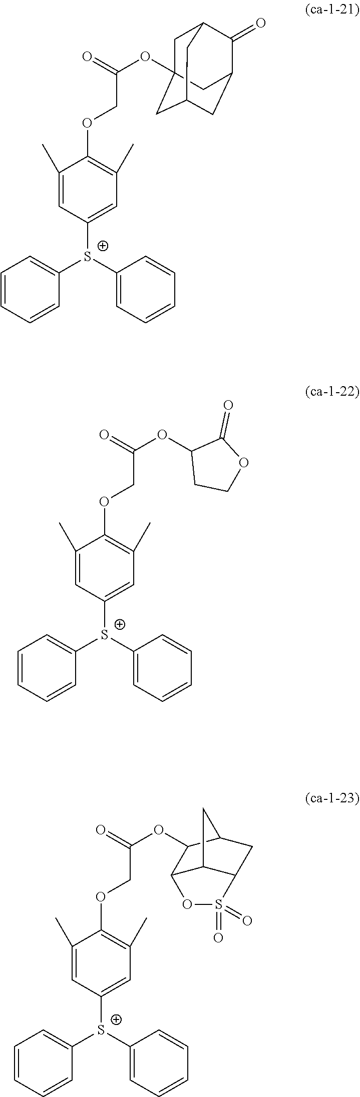 [SMILES: Cc1cc([S+](c2ccccc2)c2ccccc2)cc(C)c1OCC(=O)OC12CC3CC(C1)C(=O)C(C3)C2 ; Cc1cc([S+](c2ccccc2)c2ccccc2)cc(C)c1OCC(=O)OC1CCOC1=O ; Cc1cc([S+](c2ccccc2)c2ccccc2)cc(C)c1OCC(=O)OC1C2CC3C1OS(=O)(=O)C3C2]

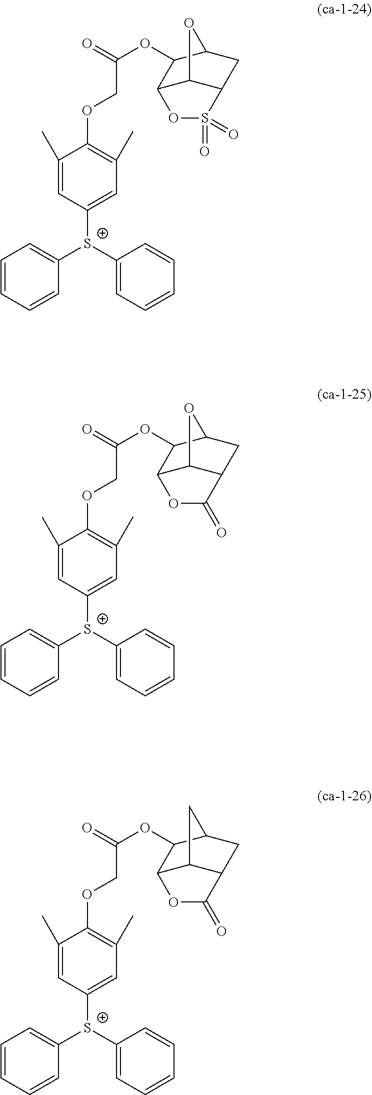 [SMILES: Cc1cc([S+](c2ccccc2)c2ccccc2)cc(C)c1OCC(=O)OC1C2CC3C(O2)C1OS3(=O)=O ; Cc1cc([S+](c2ccccc2)c2ccccc2)cc(C)c1OCC(=O)OC1C2CC3C(=O)OC1C3O2 ; Cc1cc([S+](c2ccccc2)c2ccccc2)cc(C)c1OCC(=O)OC1C2CC3C(=O)OC1C3C2]

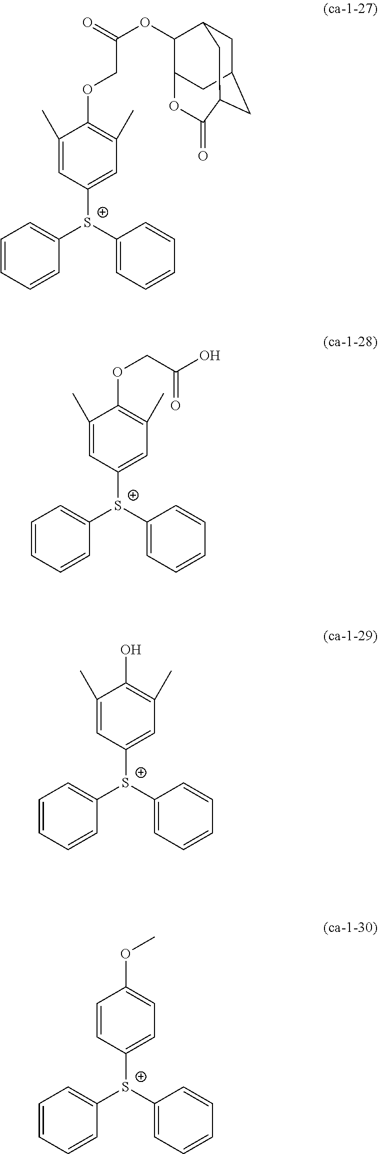 [SMILES: Cc1cc([S+](c2ccccc2)c2ccccc2)cc(C)c1OCC(=O)OC1C2CC3CC(C2)C(=O)OC1C3 ; Cc1cc([S+](c2ccccc2)c2ccccc2)cc(C)c1OCC(=O)O ; Cc1cc([S+](c2ccccc2)c2ccccc2)cc(C)c1O ; COc1ccc([S+](c2ccccc2)c2ccccc2)cc1]

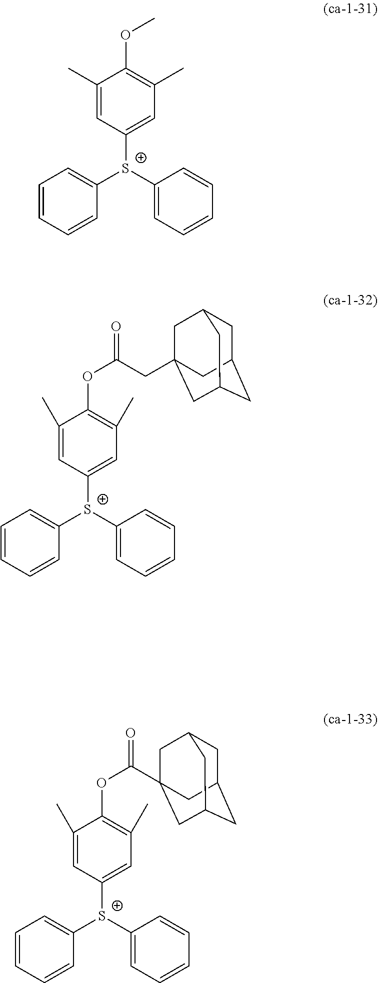 [SMILES: COc1c(C)cc([S+](c2ccccc2)c2ccccc2)cc1C ; Cc1cc([S+](c2ccccc2)c2ccccc2)cc(C)c1OC(=O)CC12CC3CC(CC(C3)C1)C2 ; Cc1cc([S+](c2ccccc2)c2ccccc2)cc(C)c1OC(=O)C12CC3CC(CC(C3)C1)C2]

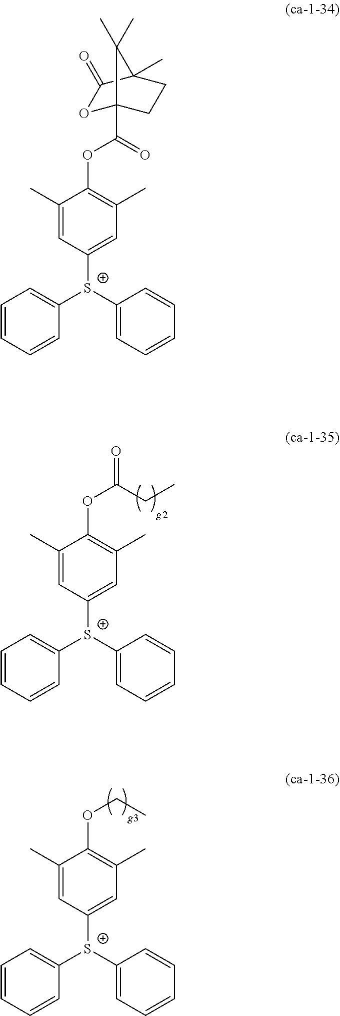

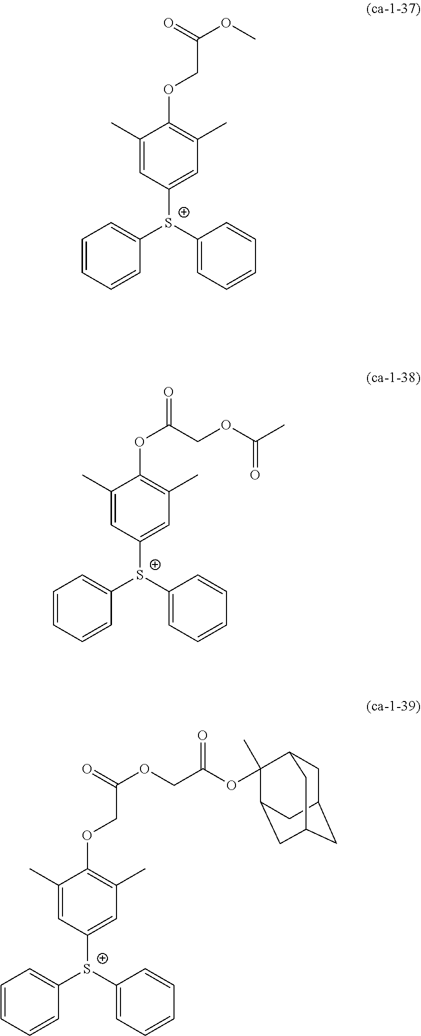 [SMILES: COC(=O)COc1c(C)cc([S+](c2ccccc2)c2ccccc2)cc1C ; CC(=O)OCC(=O)Oc1c(C)cc([S+](c2ccccc2)c2ccccc2)cc1C ; Cc1cc([S+](c2ccccc2)c2ccccc2)cc(C)c1OCC(=O)OCC(=O)OC1(C)C2CC3CC(C2)CC1C3]

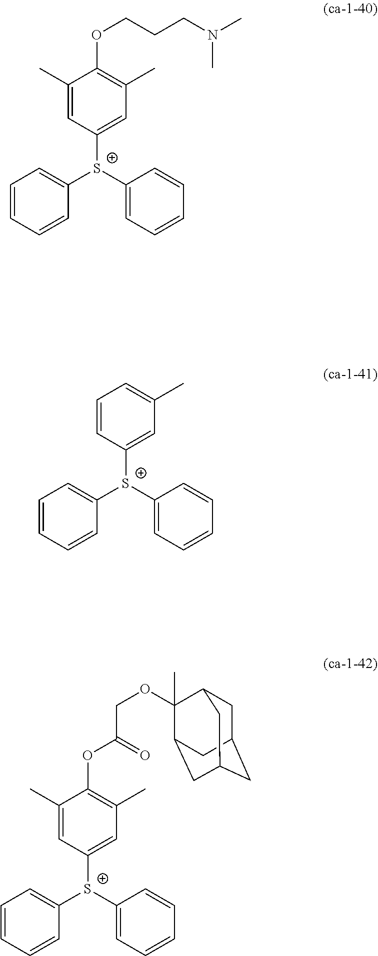 [SMILES: Cc1cc([S+](c2ccccc2)c2ccccc2)cc(C)c1OCCCN(C)C ; Cc1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3]

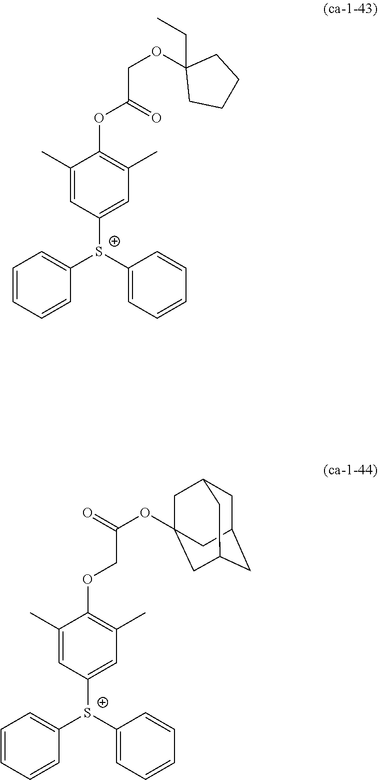 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

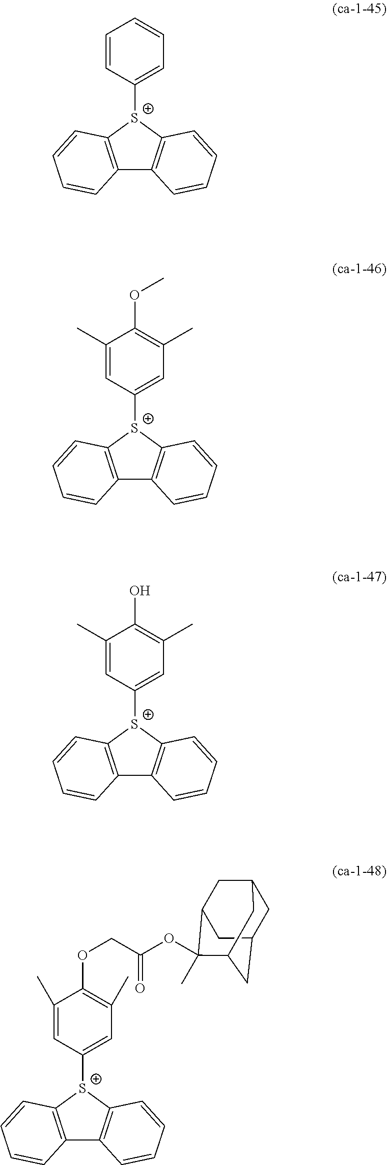 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; Cc1cc(-[s+]2c3ccccc3c3ccccc32)cc(C)c1OCC(=O)OC1(C)C2CC3CC(C2)CC1C3]

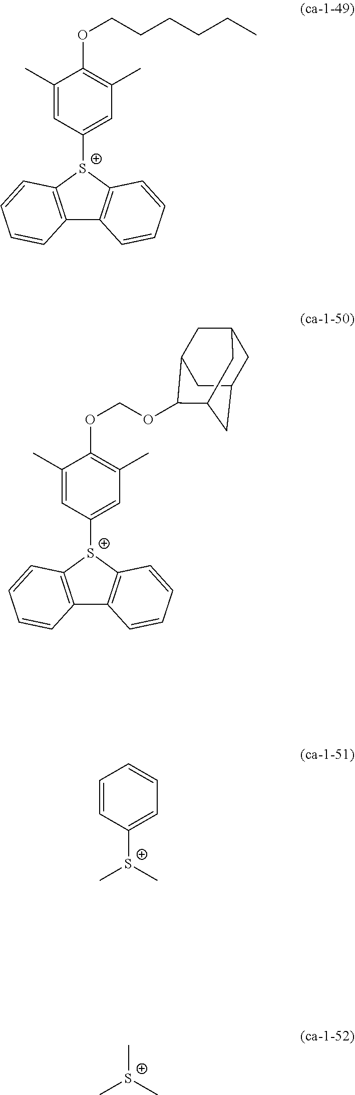

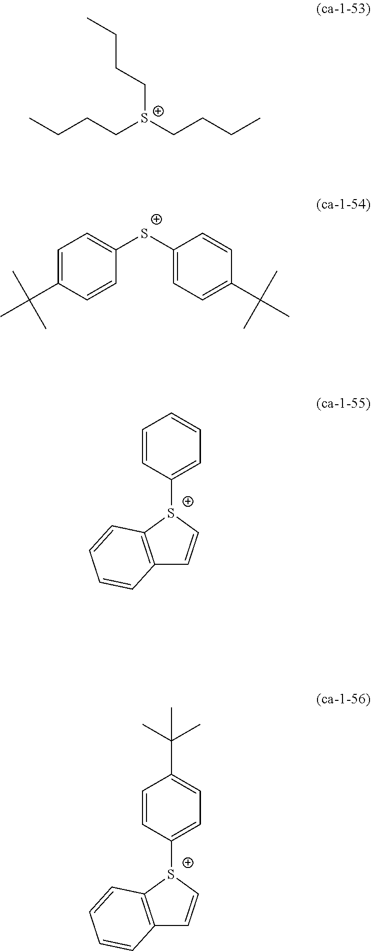

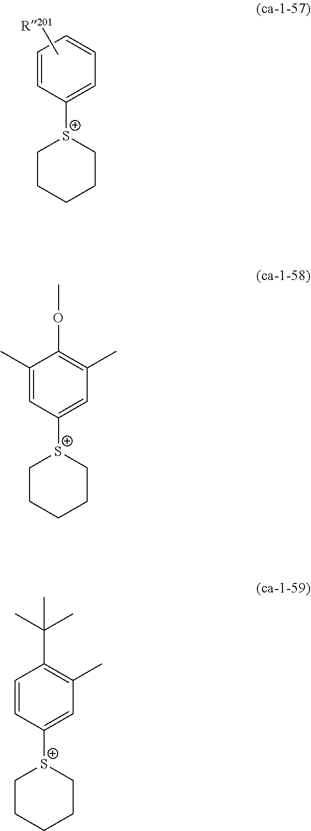

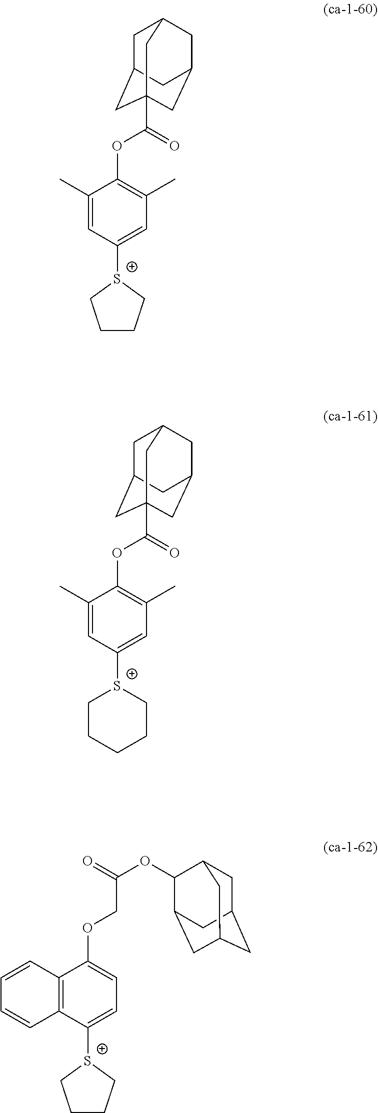 [SMILES: Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

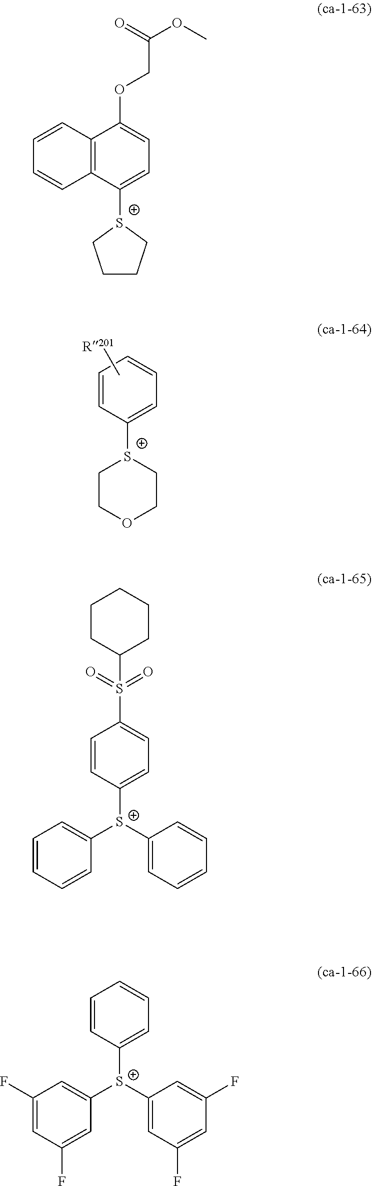

[In the formulae, R″²⁰¹ represents a hydrogen atom or a substituent, and examples of the substituent include the same groups as those for the substituents that R²01 to R²⁰⁷ and R²¹⁰ to R²¹² may have.]

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

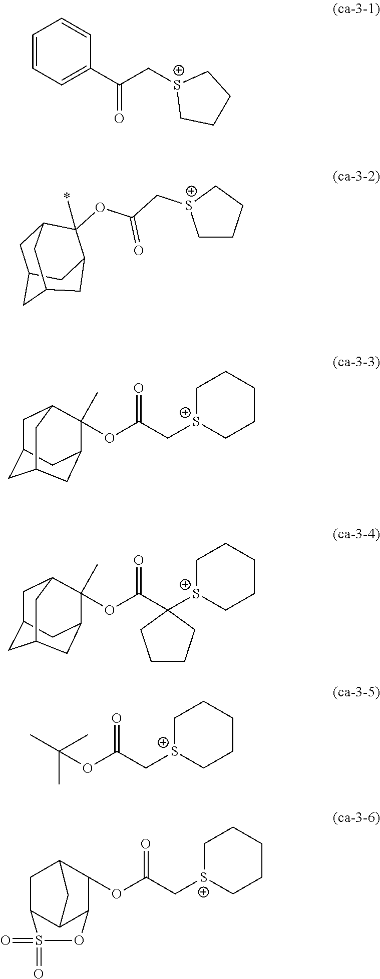 [SMILES: O=C(C[S+]1CCCC1)c1ccccc1 ; *C1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

Specific examples of suitable cations represented by Formula (ca-4) include cations each represented by Formulae (ca-4-1) and (ca-4-2).

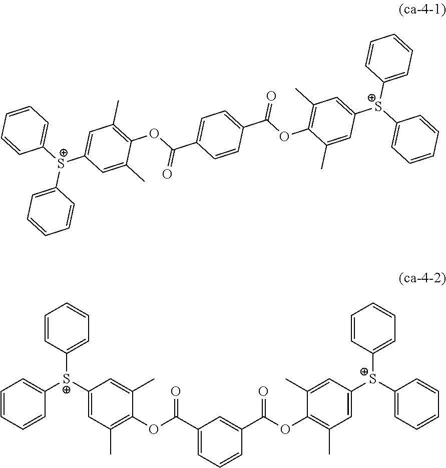 [SMILES: Cc1cc([S+](c2ccccc2)c2ccccc2)cc(C)c1OC(=O)c1ccc(C(=O)Oc2c(C)cc([S+](c3ccccc3)c3ccccc3)cc2C)cc1 ; Cc1cc([S+](c2ccccc2)c2ccccc2)cc(C)c1OC(=O)c1cccc(C(=O)Oc2c(C)cc([S+](c3ccccc3)c3ccccc3)cc2C)c1]

Specific examples of suitable cations represented by Formula (ca-5) include cations each represented by General Formulae (ca-5-1) and (ca-5-3).

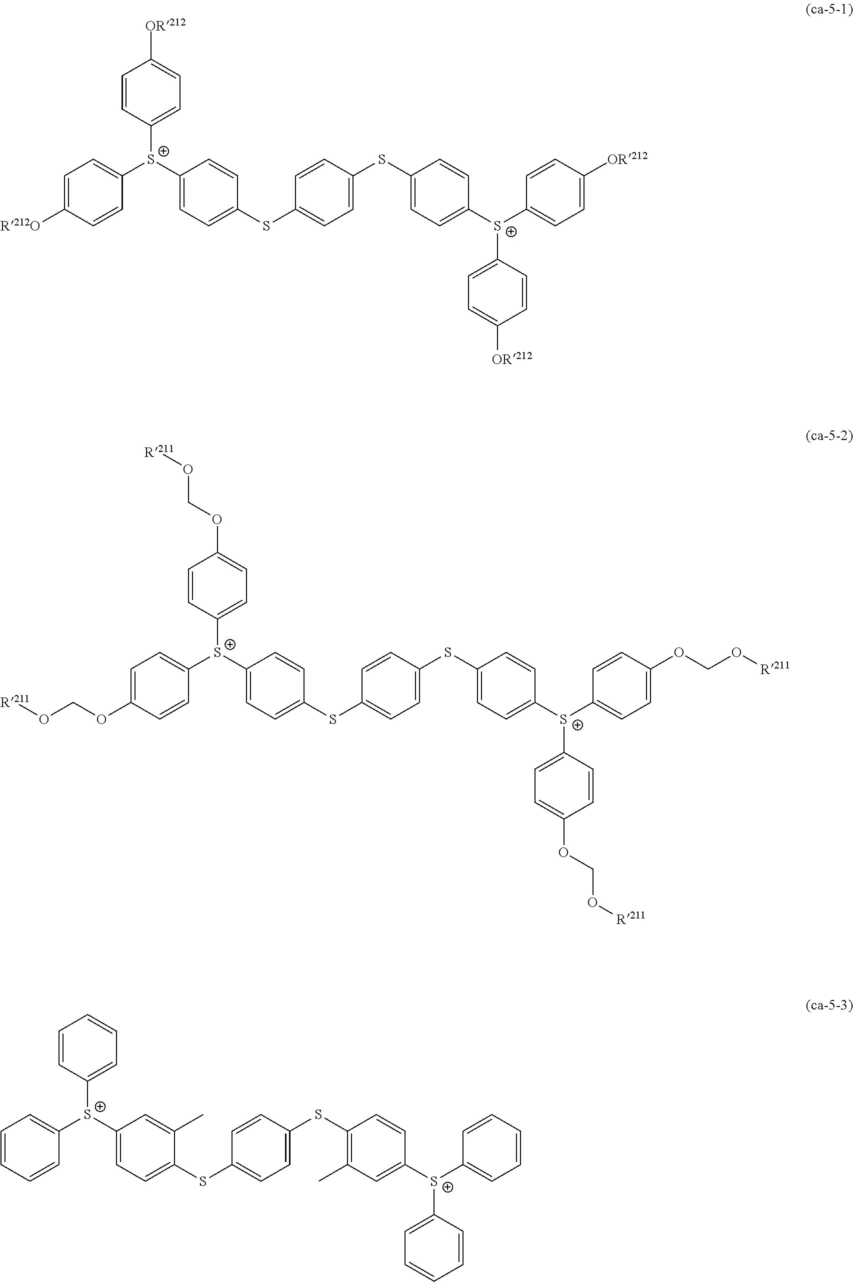

Among these, the cation moiety ((M^{m+})_{l/m}) is preferably a cation represented by General Formula (ca-1) or a cation represented by General Formula (ca-2) and more preferably a cation represented by General Formula (ca-1).

The compound (D0) is preferably a compound represented by General Formula (d0-1-1).

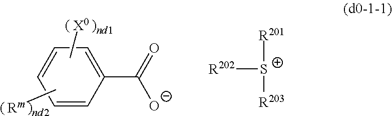

[In the formula, X⁰, R^{m}, nd1, and nd2 each have the same definition as that for X⁰, R^{m}, nd1, and nd2 in Formula (d0-1). R²⁰¹ to R²⁰³ each have the same definition as that for R²⁰¹ to R²⁰³ in Formula (ca-1).]

Specific examples of the component (D0) are shown below but are not limited thereto.

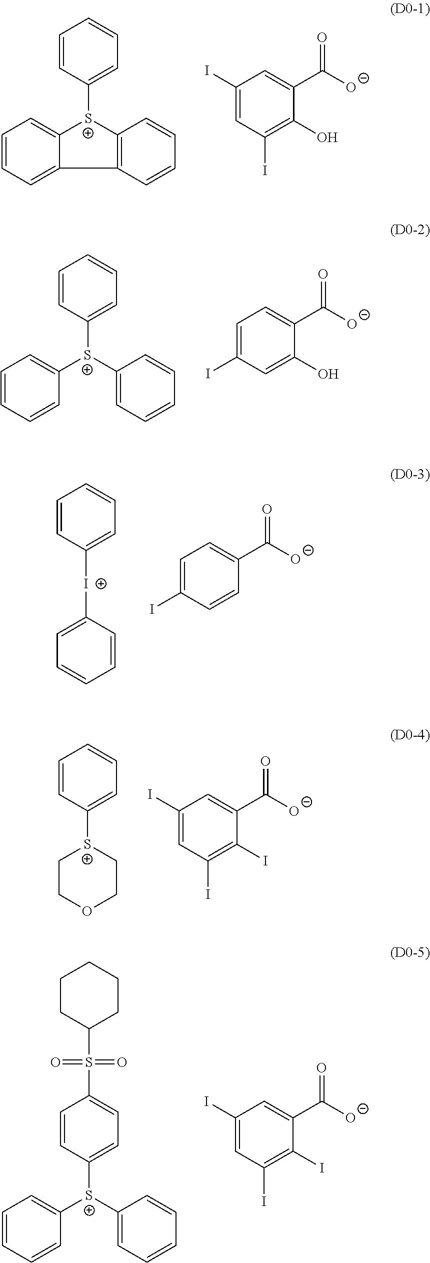

In the resist composition according to the present embodiment, the component (D0) may be used alone or may be used in a combination of two or more kinds thereof. In the resist composition according to the present embodiment, the content of the component (D0) is preferably in a range of 0.5 to 25 parts by mass, more preferably in a range of 1 to 20 parts by mass, and still more preferably in a range of 1.5 to 15 parts by mass with respect to 100 parts by mass of the component (A1).

In a case where the content of the component (D0) is greater than or equal to the lower limits of the above-described preferable ranges, the sensitivity is further enhanced and the effect of suppressing film reduction and the etching resistance are also improved in the resist pattern formation. Meanwhile, in a case where the content of the component (D0) is less than or equal to the upper limits of the above-described preferable ranges, a uniform solution is likely to be obtained in a case where each component of the resist composition is dissolved in an organic solvent, and the storage stability of the resist composition is further improved.

### <Other Components>

The resist composition according to the present embodiment may further contain other components in addition to the component (A) and the component (D0) described above. Examples of the other components include a component (B), a component (D), a component (E), a component (F), and a component (S), which are described below.

### <<Acid Generator Component (B)>>

The resist composition according to the present embodiment may further contain an acid generator component (B) that generates an acid upon light exposure (hereinafter, referred to as “component (B)”) in addition to the component (A) and the component (D0). The component (B) is not particularly limited, and those which have been proposed as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzylsulfonate-based acid generators, iminosulfonate-based acid generators, and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

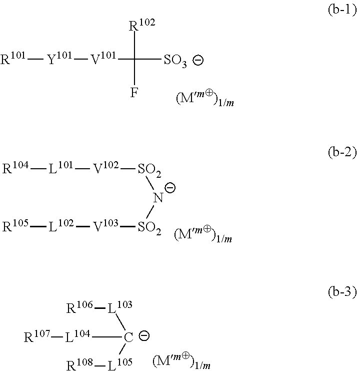

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M′^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, and an aromatic heterocyclic ring in which some of the carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a fused ring polycyclic skeleton such as a cyclic group having a steroid skeleton are preferable as the polycycloalkane.

Among these examples, as the cyclic aliphatic hydrocarbon group as R′¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰¹ may have a hetero atom such as a heterocyclic ring. Specific examples thereof include lactone-containing cyclic groups each represented by General Formulae (a2-r-1) to (a2-r-7), —SO₂-containing cyclic group each represented by General Formulae (a5-r-1) to (a5-r-4), and heterocyclic groups each represented by Chemical Formulae (r-hr-1) to (r-hr-16). In Chemical Formulae (r-hr-1) to (r-hr-16), * represents a bonding site with respect to Y¹⁰¹ in Formula (b-1).

Examples of the substituent for the cyclic group as R¹⁰¹ include an alkyl group, an alkoxy group, a halogen atom, a halogenated alkyl group, a hydroxyl group, a carbonyl group, and a nitro group. As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Examples of the halogen atom as the substituent include a fluorine atom, a chlorine atom, a bromine atom, and an iodine atom. Among these, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a fused cyclic group having a fused ring in which an aliphatic hydrocarbon ring and an aromatic ring are fused. Examples of the fused ring include those obtained by fusing one or more aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. As the fused cyclic group, a group having a fused ring in which two or three aromatic rings are fused with a bicycloalkane is preferable, and a group having a fused ring in which two or three aromatic rings are fused with bicyclo[2.2.2]octane is more preferable. Specific examples of the fused cyclic group as R¹⁰¹ include those represented by Formulae (r-br-1) and (r-br-2). In the formulae, * represents a bonding site with respect to Y¹⁰¹ in Formula (b-I).

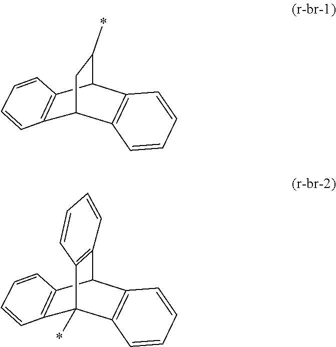 [SMILES: *C1CC2c3ccccc3C1c1ccccc12 ; *C12c3ccccc3C(c3ccccc31)c1ccccc12]

Examples of the substituent that the fused cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the fused cyclic group include those exemplified as the substituent of the cyclic group as R¹⁰¹.

Examples of the aromatic hydrocarbon group as the substituent of the fused cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Chemical Formulae (r-hr-1) to (r-hr-6).

Examples of the alicyclic hydrocarbon group as the substituent of the fused cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclodecane, or tetracyclododecane, lactone-containing cyclic groups each represented by General Formulae (a2-r-1) to (a2-r-7), —SO₂-containing cyclic groups each represented by General Formulae (a5-r-1) to (a5-r-4), and heterocyclic groups each represented by Chemical Formulae (r-hr-7) to (r-hr-16).

Chain-Like Alkyl Group which May have Substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (a5-r-1) to (a5-r-4).

In Formula (b-1), Y¹⁰¹ represents a single bond or a divalent linking group having an oxygen atom.

In a case where Y¹⁰¹ represents a divalent linking group containing an oxygen atom, Y¹⁰¹ may contain an atom other than the oxygen atom. Examples of atoms other than an oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination. Examples of such a divalent linking group having an oxygen atom include linking groups each represented by General Formulae (y-a1-1) to (y-a1-7).

Further, in General Formulae (y-a1-1) to (y-a1-7), V′¹⁰¹ in General Formulae (y-a1-1) to (y-a1-7) is bonded to R¹⁰¹ in Formula (b-1).

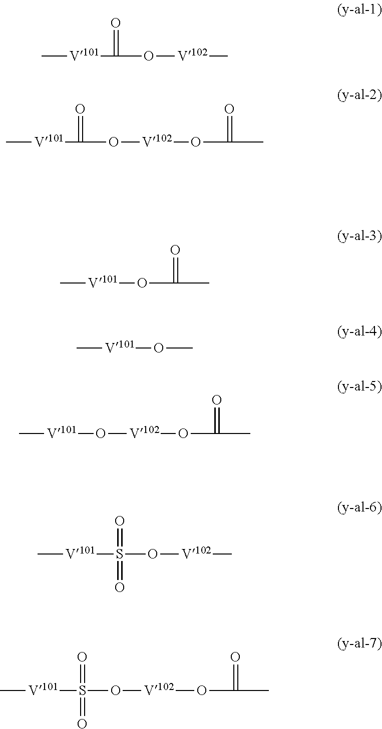

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been further removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

Y¹⁰¹ represents preferably a divalent linking group having an ester bond or a divalent linking group having an ether bond and more preferably a linking group represented by any of Formulae (y-a1-1) to (y-a1-5).

In Formula (b-1), V¹⁰¹ represents a single bond, an alkylene group, or a fluorinated alkylene group. It is preferable that the alkylene group and the fluorinated alkylene group as V¹⁰¹ have 1 to 4 carbon atoms. Examples of the fluorinated alkylene group as V¹⁰¹ include a group in which some or all hydrogen atoms in the alkylene group as V¹⁰¹ have been substituted with fluorine atoms. Among these examples, it is preferable that V¹⁰¹ represents a single bond or a fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

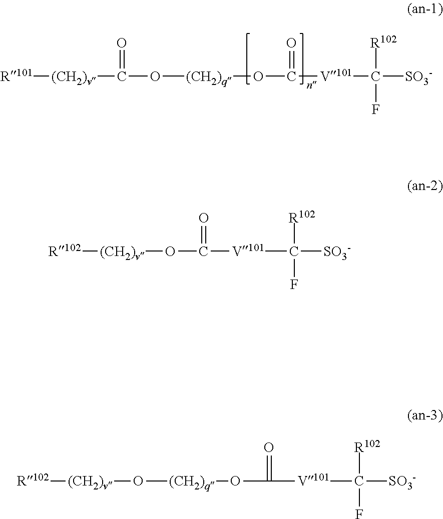

[In the formulae, R′¹⁰¹ represents an aromatic cyclic group which may have a substituent, an aliphatic cyclic group which may have a substituent, a monovalent heterocyclic group represented by any of Chemical Formulae (r-hr-1) to (r-hr-6), a fused cyclic group represented by Formula (r-br-1) or (r-br-2), or a chain-like alkyl group which may have a substituent. R″¹⁰² represents an aliphatic cyclic group which may have a substituent, a fused cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (a5-r-1) to (a5-r-4). R′¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v” independently represents an integer of 0 to 3, each q” independently represents an integer of 0 to 20, and n” represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group which may have a substituent as R″¹⁰¹ and R′¹⁰³, the same groups as those for the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

Anions in Component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms from the viewpoint that the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible from the viewpoint that the acid strength increases and the transparency to high energy light or electron beams having a wavelength of 250 nm or less is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include the same groups as those for Riot in Formula (b-1).

In Formula (b-3), L¹⁰³ to L¹⁰⁵ each independently represent a single bond, —CO—, or —SO₂—.

Among the examples, as the anion moiety of the component (B), an anion in the component (b-1) is preferable. Among the examples, an anion represented by any of General Formulae (an-1) to (an-3) is more preferable, an anion represented by General Formula (an-1) or (an-2) is still more preferable, and an anion represented by General Formula (an-1) is particularly preferable.

### {Cation Moiety}

In Formulae (b-1), (b-2), and (b-3), M^{m+} represents an m-valent onium cation. Among these, a sulfonium cation and an iodonium cation are preferable. m represents an integer of 1 or greater.

Preferred examples of the cation moiety ((M′^{m+})_{l/m}) include organic cations each represented by General Formulae (ca-1) to (ca-5). Among the examples, it is preferable that the cation moiety ((M′^{m+})_{l/m}) is a cation represented by General Formula (ca-1).

In the resist composition according to the present embodiment, the component (B) may be used alone or in combination of two or more kinds thereof.

In a case where the resist composition contains the component (B), the content of the component (B) in the resist composition is preferably less than 50 parts by mass, more preferably in a range of 1 to 40 parts by mass, still more preferably in a range of 2 to 30 parts by mass, and particularly preferably in a range of 3 to 25 parts by mass with respect to 100 parts by mass of the component (A1).

In a case where the content of the component (B) is set to be in the above-described preferable range, pattern formation can be satisfactorily performed. Further, it is preferable that each component of the resist composition is dissolved in an organic solvent from the viewpoint that a uniform solution is easily obtained and the storage stability of the resist composition is improved.

### Base Component (D)>>

The resist composition of the present embodiment may further contain, in addition to the component (A) and the component (D0), a base component that traps an acid generated upon light exposure (that is, controls diffusion of an acid).

Examples of the base component include a photodegradable base (D1) in which acid diffusion controllability is lost by decomposition upon light exposure (hereinafter, referred to as “component (D1)”) and a nitrogen-containing organic compound (D2) (hereinafter, referred to as “component (D2)”) which does not correspond to the component (D1). Among these, the photodegradable base (the component (D1)) is preferable since it is easy to enhance the roughness reducing property. Further, in a case where the component (D1) is contained, both the characteristics of increasing the sensitivity and suppressing the occurrence of coating defects are likely to be enhanced.

All of the component (D0), the component (D1), and the component (D2) act as a quencher (acid diffusion control agent) that traps an acid generated in the resist composition upon light exposure and are included in the base component (hereinafter, also referred to as “component (D)”).

In the resist composition according to the present embodiment, the proportion of the component (D0) in the total amount of the component (D) is, for example, 50% by mass or greater, preferably 70% by mass or greater, and more preferably 95% by mass or greater. The proportion of the component (D0) in the total amount of the component (D) may be 100% by mass.

In regard to component (D I)

The component (D1) is not particularly limited as long as the component is decomposed upon light exposure and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not function as a quencher at the exposed portion of the resist film, but function as a quencher at the unexposed portion of the resist film.

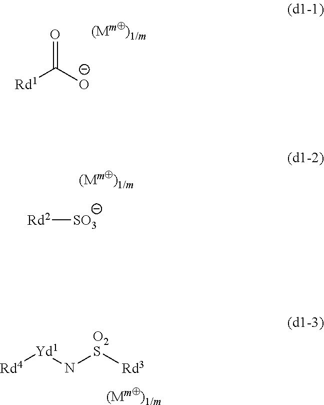

[In the formulae, Rd¹ to Rd⁴ represent a cyclic group which may have a substituent, a chain-like alkyl group which may have a substituent, or a chain-like alkenyl group which may have a substituent. Here, no fluorine atom is bonded to the carbon atom adjacent to the S atom in Rd² of Formula (d1-2). Ydⁱ represents a single bond or a divalent linking group. m represents an integer of 1 or greater. M^{m+} 's each independently represent an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹ in General Formulae (ca-r-1) to (ca-r-7).

Among these, it is preferable that Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof.

In a case where an ether bond or an ester bond is included as the substituent, the substituent may be bonded through an alkylene group, and a linking group represented by any of Formulae (y-a1-1) to (y-a1-5) is preferable as the substituent. Further, in a case where the aromatic hydrocarbon group, the aliphatic cyclic group, or the chain-like alkyl group as Rd¹ contain a linking group represented by any of General Formulae (y-a1-1) to (y-a1-7) as a substituent, V′¹⁰¹ in General Formulae (y-a1-1) to (y-a1-7) is bonded to the carbon atom constituting the aromatic hydrocarbon group, the aliphatic cyclic group, or the chain-like alkyl group as Rd¹ in Formula (d1-1), in General Formulae (y-a1-1) to (y-a1-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

In Formula (d1-1), among the examples, Rd¹ represents preferably a chain-like alkyl group which may have a substituent, more preferably a chain-like alkyl group having at least a fluorine atom as a substituent, and still more preferably a chain-like alkyl group having a fluorine atom and a hydroxy group as a substituent.

Specific preferred examples of the anion moiety in the component (d1-1) are described below.

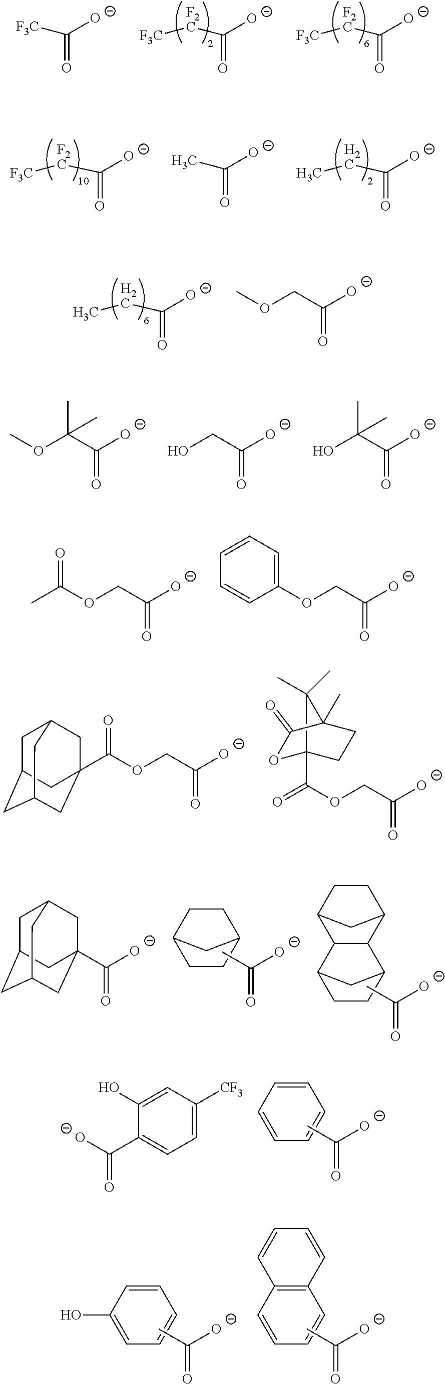

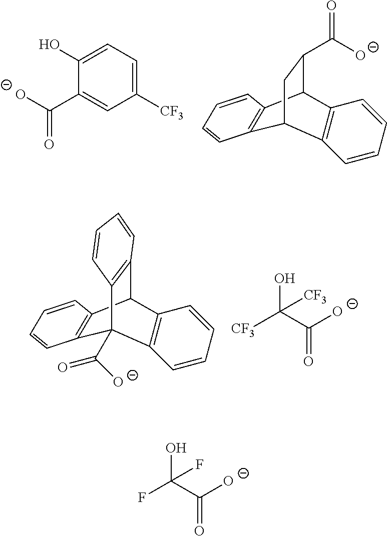 [SMILES: O=C([O-])c1cc(C(F)(F)F)ccc1O ; O=C([O-])C1CC2c3ccccc3C1c1ccccc12 ; O=C([O-])C12c3ccccc3C(c3ccccc31)c1ccccc12 ; O=C([O-])C(O)(C(F)(F)F)C(F)(F)F ; O=C([O-])C(O)(F)F]

### Cation Moiety

In Formula (di-1), M^{m+} represents an m-valent organic cation.

Suitable examples of the organic cation as M^{m+} include those for the cations each represented by General Formulae (ca-1) to (ca-5). Among these, a cation represented by General Formula (ca-1) is more preferable, and a cation represented by any of Formulae (ca-1-1) to (ca-1-66) is still more preferable.

The component (d1-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, no fluorine atom is bonded to the carbon atom adjacent to the S atom in Rd² (the carbon atom is not substituted with fluorine). In this manner, the anion of the component (d1-2) is an appropriately weak acid anion, thereby improving the quenching ability of the component (D).

Rd² represents preferably a chain-like alkyl group which may have a substituent or an aliphatic cyclic group which may have a substituent and more preferably an aliphatic cyclic group which may have a substituent.

The chain-like alkyl group preferably has 1 to 10 carbon atoms and more preferably 3 to 10 carbon atoms.

The aliphatic cyclic group is more preferably a group (which may have a substituent) obtained by removing one or more hydrogen atoms from adamantane, norbornane, isobornane, tricyclodecane, tetracyclododecane, or the like; and a group obtained by removing one or more hydrogen atoms from camphor.

The hydrocarbon group as Rd² may have a substituent, and examples of the substituent include the same groups as those for the substituent that the hydrocarbon group (such as an aromatic hydrocarbon group, an aliphatic cyclic group, or a chain-like alkyl group) as Rd¹ in Formula (d1-1) may have.

Among the examples, the anion moiety of the component (d1-2) is preferably a camphorsulfonic acid anion.

Specific preferred examples of the anion moiety in the component (d1-2) are described below.

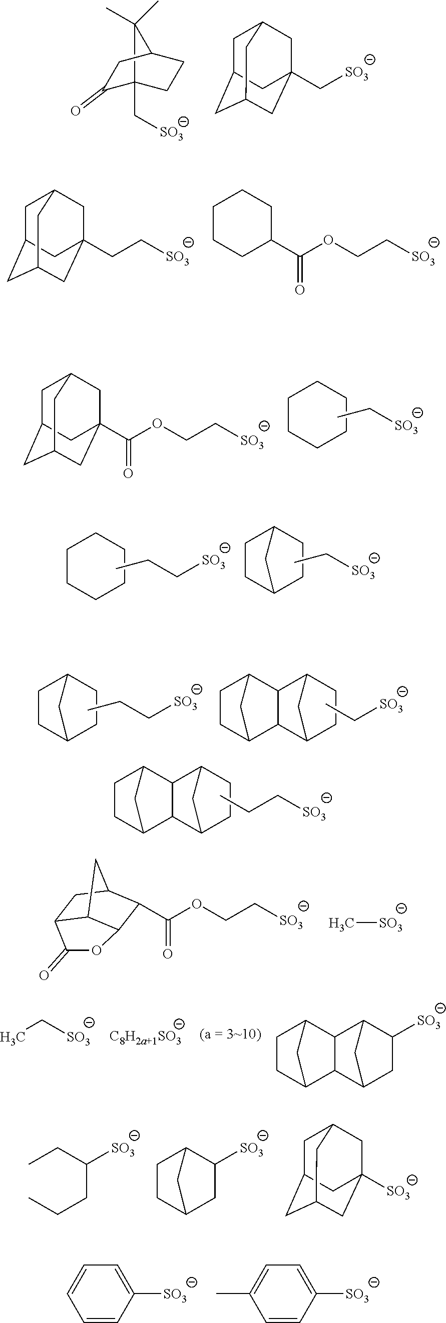

### Cation Moiety

In Formula (d1-2), M^{m+} represents an m-valent organic cation and has the same definition as that for M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹. Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a hetero atom. Examples of the divalent linking groups include the same groups as those for the divalent hydrocarbon group which may have a substituent and the divalent linking group having a hetero atom described in the section of the divalent linking group as Va^{x0} in Formula (a0-1).

It is preferable that Yd¹ represents a carbonyl group, an ester bond, an amide bond, an alkylene group, or a combination thereof. As the alkylene group, a linear or branched alkylene group is more preferable, and a methylene group or an ethylene group is still more preferable.

Specific preferred examples of the anion moiety in the component (d1-3) are described below.

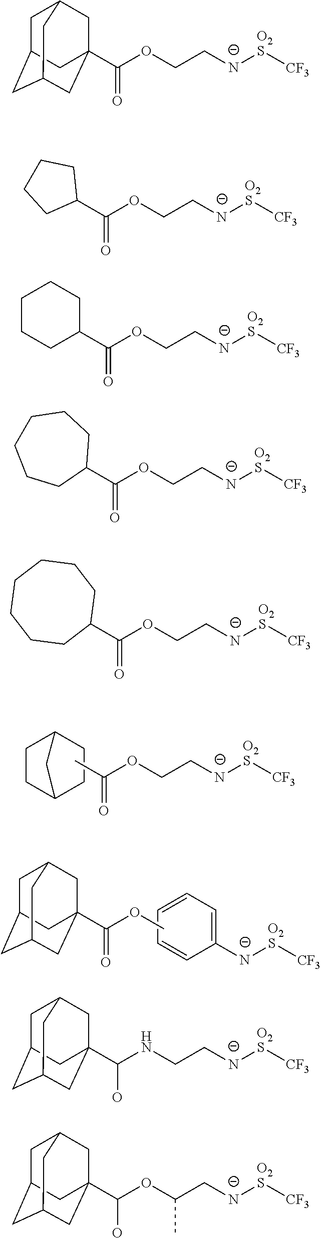

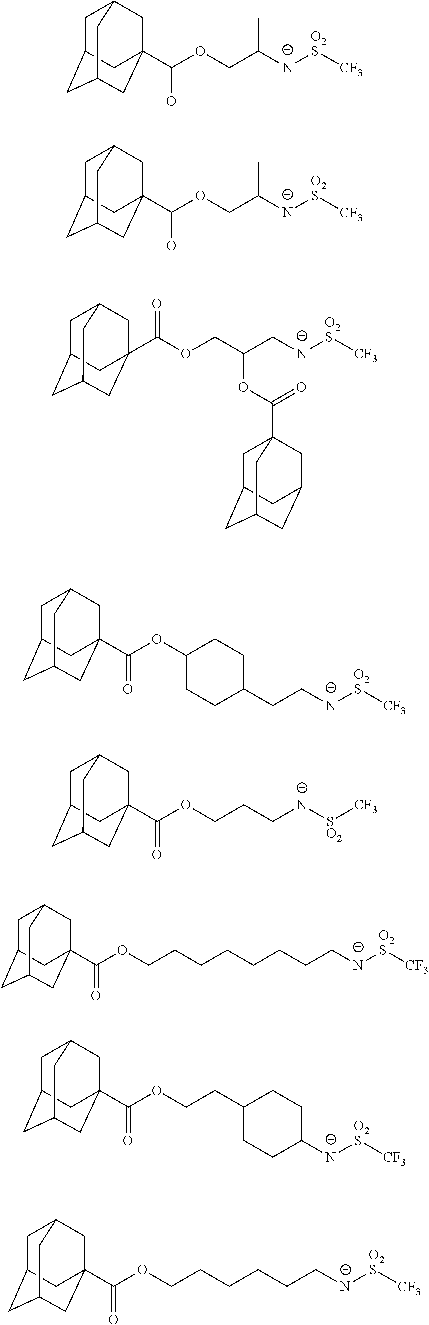

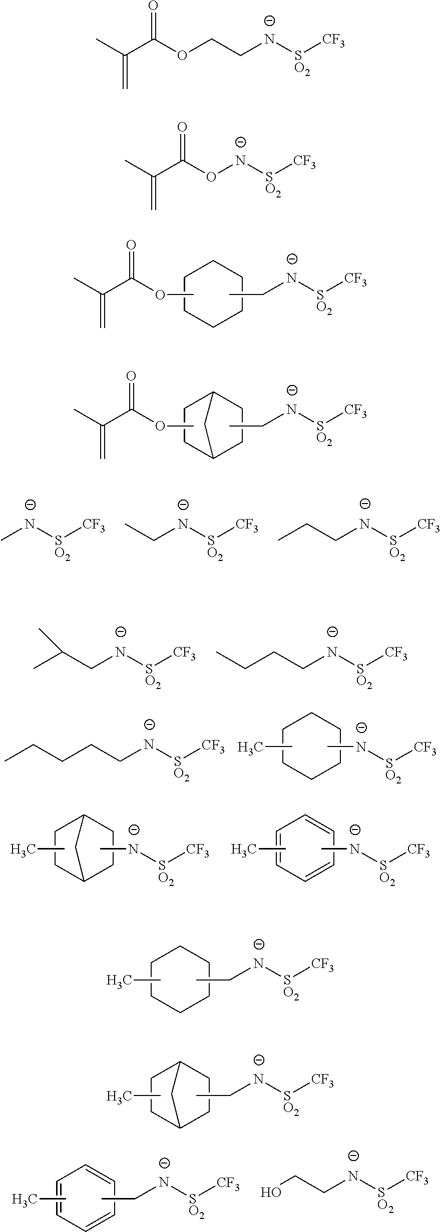

### Cation Moiety

In Formula (d1-3), M^{m+} represents an m-valent organic cation and has the same definition as that for M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 20 parts by mass, more preferably in a range of 1 to 15 parts by mass, and still more preferably in a range of 3 to 10 parts by mass with respect to 100 parts by mass of the component (A1).

In a case where the content of the component (D1) is greater than or equal to the lower limits of the above-described preferable ranges, particularly satisfactory lithography characteristics and a satisfactory resist pattern shape are likely to be obtained. In addition, in a case where the content is less than or equal to the upper limits of the above-described ranges, the sensitivity can be satisfactorily maintained and the throughput is also excellent.

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as the component functions as an acid diffusion control agent and does not correspond to the component (D1), and an optional component may be selected from known components and then used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of the aliphatic amine include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylainine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 5 to 10 carbon atoms is preferable, and tri-n-pentylamine and tri-n-octylamine are particularly preferable.

Examples of the cyclic amine include a heterocyclic compound having a nitrogen atom as a hetero atom. The heterocyclic compound may be a monocyclic compound (aliphatic monocyclic amine) or a polycyclic compound (aliphatic polycyclic amine).

Specific examples of the aliphatic monocyclic amine include piperidine and piperazine. The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

As the component (D2), an aromatic amine may be used.

Examples of the aromatic amine include 4-dimethylaminopyridine, pyrrole, indole, pyrazole, imidazole, and derivatives thereof, tribenzylamine, 2,6-diisopropylaniline, and N-tert-butoxycarbonylpyrrolidine.

Among the examples, the component (D2) is preferably an alkylamine and more preferably a trialkylamine having 5 to 10 carbon atoms.

The component (D2) may be used alone or in combination of two or more kinds thereof. In a case where the resist composition contains the component (D2), the content of the component (D2) in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (A1).

In a case where the content of the component (D2) is greater than or equal to the lower limits of the above-described preferable ranges, particularly satisfactory lithography characteristics and a satisfactory resist pattern shape are likely to be obtained. In addition, in a case where the content is less than or equal to the upper limits of the above-described ranges, the sensitivity can be satisfactorily maintained and the throughput is also excellent.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition of the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A1). In a case where the content thereof is in the above-described ranges, the lithography characteristics are further improved.

### <<Fluorine Additive Component (F)>>

The resist composition of the present embodiment may contain a fluorine additive component (hereinafter, referred to as “component (F)”) in order to impart water repellency to the resist film or to improve the lithography characteristics.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-I-adamantyl (meth)acrylate is preferable, and a constitutional unit derived from 1-ethyl-1-cyclooctyl (meth)acrylate is more preferable.

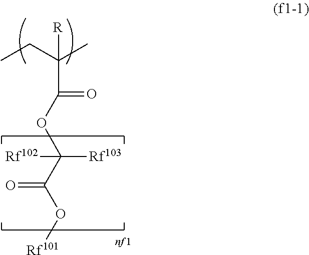

[In the formula, R has the same definition as described above, Rf¹⁰² and R¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf1 represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In General Formula (f1-1), nf1 represents an integer of 0 to 5, preferably an integer of 0 to 3, and more preferably an integer of 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or greater of the hydrogen atoms in the hydrocarbon group are fluorinated, more preferably 50% or greater thereof are fluorinated, and particularly preferably 60% or greater thereof are fluorinated from the viewpoint of increasing the hydrophobicity of the resist film during immersion exposure.

Among the examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A1).

### <<Organic Solvent Component (S)>>

The resist composition of the present embodiment can be produced by dissolving the resist materials in an organic solvent component (hereinafter, referred to as “component (S)”).

The component (S) may be any organic solvent which can dissolve each component to be used to obtain a uniform solution, and an optional organic solvent can be appropriately selected from those which have been known as solvents of a chemically amplified resist composition and then used.

Examples of the component (S) include lactones such as y-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; compounds having an ester bond, such as ethylene glycol monoacetate, diethylene glycol monoacetate, propylene glycol monoacetate, and dipropylene glycol monoacetate; polyhydric alcohol derivatives of compounds having an ether bond such as monoalkyl ether or monophenyl ether, such as monomethylether, monoethylether, monopropylether, or monobutylether of polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene, and mesitylene; and dimethylsulfoxide (DMSO).

In the resist composition of the present embodiment, the component (S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component (S). The blending ratio (mass ratio) of the mixed solvent can be appropriately determined in consideration of the compatibility between PGMEA and the polar solvent, but is preferably in the range of 1:9 to 9:1 and more preferably in a range of 2:8 to 8:2.

More specifically, in a case where EL or cyclohexanone is blended as the polar solvent, the mass ratio of PGMEA to EL or cyclohexanone is preferably in a range of 1:9 to 9:1 and more preferably in a range of 2:8 to 8:2. Further, in a case where PGME is blended as the polar solvent, the mass ratio of PGMEA to PGME is preferably in a range of 1:9 to 9:1, more preferably in a range of 2:8 to 8:2, and still more preferably in a range of 3:7 to 7:3. Further, a mixed solvent of PGMEA, PGME, and cyclohexanone is also preferable.

Further, a mixed solvent of γ-butyrolactone and at least one selected from PGMEA and EL is also preferable as the component (S). In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment, as appropriate.

After the resist material is dissolved in the component (S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition according to the present embodiment described above contains a resin component (A1) (component (A1) having the constitutional unit (a0) and the compound (D0) (component (D0)).

With the further miniaturization of a pattern, the film thickness of the resist film has been reduced. Meanwhile, the resist material is further required to have improved etching resistance and high sensitivity to radiation in a case where etching is performed by using a resist pattern as a mask.

In the resist composition according to the present embodiment described above, the sensitivity to radiation such as EUV and EB can be increased by employing the component (D0) that has an anion moiety containing a bromine atom or an iodine atom. Further, in the resist composition according to the present embodiment, the resistance to the etching process is improved by employing the component (A1) that has the constitutional unit (a0) having an aromatic carboxylic acid ester structure. Further, in the resist composition according to the present embodiment, it is considered that the insolubility in unexposed portions of the resist film is further increased and the film reduction after development is suppressed due to the synergistic effect obtained by using such a combination of the component (D0) and the component (A1).

### (Resist Pattern Formation Method)

A resist pattern formation method according to the second aspect according to the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern formation method, a resist pattern formation method by performing processes as described below is an exemplary example.

First, a support is coated with the resist composition of the present embodiment using a spinner or the like, and a bake (post applied bake (PAB)) treatment is performed under a temperature condition of 80° C. to 150° C. for 40 to 120 seconds and preferably 60 to 90 seconds to form a resist film.

Next, the resist film is exposed through a mask (mask pattern) on which a predetermined pattern is formed or directly irradiated with electron beams without using a mask pattern to be selectively exposed by drawing or the like, using an exposure device such as an electron beam lithography device or an ArF exposure device, and a baking treatment (post-exposure baking (PEB)) is carried out, for example, under a temperature condition of 80° C. to 150° C. for 40 to 120 seconds and preferably for 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

In this manner, a resist pattern can be formed.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

Further, as the support, any one of the above-described supports provided with an inorganic and/or organic film on the above-described substrate may be used. As the inorganic film, an inorganic antireflection film (inorganic BARC) can be used. As the organic film, an organic film such as an organic antireflection film (organic BARC) or a lower-layer organic film used in a multilayer resist method can be used.

Here, the multilayer resist method is a method of providing at least one layer of an organic film (lower-layer organic film) and at least one layer of a resist film (upper-layer resist film) on a substrate and performing patterning of the lower-layer organic film using a resist pattern formed on the upper-layer resist film as a mask, and this method is considered to enable formation of a pattern with a high aspect ratio. That is, according to the multilayer resist method, since a desired thickness can be ensured by the lower-layer organic film, the thickness of the resist film can be reduced, and a fine pattern with a high aspect ratio can be formed.

The multilayer resist method is basically classified into a method in which a double-layer structure consisting of an upper-layer resist film and a lower-layer organic film is formed (two-layer resist method), and a method in which a multilayer structure having three or more layers consisting of an upper-layer resist film, a lower-layer organic film, and one or more intermediate layers (thin metal film and the like) provided between the upper-layer resist film and the lower-layer organic film (three-layer resist method).

The wavelength used for light exposure is not particularly limited and the exposure can be conducted using radiation such as an ArF excimer laser, a KrF excimer laser, an F₂ excimer laser, extreme ultraviolet rays (EUV), vacuum ultraviolet rays (VUV), electron beams (EB), X-rays, and soft X-rays. The resist composition is useful for a KrF excimer laser, an ArF excimer laser, EB, and EUV, more useful for an ArF excimer laser, EB, and EUV, and particularly useful for EB and EUV. That is, the resist pattern formation method according to the present embodiment is a method particularly useful in a case where the step of exposing the resist film to light includes a process of exposing the resist film to extreme ultraviolet (EUV) rays or electron beams (EB).

The method of exposing the resist film to light may be typical exposure (dry exposure) conducted in air or an inert gas such as nitrogen, or liquid immersion exposure (liquid immersion lithography). Among these, liquid immersion exposure is preferable.

The liquid immersion exposure is an exposure method in which the region between the resist film and the lens at the lowermost position of the exposure device is filled with a solvent (liquid immersion medium) in advance that has a refractive index greater than the refractive index of air, and the exposure (immersion exposure) is conducted in this state.

As the liquid immersion medium, a solvent which has a refractive index greater than the refractive index of air but smaller than the refractive index of the resist film to be exposed to light is preferable. The refractive index of such a solvent is not particularly limited as long as the refractive index is in the above-described range. Examples of the solvent which has a refractive index greater than the refractive index of air but smaller than the refractive index of the resist film include water, a fluorine-based inert liquid, a silicon-based solvent, and a hydrocarbon-based solvent.

Specific examples of the fluorine-based inert liquid include a liquid containing a fluorine-based compound such as C₃HCl₂F₅, C₄F₉OCH₃, C₄F₉₀C₂H₅, or C₅H₃F₇ as a main component, and a liquid with a boiling point of 70° C. to 180° C. is preferable and a liquid with a boiling point of 80° C. to 160° C. is more preferable. A fluorine-based inert liquid having a boiling point in the above-described range is preferable from the viewpoint that a medium used for liquid inmiersion can be removed using a simple method after completion of light exposure.

As the fluorine-based inert liquid, a perfluoroalkyl compound in which all hydrogen atoms in the alkyl group have been substituted with fluorine atoms is particularly preferable. Specific examples of the perfluoroalkyl compound include a perfluoroalkylether compound and a perfluoroalkylamine compound.

Further, specific examples of the perfluoroalkylether compound include perfluoro(2-butyl-tetrahydrofuran) (boiling point of 102° C.), and specific examples of the perfluoroalkylamine compound include perfluorotributylamine (boiling point of 174° C.). As the liquid immersion medium, water is preferable from the viewpoints of the cost, the safety, the environmental issues, and the versatility.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

The ketone-based solvent is an organic solvent containing C—C(═O)—C in the structure thereof. The ester-based solvent is an organic solvent containing C—C(═O)—O—C in the structure thereof. The alcohol-based solvent is an organic solvent containing an alcoholic hydroxyl group in the structure thereof. The term “alcoholic hydroxyl group” indicates a hydroxyl group bonded to a carbon atom of an aliphatic hydrocarbon group. The nitrile-based solvent is an organic solvent containing a nitrile group in the structure thereof. The amide-based solvent is an organic solvent containing an amide group in the structure thereof. The ether-based solvent is an organic solvent containing C—O—C in the structure thereof.

Some organic solvents have a plurality of the functional groups which characterize each of the solvents in the structure thereof. In such a case, the organic solvents are considered to correspond to all the solvents containing the functional groups. For example, diethylene glycol monomethylether corresponds to both the alcohol-based solvent and the ether-based solvent which have been classified above.

The hydrocarbon-based solvent is a hydrocarbon solvent which is formed of a hydrocarbon that may be halogenated and does not have a substituent other than halogen atoms. Among these, a fluorine atom is preferable as the halogen atom.

Among the examples, as the organic solvent contained in the organic developing solution, a polar solvent is preferable. Further, a ketone-based solvent, an ester-based solvent, and a nitrile-based solvent are preferable.

Examples of the ketone-based solvent include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonylalcohol, acetylcarbinol, acetophenone, methyl naphthyl ketone, isophorone, propylenecarbonate, γ-butyrolactone, and methyl amyl ketone (2-heptanone). Among these examples, methyl amyl ketone (2-heptanone) is preferable as the ketone-based solvent.

Examples of the ester-based solvent include methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl-3-methoxypropionate, ethyl-3-methoxypropionate, ethyl-3-ethoxypropionate, and propyl-3-methoxypropionate. Among these examples, butyl acetate is preferable as the ester-based solvent.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used. As the surfactant, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the organic developing solution.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used. Among these, at least one solvent selected from a hydrocarbon-based solvent, a ketone-based solvent, an ester-based solvent, an alcohol-based solvent, and an amide-based solvent is preferable, at least one solvent selected from an alcohol-based solvent and an ester-based solvent is more preferable, and an alcohol-based solvent is particularly preferable.

As the alcohol-based solvent used in the rinse solution, a monohydric alcohol having 6 to 8 carbon atoms is preferable, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

These organic solvents may be used alone or in combination of two or more kinds thereof. Further, an organic solvent other than the above-described solvents and water may be mixed and used. However, in consideration of the development characteristics, the amount of water to be blended into the rinse solution is preferably 30% by mass or less, more preferably 10% by mass or less, still more preferably 5% by mass or less, and particularly preferably 3% by mass or less with respect to the total amount of the rinse solution.

A known additive can be blended into the rinse solution as necessary. Examples of the additive include a surfactant. As the surfactant, the same surfactants as those described above are exemplary examples. Among these, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the rinse solution.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

In the above-described resist pattern formation method according to the present embodiment, since the above-described resist composition is used, a resist pattern capable of further enhancing the etching resistance, achieving the high sensitivity, and suppressing the film reduction of the pattern can be formed.

### EXAMPLES

Hereinafter, the present invention will be described in more detail based on examples, but the present invention is not limited to these examples.

### <Production of Resin Component>

A polymer compound serving as a resin component for a resist composition was produced by using the following monomers.

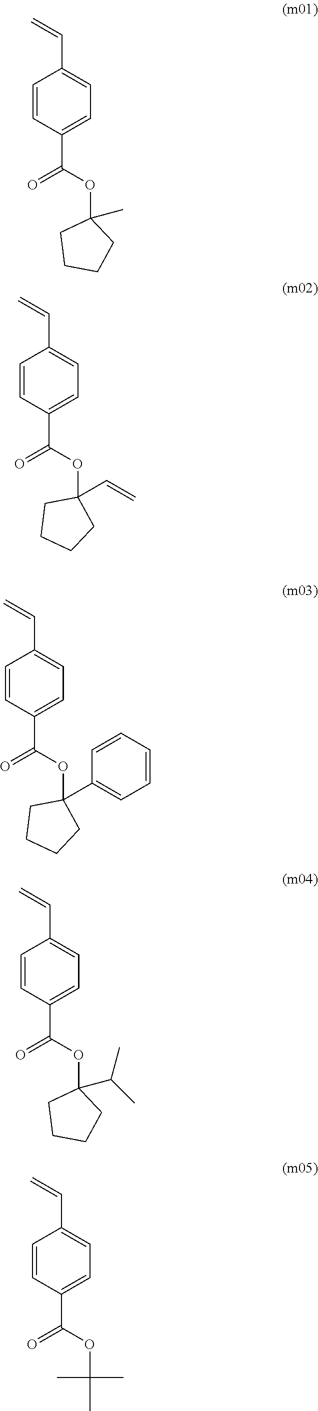 [SMILES: C=Cc1ccc(C(=O)OC2(C)CCCC2)cc1 ; C=Cc1ccc(C(=O)OC2(C=C)CCCC2)cc1 ; C=Cc1ccc(C(=O)OC2(c3ccccc3)CCCC2)cc1 ; C=Cc1ccc(C(=O)OC2(C(C)C)CCCC2)cc1 ; C=Cc1ccc(C(=O)OC(C)(C)C)cc1]

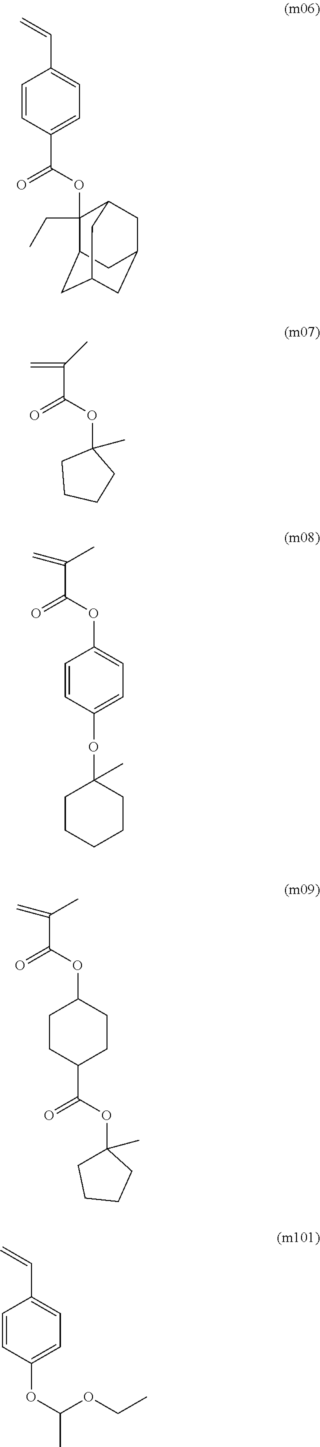

### <<Production Example 1: Production of Polymer Compound (A1-1)>>

A monomer (m01l) and a monomer (m101l) were radically polymerized at a predetermined molar ratio, and a deprotection reaction was performed, thereby obtaining a polymer compound (A1-1).

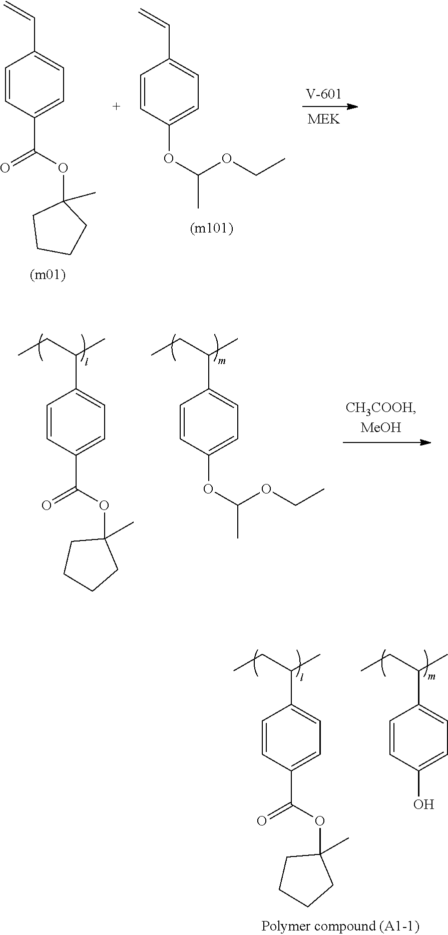

The weight-average molecular weight (Mw) and the molecular weight dispersity (Mw/Mn) of the obtained polymer compound (A1-1) were determined by GPC measurement (in terms of standard polystyrene).

The copolymerization composition ratio (the ratio (molar ratio) of each constitutional unit in the structural formula) of the obtained polymer compound (A1-1) was determined from the carbon 13 nuclear magnetic resonance spectrum (600 MHz_¹³C-NMR).

Polymer compound (A1-1): weight-average molecular weight (Mw) of 6,100, molecular weight dispersity (Mw/Mn) of 1.71, l/m=90/10 (molar ratio)

### Production Examples 2 to 4: Production of Polymer Compounds (A1-2) to (A1-4)

Each of polymer compounds (A1-2) to (A11-4) was obtained by carrying out radical polymerization and performing the deprotection reaction in the same manner as in Production Example 1 except that the blending ratio between the monomer (m01) and the monomer (m101) was changed.

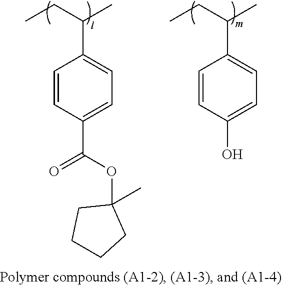

The weight-average molecular weight (Mw) and the molecular weight dispersity (Mw/Mn) of each of the obtained polymer compounds (A1-2) to (A1-4) were determined by GPC measurement (in terms of standard polystyrene). The copolymerization composition ratio (the ratio (molar ratio) of each constitutional unit in the structural formula) of each of the obtained polymer compounds (A1-2) to (A1-4) was determined from the carbon 13 nuclear magnetic resonance spectrum (600 MHz_¹³C-NMR).

Polymer compound (A1-2): weight-average molecular weight (Mw) of 6,000, molecular weight dispersity (Mw/Mn) of 1.69, l/m=80/20 (molar ratio)

Polymer compound (A1-3): weight-average molecular weight (Mw) of 6,200, molecular weight dispersity (Mw/Mn) of 1.70, l/m=60/40 (molar ratio)

Polymer compound (A1-4): weight-average molecular weight (Mw) of 6,000, molecular weight dispersity (Mw/Mn) of 1.74, l/m=40/60 (molar ratio)

### Production Examples 5 to 9: Production of Polymer Compounds (A1-5) to (A1-9)

Each of polymer compounds (A1-5) to (A1-9) was obtained by carrying out radical polymerization and performing the deprotection reaction in the same manner as in Production Example 1 except that each of the monomers (m02) to (m06) and the monomer (m101) were used at a predetermined molar ratio.

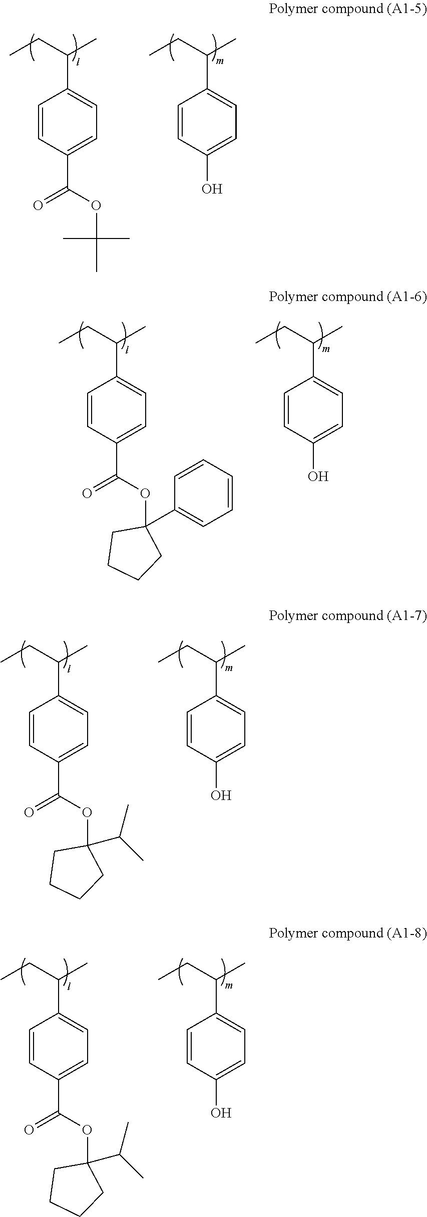

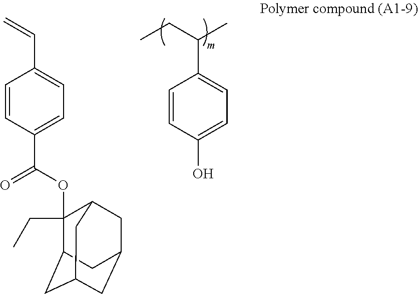

The weight-average molecular weight (Mw) and the molecular weight dispersity (Mw/Mn) of each of the obtained polymer compounds (A1-5) to (A1-9) were determined by GPC measurement (in terms of standard polystyrene). The copolymerization composition ratio (the ratio (molar ratio) of each constitutional unit in the structural formula) of each of the obtained polymer compounds (A1-5) to (A1-9) was determined from the carbon 13 nuclear magnetic resonance spectrum (600 MHz_¹³C-NMR).

Polymer compound (A1-5): weight-average molecular weight (Mw) of 6,200, molecular weight dispersity (Mw/Mn) of 1.72, l/m=60/40 (molar ratio) Polymer compound (A1-6): weight-average molecular weight (Mw) of 6,300, molecular weight dispersity (Mw/Mn) of 1.69, l/m=60/40 (molar ratio) Polymer compound (A1-7): weight-average molecular weight (Mw) of 6,100, molecular weight dispersity (Mw/Mn) of 1.70, l/m=60/40 (molar ratio) Polymer compound (A1-8): weight-average molecular weight (Mw) of 6,000, molecular weight dispersity (Mw/Mn) of 1.71, l/m=60/40 (molar ratio) Polymer compound (A1-9): weight-average molecular weight (Mw) of 6,100, molecular weight dispersity (Mw/Mn) of 1.73, l/m=60/40 (molar ratio) Production Examples 10 to 12: Production of Polymer Compounds (A2-1) to (A2-3)

Each of polymer compounds (A2-1) to (A2-3) was obtained by carrying out radical polymerization and performing the deprotection reaction in the same manner as in Production Example 1 except that each of the monomers (m07) to (m09) and the monomer (m101) were used at a predetermined molar ratio.

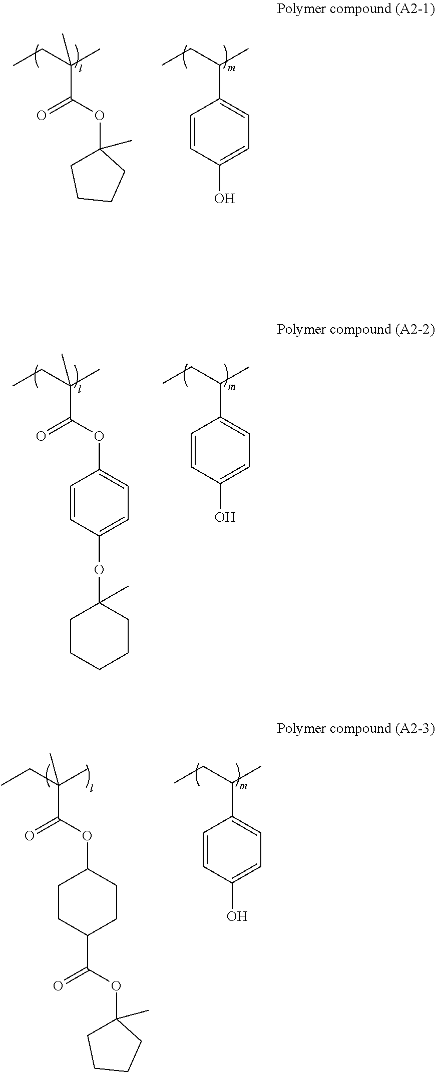

The weight-average molecular weight (Mw) and the molecular weight dispersity (Mw/Mn) of each of the obtained polymer compounds (A2-1) to (A2-3) were determined by GPC measurement (in terms of standard polystyrene). The copolymerization composition ratio (the ratio (molar ratio) of each constitutional unit in the structural formula) of each of the obtained polymer compounds (A2-1) to (A2-3) was determined from the carbon 13 nuclear magnetic resonance spectrum (600 MHz_¹³C-NMR).

Polymer compound (A2-1): weight-average molecular weight (Mw) of 6,200, molecular weight dispersity (Mw/Mn) of 1.73, l/m=60/40 (molar ratio)

Polymer compound (A2-2): weight-average molecular weight (Mw) of 6,200, molecular weight dispersity (Mw/Mn) of 1.72, l/m=60/40 (molar ratio)

Polymer compound (A2-3): weight-average molecular weight (Mw) of 6,300, molecular weight dispersity (Mw/Mn) of 1.72, l/m=60/40 (molar ratio)

### <Preparation of Resist Composition>

### Examples 1 to 19 and Comparative Examples 1 to 4

Each of the components shown in Tables 1 and 2 was mixed and dissolved to prepare a resist composition of each Example.

TABLE 1

| | Component (A) | | | Component (D) | | |
|---|---|---|---|---|---|---|
| | Component | Component | Component | Component | Component | Component |
| | (A1) | (A2) | (B) | (D0) | (D1) | (S) |
| Example | (A)-1 | — | (B)-1 | (D)-1 | — | (S)-1 |
| 1 | [100] | | [16.9] | [6.9] | | [8000] |
| Example | (A)-2 | — | (B)-1 | (D)-1 | — | (S)-1 |
| 2 | [100] | | [16.9] | [6.9] | | [8000] |
| Example | (A)-3 | — | (B)-1 | (D)-1 | — | (S)-1 |
| 3 | [100] | | [16.9] | [6.9] | | [8000] |
| Example | (A)-4 | — | (B)-1 | (D)-1 | — | (S)-1 |
| 4 | [100] | | [16.9] | [6.9] | | [8000] |
| Example | (A)-3 | — | (B)-1 | (D)-2 | — | (S)-1 |
| 5 | [100] | | [16.9] | [5.6] | | [8000] |
| Example | (A)-3 | — | (B)-1 | (D)-3 | — | (S)-1 |
| 6 | [100] | | [16.9] | [5.6] | | [8000] |
| Example | (A)-3 | — | (B)-1 | (D)-4 | — | (S)-1 |
| 7 | [100] | | [16.9] | [7.2] | | [8000] |
| Example | (A)-3 | — | (B)-1 | (D)-5 | — | (S)-1 |
| 8 | [100] | | [16.9] | [8.5] | | [8000] |
| Example | (A)-5 | — | (B)-1 | (D)-1 | — | (S)-1 |
| 9 | [100] | | [16.9] | [6.9] | | [8000] |
| Example | (A)-6 | — | (B)-1 | (D)-1 | — | (S)-1 |
| 10 | [100] | | [16.9] | [6.9] | | [8000] |
| Example | (A)-7 | — | (B)-1 | (D)-1 | — | (S)-1 |
| 11 | [100] | | [16.9] | [6.9] | | [8000] |
| Example | (A)-8 | — | (B)-1 | (D)-1 | — | (S)-1 |
| 12 | [100] | | [16.9] | [6.9] | | [8000] |
| Example | (A)-9 | — | (B)-1 | (D)-1 | — | (S)-1 |
| 13 | [100] | | [16.9] | [6.9] | | [8000] |
| Example | (A)-3 | — | (B)-1 | (D)-1 | — | (S)-1 |
| 14 | [100] | | [16.9] | [2.1] | | [8000] |
| Example | (A)-3 | — | (B)-1 | (D)-1 | — | (S)-1 |
| 15 | [100] | | [16.9] | [4.1] | | [8000] |
| Example | (A)-3 | — | (B)-1 | (D)-1 | — | (S)-1 |
| 16 | [100] | | [16.9] | [8.9] | | [8000] |
| Example | (A)-3 | — | (B)-1 | (D)-1 | — | (S)-1 |
| 17 | [100] | | [16.9] | [11.0] | | [8000] |
| Example | (A)-3 | — | (B)-2 | (D)-1 | — | (S)-1 |
| 18 | [100] | | [25.7] | [6.9] | | [8000] |
| Example | (A)-3 | — | (B)-3 | (D)-1 | — | (S)-1 |
| 19 | [100] | | [19.0] | [6.9] | | [8000] |

TABLE 2

| | Component (A) | | | Component (D) | | |
|---|---|---|---|---|---|---|
| | Component | Component | Component | Component | Component | Component |
| | (A1) | (A2) | (B) | (D0) | (D1) | (S) |
| Comparative | — | (A)-10 | (B)-1 | (D)-1 | — | (S)-1 |
| Example | | [100] | [16.9] | [6.9] | | [8000] |
| 1 | | | | | | |
| Comparative | (A)-3 | — | (B)-1 | — | (D)-6 | (S)-1 |
| Example | [100] | | [16.9] | | [4.2] | [8000] |
| 2 | | | | | | |
| Comparative | — | (A)-11 | (B)-1 | — | (D)-6 | (S)-1 |
| Example | | [100] | [16.9] | | [4.2] | [8000] |
| 3 | | | | | | |
| Comparative | — | (A)-12 | (B)-1 | — | (D)-6 | (S)-1 |
| Example | | [100] | [16.9] | | [4.2] | [8000] |
| 4 | | | | | | |

In Tables 1 and 2, each abbreviation has the following meaning. The numerical values in the brackets are blending amounts (parts by mass).
(A)-1 to (A)-9: polymer compounds (A1-1) to (A1-9) shown above
(A)-10 to (A)-12: polymer compounds (A2-1) to (A2-3) shown above
(B)-1: acid generator composed of compound represented by Chemical Formula (B-1)
(B)-2: acid generator consisting of compound represented by Chemical Formula (B-2)
(B)-3: acid generator consisting of compound represented by Chemical Formula (B-3)

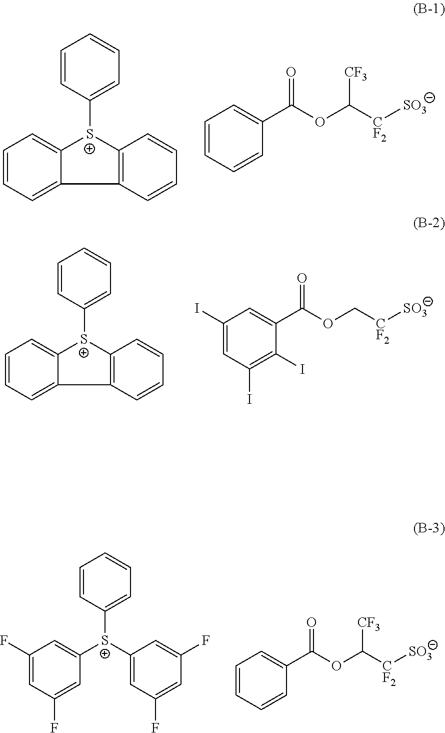

(D)-1 to (D)-5: acid diffusion control agents formed of compounds each represented by Chemical Formulae (DO-1) to (DO-5)

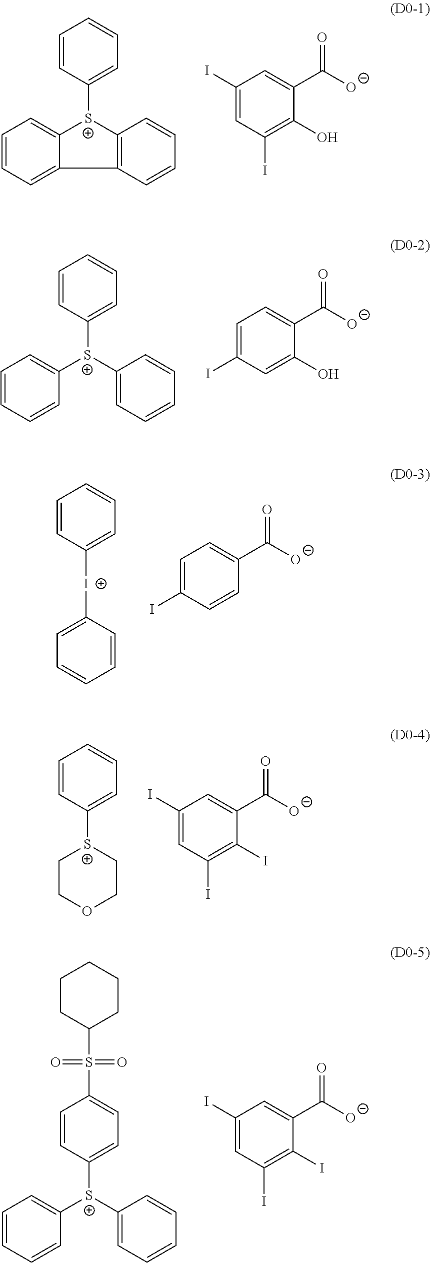

(D)-6: acid diffusion control agent formed of compound represented by Chemical Formula (D1-1)

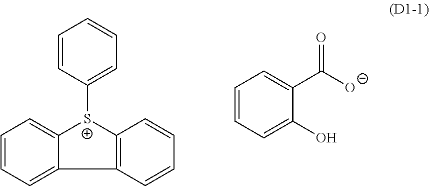 [SMILES: c1ccc(-[s+]2c3ccccc3c3ccccc32)cc1 ; O=C([O-])c1ccccc1O]

(S)-1: mixed solvent of propylene glycol monomethyl ether acetate/propylene glycol monomethyl ether=60/40 (mass ratio)

### <Resist Pattern Formation>

The resist composition of each Example was applied onto an 8-inch silicon substrate which had been subjected to a hexamethyldisilazane (HMDS) treatment using a spinner, the coated wafer was subjected to a pre-baking (PAB) treatment on a hot plate at a temperature of 110° C. for 60 seconds so that the coated wafer was dried to form a resist film having a film thickness of 50 nm.

Next, drawing (exposure) was carried out on the resist film by using an electron beam lithography device JEOL-JBX-9300FS (manufactured by JEOL Ltd.), with the target size set to a 1:1 line and space pattern (hereinafter, referred to as “LS pattern”) of a line width of 50 nm, at an acceleration voltage of 100 kV. Thereafter, a post exposure bake (PEB) treatment was performed thereon at 100° C. for 60 seconds.

Subsequently, alkali development was performed at 23° C. for 60 seconds using a 2.38 mass % tetramethylammonium hydroxide (TMAH) aqueous solution “NMD-3” (trade name, manufactured by TOKYO OHKA KOGYO CO., LTD.).

Thereafter, water rinsing was performed for 15 seconds using pure water.

As a result, a 1:1 LS pattern having a line width of 50 nm was formed.

### [Evaluation of Optimum Exposure Amount (Eop)]

The optimum exposure amount Eop (μC/cm²) at which the LS pattern having the target size was formed as determined according to <resist pattern formation> described above. The results are listed in the columns of “Eop (μC/cm²)” in Table 3.

### [Evaluation of Etching Resistance]

The resist composition of each Example was applied onto an 8-inch silicon substrate which had been subjected to a hexamethyldisilazane (HMDS) treatment using a spinner, the coated wafer was subjected to a pre-baking (PAB) treatment on a hot plate at a temperature of 110° C. for 60 seconds so that the coated wafer was dried to form a resist film having a film thickness of 50 nm.

Next, each resist film was subjected to a dry etching treatment with CF₄ gas for 60 seconds using a dry etching device TCA-3822 (trade name, manufactured by TOKYO OHKA KOGYO CO., LTD.). The residual film amount was determined from the film thicknesses of the resist film before and after the dry etching treatment.

Further, the residual film amount of the resist film formed by using the resist composition of each example was listed in Table 3 as the relative value in a case where the residual film amount of the resist film formed by using the resist composition of Comparative Example 1 was set to 1.00. The results are listed in the columns of “etching resistance” in Table 3.

### [Evaluation of Film Reduction]

The film thickness after the PAB treatment (the film thickness of the unexposed portions with a large area) and the film thickness after the film was rinsed with water were each measured according to <resist pattern formation> described above to determine the film reduction amount. The results are listed in the columns of “film reduction rate (%)” in Table 3.

TABLE 3

| | | | | | Film |
|---|---|---|---|---|---|
| | PAB | PEB | Eop | Etching | reduction |
| | (° C.) | (° C.) | (μC/cm²) | resistance | rate (%) |
| Example 1 | 110 | 100 | 85 | 1.11 | 99.1 |
| Example 2 | 110 | 100 | 84 | 1.12 | 98.9 |
| Example 3 | 110 | 100 | 83 | 1.14 | 98.6 |
| Example 4 | 110 | 100 | 82 | 1.14 | 98.3 |
| Example 5 | 110 | 100 | 83 | 1.12 | 98.4 |
| Example 6 | 110 | 100 | 85 | 1.13 | 98.4 |
| Example 7 | 110 | 100 | 84 | 1.13 | 98.7 |
| Example 8 | 110 | 100 | 83 | 1.14 | 98.6 |
| Example 9 | 110 | 100 | 82 | 1.16 | 98.2 |
| Example 10 | 110 | 100 | 82 | 1.16 | 98.5 |
| Example 11 | 110 | 100 | 85 | 1.14 | 98.3 |
| Example 12 | 110 | 100 | 84 | 1.12 | 98.7 |
| Example 13 | 110 | 100 | 83 | 1.15 | 98.9 |
| Example 14 | 110 | 100 | 82 | 1.13 | 97.9 |
| Example 15 | 110 | 100 | 84 | 1.13 | 98.2 |
| Example 16 | 110 | 100 | 85 | 1.15 | 98.5 |
| Example 17 | 110 | 100 | 87 | 1.15 | 99.0 |
| Example 18 | 110 | 100 | 80 | 1.15 | 99.0 |
| Example 19 | 110 | 100 | 79 | 1.16 | 98.9 |
| Comparative | 110 | 100 | 99 | 1.00 | 90.2 |
| Example 1 | | | | | |
| Comparative | 110 | 100 | 106 | 1.05 | 93.8 |
| Example 2 | | | | | |
| Comparative | 110 | 100 | No Pattern | | |
| Example 3 | | | | | |
| Comparative | 110 | 100 | 106 | 0.92 | 96.8 |
| Example 4 | | | | | |

### Example 3

As listed in Table 3, it was confirmed that a resist pattern in which the etching resistance was further enhanced, high sensitivity was achieved, and film reduction of a pattern was suppressed was formed with the resist compositions of Examples 1 to 19 as compared with the resist compositions of Comparative Examples 1, 2, and 4.

In a case of the resist composition of Comparative Example 3, the entire resist film was dissolved in an alkali developing solution, and thus a resist pattern could not be formed (described as “no pattern” in Table 3).

While preferred embodiments of the present invention have been described and illustrated above, it should be understood that these are exemplary of the present invention and are not to be considered as limiting. Additions, omissions, substitutions, and other modifications can be made without departing from the scope of the invention. Accordingly, the present invention is not to be considered as being limited by the foregoing description and is only limited by the appended claims.

What is claimed is:

1. A resist composition which generates an acid upon light exposure and whose solubility in a developing solution is changed due to an action of an acid, the resist composition comprising:
a resin component (A1) whose solubility in a developing solution is changed due to an action of an acid; and
a compound (D0) represented by General Formula (d0-1),
wherein the resin component (A1) has a constitutional unit (a0) represented by General Formula (a0-1), 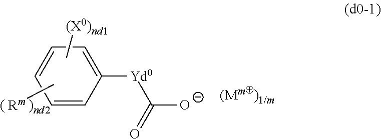
wherein X⁰ represents a bromine atom or an iodine atom, R^{m} represents a hydroxy group, an alkyl group, a fluorine atom, or a chlorine atom, nd1 represents an integer of 1 to 5, nd2 represents an integer of 0 to 4, where 1≤nd1+nd2≤5 is satisfied, Yd⁰ represents a divalent linking group or a single bond, M^{m+} represents an m-valent organic cation, and m represents an integer of I or greater} 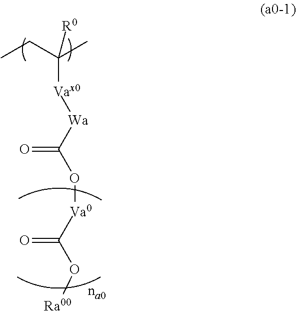
wherein R⁰ represents a hydrogen atom, an alkyl group having 1 to 5 carbon atoms, a halogen atom, or a halogenated alkyl group having 1 to 5 carbon atoms, Va^{x0} represents a single bond or a divalent linking group, Wa represents a divalent aromatic hydrocarbon group which may have a substituent, Va⁰ represents a divalent hydrocarbon group which may have an ether bond, nₐ₀ represents an integer of 0 to 2, and Ra⁰⁰ represents an acid dissociable group.

2. The resist composition according to claim 1, further comprising an acid generator component (B) which generates an acid upon light exposure.

3. The resist composition according to claim 1, wherein the resin component (A1) further comprises a constitutional unit (a10) represented by General Formula (a10-1): 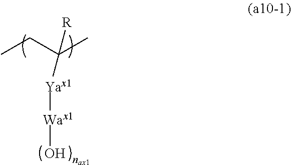
wherein R represents a hydrogen atom, an alkyl group having 1 to 5 carbon atoms, or a halogenated alkyl group having 1 to 5 carbon atoms, Ya^{x1} represents a single bond or a divalent linking group, Wa^{x1} represents an (nₐₓ₁+1)-valent aromatic hydrocarbon group, and nₐₓ₁ represents an integer of 1 or greater.

4. A resist pattern formation method comprising:
forming a resist film on a support using the resist composition according to claim 1;
exposing the resist film to light; and
developing the resist film exposed to light to form a resist pattern.
